(19) United States
(12) Patent Application Publication
(10) Pub. No.: US 2023/0000870 A1
(43) Pub. Date: Jan. 5, 2023

(54) METHODS OF TREATING CANCER IN BIOMARKER-IDENTIFIED PATIENTS WITH INHIBITORS OF CYCLIN-DEPENDENT KINASE 7 (CDK7)

(71) Applicant: Syros Pharmaceuticals, Inc., Cambridge, MA (US)

(72) Inventors: John Graeme Hodgson, Boxborough, MA (US); Liv Helena Johannessen, Boston, MA (US)

(21) Appl. No.: 17/772,747

(22) PCT Filed: Oct. 29, 2020
(86) PCT No.: PCT/US2020/057998

§ 371 Date: Apr. 28, 2022

Related U.S. Application Data
(60) Provisional application No. 62/927,561, filed on Oct. 29, 2019.

(51) Int. Cl. A61K 31/519; A61K 31/4162; A61K 45/06

(52) U.S. Cl. CPC A61K 31/519; A61K 31/4162; A61K 45/06

(57) ABSTRACT

The present invention relates to methods of identifying patients suffering from various types of cancer who are more likely to respond to treatment with CDK7 inhibitor described herein, or a pharmaceutically acceptable salt thereof, either when administered or used alone or in combination with a second therapeutic agent (e.g., another anti-cancer therapy). Patients are identified based on one or more features (e.g., gene copy number or expression level) of certain biomarkers (e.g., RB1 or another member of the E2F pathway). In addition, the present invention relates to methods of treating an identified patient with a CDK7 inhibitor described herein, or a pharmaceutically acceptable salt thereof, either alone or in combination with a second therapeutic agent. In another aspect, the present invention features kits including instructions for treating a patient identified as described herein.

### CROSS-REFERENCE TO RELATED APPLICATIONS

This application claims the benefit of the filing date of U.S. provisional application No. 62/927,561, filed Oct. 29, 2019, the content of which is hereby incorporated herein by reference in its entirety.

### BACKGROUND OF THE INVENTION

The long evolution of healthcare has reached a point in time where the promise of biomarker analysis is beginning to be realized. When physicians can stratify patients, even those who share many similar physiological traits and exhibit common symptoms of a given disease, into more specific groups, they can better tailor treatment and optimize the outcome for each patient. However, it is challenging to develop molecular diagnostics, and few are commercially available.

### SUMMARY OF THE INVENTION

The present invention features, inter alia, diagnostic methods for identifying cancer patients for treatment with a CDK7 inhibitor described herein (i.e., diagnostic methods for selecting a patient for treatment) and methods for treating identified patients with such an inhibitor, either alone or in combination with one or more additional therapeutic agents (e.g., a second anti-cancer agent), as described further below. The diagnostic methods include a step of identifying a patient suffering from a cancer that is likely to respond well to treatment with a CDK7 inhibitor disclosed herein, as shown and described further below. The treatment methods include a step of administering such a CDK7 inhibitor to an identified patient, whose response can be, for example, significant tumor growth inhibition (TGI; e.g., more than about 80-90% TGI and/or continued tumor suppression even after cessation of treatment). Thus, the present invention encompasses methods in which a patient is only diagnosed as being a good candidate for treatment (i.e., identified for treatment), methods in which a patient who has been determined to be a good candidate for treatment (e.g., previously identified) is treated, and methods requiring that a patient be both diagnosed and treated as described herein.

When we refer to “a CDK7 inhibitor described herein” we mean compounds including any one of those described herein, including compounds of Formula (I):

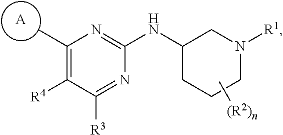

or a pharmaceutically acceptable salt thereof, wherein

ring A is a bicyclic 6,5-ring system selected from:

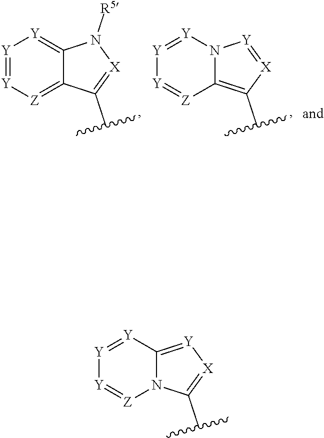

and comprises no more than four ring nitrogen atoms;

X is N or C(R⁶), wherein R⁶ is hydrogen, —CN, —CH₃, —CH₂F, —CHF₂ or —CF₃;

each Y is, independently, N or C(R⁷), wherein R⁷ is hydrogen or R⁵;

Z is N or C(R⁸), wherein R⁸ is hydrogen or fluoro;

R¹ is hydrogen, —C₁-C₆ alkyl, —O—(C₁-C₆-alkylene)-O—(C₁-C₄-alkyl), —(C₀-C₆ alkylene)-(C₃-C₈ cycloalkyl), —(C₁-C₆ alkylene)-heterocyclyl, —(C₁-C₆ alkylene)-heteroaryl, —(C₁-C₆ alkylene)-N(R^{1′})₂, —(C₁-C₆ alkylene)-NR^{1′}—S(O)₂—(C₁-C₄ alkyl), —(C₁-C₆ alkylene)-NR^{1′}—SO₂—N(R^{1′})₂, —(C₁-C₆ alkylene)-S(O)₂—(C₁-C₄ alkyl), or —(C₁-C₆ alkylene-S(O)₂—N(R^{1′})₂, wherein any alkyl, alkylene, cycloalkyl, heterocyclyl or heteroaryl portion of R¹ is optionally substituted:

each R^{1′} is, independently, hydrogen or optionally substituted C₁-C₆ alkyl, or

two R^{1′} are optionally taken together with the nitrogen atom to which they are bound to form a 4-6 membered, optionally substituted heterocyclyl or heteroaryl ring comprising up to 2 additional heteroatoms selected from N, O, and S, wherein:

each R², if present, is, independently, halo, —OH, —CN, —C₁-C₆ alkyl, —(C₀-C₆ alkylene)-(C₃-C₈ cycloalkyl), —(C₀-C₆ alkylene)-heterocyclyl, —(C₀-C₆ alkylene)-heteroaryl, —(C₀-C₆ alkylene)-aryl, —(C₀-C₆ alkylene)-C(O)-heterocyclyl, —(C₀-C₆ alkylene)-C(O)-heteroaryl, —O—(C₁-C₆-alkyl); —O—(C₁-C₆-alkylene)-O—(C₁-C₄-alkyl); —O—(C₁-C₄-alkylene)-(C₃-C₈ cycloalkyl), —O—(C₁-C₆-alkylene)-heterocyclyl, —O—(C₁-C₆-alkylene)-heteroaryl, or —O—(C₁-C₆-alkylene)-aryl, or

R¹ and any R² are taken together with the atoms to which they are bound to form an optionally substituted heterocyclyl or heteroaryl ring fused, spirofused or bridged to the piperidine ring, or

two R² are taken together to form oxo (═O), or taken together with the atom or atoms to which they are bound and any intervening ring atoms to form an optionally substituted aryl, cycloalkyl, heterocyclyl or heteroaryl ring fused, spirofused or bridged to the piperidine ring,

wherein any alkyl, alkylene, cycloalkyl, heterocyclyl or heteroaryl portion of R², any ring formed by taking R¹ together with R², or any ring formed by taking two R² together is optionally substituted:

R³ is hydrogen, halo, —CN, optionally substituted —C₁-C₆ alkyl, or optionally substituted C₃-C₈ cycloalkyl;

R⁴ is halo, —CN, —C₁-C₆ alkyl, —C₂-C₆ alkenyl, C₂-C₆ alkynyl, —O—C₁-C₆ alkyl, —S—C₁-C₆ alkyl, or a C₃-C₈ cycloalkyl, wherein any alkyl, alkenyl, or alkynyl portion of R⁴ is optionally substituted;

each R⁵ is, independently, halo, —OH, —C₁-C₆ alkyl, —CN, —(C₀-C₆ alkylene)-C(O)OH, —(C₀-C₆ alkylene)-C(O)—(C₁-C₄ alkyl), —(C₀-C₆ alkylene)-C(O)—N(R^{1′})₂, —(C₀-C₆ alkylene)-S(O)₂—(C₁-C₄ alkyl), —(C₀-C₆ alkylene)-S(O)₂—N(R^{1′})₂, —(C₀-C₆ alkylene)-P(O)—O—(C₁-C₄ alkyl)₂, —(C₀-C₆ alkylene)-P(O)—(C₁-C₄ alkyl)(O—C₁-C₄ alkyl), —(C₀-C₆ alkylene)-P(O)(C₁-C₄ alkyl)₂, —(C₀-C₆ alkylene)-(C₃-C₈ cycloalkyl), —(C₀-C₆ alkylene)-heterocyclyl, —(C₀-C₆ alkylene)-heteroaryl, —(C₀-C₆ alkylene)-C(O)-heterocyclyl, —(C₀-C₆ alkylene)-C(O)-heteroaryl, —O—(C₁-C₆-alkyl), —O—(C₁-C₆-alkylene)-O—(C₁-C₄-alkyl), —O—(C₀-C₆-alkylene)-(C₃-C₈ cycloalkyl), —O—(C₁-C₆-alkylene)-heterocyclyl, or —O—(C₁-C₆-alkylene)-heteroaryl, wherein any alkyl, alkylene, cycloalkyl, heterocyclyl and heteroaryl portion of R⁵ is optionally substituted; or

two vicinal R⁵ are taken together with the ring atoms to which they are bound to form an optionally substituted cycloalkyl or optionally substituted heterocyclyl, wherein each cycloalkyl or heterocyclyl is fused to ring A;

R^{5′} is hydrogen, —CN, —C₁-C₆ alkyl, —(C₀-C₆ alkylene)-S(O)₂—N(R^{1′})₂, —(C₀-C₆ alkylene)-(C₃-C₈ cycloalkyl), —(C₀-C₆ alkylene)-C(O)—N(R^{1′})₂, —(C₀-C₆ alkylene)-aryl, —(C₀-C₆ alkylene)-heterocyclyl, —(C₀-C₆ alkylene)-heteroaryl, —(C₀-C₆ alkylene)-S(O)₂—(C₁-C₄ alkyl), —(C₁-C₆ alkylene)-O—(C₁-C₃ alkylene)-C(O)—N(R^{1′})₂, —(C₁-C₆ alkylene)-O—(C₁-C₄ alkylene)-P(O)(C₁-C₄ alkyl)₂, —(C₁-C₆ alkylene)-O—(C₁-C₄ alkylene)-P(O)(C₁-C₄ alkyl)-O—(C₁-C₄ alkyl), —(C₁-C₆ alkylene)-O—(C₁-C₄ alkylene)-P(O)—(O—C₁-C₄ alkyl)₂, —(C₁-C₆ alkylene)-O—(C₁-C₄ alkylene)-S(O)₂—(C₁-C₄ alkyl), —(C₁-C₆ alkylene)-O—(C₁-C₄ alkylene)-S(O)₂—N(R^{1′})₂, —(C₁-C₆ alkylene)-O—(C₁-C₄ alkyl), —(C₁-C₆ alkylene)-O—(C₃-C₈ cycloalkyl), —(C₁-C₆ alkylene)-O-heteroaryl, —(C₁-C₆ alkylene)-O-heterocyclyl, —(C₁-C₆ alkylene)-P(O)(C₁-C₄ alkyl)₂, —(C₁-C₆ alkylene)-P(O)(C₁-C₄ alkyl)-O—(C₁-C₄ alkyl), —(C₁-C₆ alkylene)-P(O)—(O—C₁-C₄ alkyl)₂, —(C₁-C₆ alkylene)-C(O)—(C₁-C₄ alkyl), or —(C₁-C₆ alkylene)-C(O)OH, wherein any alkyl, alkylene, cycloalkyl, heterocyclyl or heteroaryl portion of R^{5′} is optionally substituted; and

n is 0, 1, 2, 3, or 4. In case of doubt, each such compound is “a CDK7 inhibitor described herein,” as are each of the embodiments and distinct compounds described below.

In various embodiments, R¹ is —C(O)—O—(C₁-C₆ alkyl) or —(C₀-C₆ alkylene)-carbocyclyl, wherein carbocyclyl is optionally substituted.

In various embodiments, each R², if present, is —NH—C(O)—C₁-C₄ alkyl, —C(O)—NH— (unsubstituted C₁-C₄ alkyl), —(C₀-C₆ alkylene)-carbocyclyl or —O—(C₁-C₄-alkylene)-carbocyclyl, wherein each alkylene or carbocyclyl is optionally substituted.

In some embodiments, R³ is additionally selected from optionally substituted carbocyclyl.

In some embodiments, R⁴ is additionally selected from optionally substituted carbocyclyl.

In some embodiments, each R⁵ is additionally selected from —(C₀-C₆ alkylene)-carbocyclyl, —O—(C₀-C₆-alkylene)-carbocyclyl, phenyl, —(C₂-C₄ alkenylene)-phenyl, —S(O)—(C₁-C₄ alkyl), —S—(C₁-C₄ alkyl), —S(O)—OH, and —S(O)₂—OH, wherein any alkyl, alkylene, alkenylene, carbocyclyl, or phenyl is optionally substituted.

In some embodiments, R^{5′} and any R⁵ are taken together with the ring atoms to which they are bound to form an optionally substituted heterocyclyl, wherein each heterocyclyl is fused to ring A.

In some embodiments, the compound of Formula (I) is not:

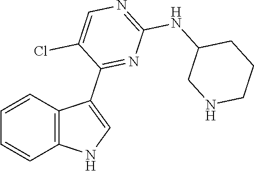 [SMILES: Clc1cnc(NC2CCCNC2)nc1-c1c[nH]c2ccccc12]

or a pharmaceutical salt of the foregoing.

In some embodiments, the compound of Formula (I) is not:

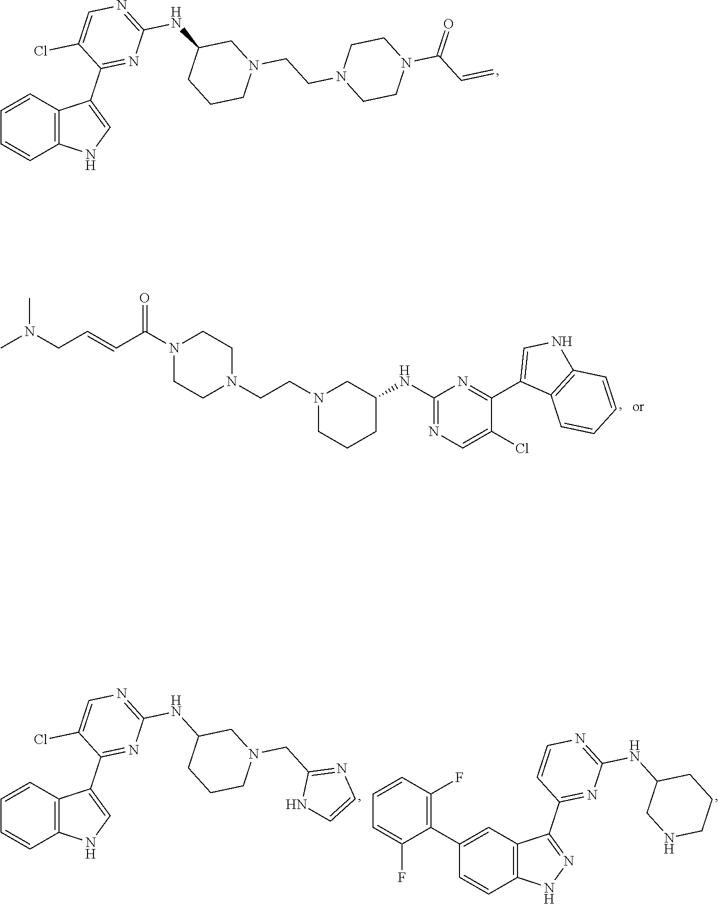

or a pharmaceutically acceptable salt of any of the foregoing.

In some embodiments, ring A is:

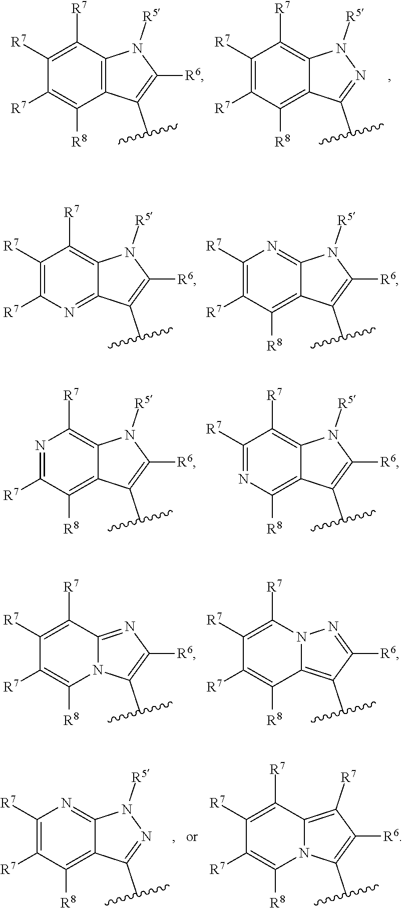

In some embodiments, ring A is indol-3-yl or indazol-3-yl. In some embodiments, ring A is indol-3-yl. In some embodiments, ring A is:

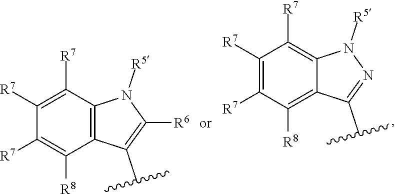

In some embodiments, any alkyl or alkylene portion of R¹ is optionally substituted with one or more independently selected monovalent substituents (e.g., such substituents do not include ═O).

In some embodiments, any heterocyclyl or heteroaryl portion of R¹ is optionally and independently substituted with one or more substituents independently selected from halo, C₁-C₄ alkyl, C₃-C₆ cycloalkyl, —OH, ═O, —CN, —C(O)N(R^{1′})₂, —S(O)₂—(C₁-C₄-alkyl), and —S(O)₂—N(R^{1′})₂; and any alkyl, alkylene, or cycloalkyl portion of R¹ or a substituent thereon is optionally substituted with one or more substituents independently selected from fluorine, OH and CN.

In some embodiments, R¹ is hydrogen, —C₁-C₆ alkyl, —O—(C₁-C₆-alkylene)-O—(C₁-C₄-alkyl), —(C₁-C₆ alkylene)-N(R^{1′})₂, —(C₁-C₆ alkylene)-NR^{1′}—S(O)₂—(C₁-C₄ alkyl), —(C₁-C₆ alkylene)-NR^{1′}—SO₂—N(R^{1′})₂, —(C₁-C₆ alkylene)-S(O)₂—(C₁-C₄ alkyl), —(C₁-C₆ alkylene-S(O)₂—N(R^{1′})₂, or —(C₀-C₆ alkylene)-(C₃-C₈ cycloalkyl), wherein any alkyl, or alkylene portion of R¹ is optionally substituted with one or more independently selected monovalent substituents, any cycloalkyl portion of R¹ is optionally substituted with one or more independently selected substituents; and wherein each R^{1′} is, independently, hydrogen or optionally substituted C₁-C₆ alkyl (i.e., two R^{1′} cannot be taken together to form a ring).

In some embodiments, R¹ is hydrogen, cyclopropyl, —CH₃, —CH₂CH₃, —CH₂CH₂OCH₃, —CH(CH₃)₂, or —CH₂CH(CH₃)₂, or R¹ is taken together with one R² and the ring atoms to which each are attached to form a bridged ring which, taken together with the ring to which R¹ and R² are bound, forms

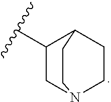 [SMILES: *C1CN2CCC1CC2]

In some embodiments, R¹ is hydrogen, —CH₃, or —CH₂CH₂OCH₃. In some embodiments, R¹ is hydrogen.

In some embodiments, each alkyl in any R^{1′} is optionally substituted with one or more substituents independently selected from fluorine, —OH and —CN.

In some embodiments, any heterocyclyl and heteroaryl rings formed from two R^{1′} are optionally substituted with one or more substituents independently selected from halo; C₁-C₄ alkyl; C₃-C₆ cycloalkyl optionally substituted with one or more substituents independently selected from fluorine, —OH and —CN; —OH; ═O; —CN; —C(O)NH₂; —C(O)NH(C₁-C₄ alkyl); —C(O)N(C₁-C₄ alkyl)₂; —S(O)₂—C₁-C₄-alkyl; —S(O)₂—NH₂; —S(O)₂—NH(C₁-C₄ alkyl); and —S(O)₂—N(C₁-C₄ alkyl)₂, wherein any alkyl portion of a substituent on any heterocyclyl and heteroaryl ring formed from two R^{1′} is optionally substituted with one or more further substituents independently selected from fluorine, —OH and —CN.

In some embodiments, any alkyl, alkylene, or aryl portion of R² is optionally substituted with one or more independently selected monovalent substituents. For example, any alkyl, alkylene, aryl, cycloalkyl, heterocyclyl or heteroaryl portion of R², any ring formed by taking R¹ together with R², or any ring formed by taking two R² together can be optionally substituted with one or more independently selected monovalent substituents.

In some embodiments, any heterocyclyl and heteroaryl portion of R² is optionally substituted with one or more substituents independently selected from halo, —C₁-C₄ alkyl, —OH, ═O, —CN, —C(O)N(R^{1′})₂, —C(O)OR^{1′}, —C(O)OH, —S(O)₂—(C₁-C₄-alkyl), —S(O)₂—N(R^{1′})₂; and any alkyl, alkylene and cycloalkyl portion of R² or a substituent thereon is optionally substituted with one or more substituents independently selected from fluorine, —OH and —CN.

In some embodiments, when two R² are taken together to form a ring, or R¹ and R² are taken together to form a ring, the resulting ring is optionally substituted with one or more substituents independently selected from halo, C₁-C₄ alkyl, —OH, ═O, —CN, —C(O)NR^{1′}₂, —S(O)₂—C₁-C₄-alkyl, —S(O)₂—N(R^{1′})₂; and any alkyl portion of a substituent on a ring formed when two R² are taken together to form a ring, or R¹ and R² are taken together is optionally substituted with one or more substituents independently selected from fluorine, —OH and —CN. In some embodiments, each R², if present, is independently selected from halo, —OH, —C₁-C₆ alkyl, —NHC(O)—(C₁-C₄ alkyl), —C(O)NH—C₁-C₄ alkyl, —C(O)— (optionally substituted heterocyclyl), optionally substituted aryl, and optionally substituted heteroaryl; or

R¹ and any R² are taken together with the atoms to which they are bound to form an optionally substituted heterocyclyl or heteroaryl ring fused, spirofused or bridged to the piperidine ring, or

two R² are taken together to form oxo (═O), or taken together with the atom or atoms to which they are bound and any intervening ring atoms to form an optionally substituted aryl, cycloalkyl, heterocyclyl or heteroaryl ring fused, spirofused or bridged to the piperidine ring,

wherein any alkyl or alkylene portion of R², any ring formed by taking R¹ together with R², or any ring formed by taking two R² together is optionally substituted with one or more independently selected monovalent substituents.

In some embodiments, each R², if present, is, independently, halo, ═O, —OH, optionally substituted —C₁-C₄ alkyl, optionally substituted phenyl or an optionally substituted heteroaryl. In some embodiments, each R² that is —C₁-C₄ alkyl or phenyl is optionally substituted with one or more independently selected monovalent substituents. In some embodiments, each R², if present, is, independently, halo or optionally substituted —C₁-C₄ alkyl. In some embodiments, each R², if present, is, independently, halo or —C₁-C₄ alkyl optionally substituted with one or more independently selected monovalent substituents. In some embodiments, each R², if present, is halo. In some embodiments, each R², if present, is optionally substituted —C₁-C₄ alkyl. In some embodiments, each R², if present, is —C₁-C₄ alkyl optionally substituted with one or more independently selected monovalent substituents.

In some embodiments, n is 0, 1, 2 or 3.

In some embodiments, n is 0, 1, 2 or 3, and each R², if present, is, independently, fluoro, —CH₃, —CH₂CH₃, —OH, or unsubstituted phenyl, or two R² are taken together to form oxo.

In some embodiments, n is 0, 1, 2 or 3, and each R², if present, is, independently, —CH(CH₃)₂, —C(O)NHCH₃, —NHC(O)CH₂CH₃, 3-methyl-1,2,4-oxadiazol-5-yl, 1,2,4-triazolo[4,3-a]pyridin-3-yl, 8-(methylsulfonyl)-1,2,4-triazolo[4,3-a]pyridin-3-yl, pyrrolidin-1-ylcarbonyl, or 3-hydroxypyrrolidin-1-ylcarbonyl; or two R² on different atoms are taken together with the atoms to which they are bound and any intervening ring atoms to form a ring which, taken together with the piperidine ring to which both R² are bound, is

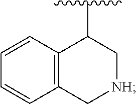 [SMILES: *C1CNCc2ccccc21]

or two R² bound to the same ring atom are taken together with the atom to which they are bound to form a ring which, taken together with the piperidine ring to which both R² are bound, is:

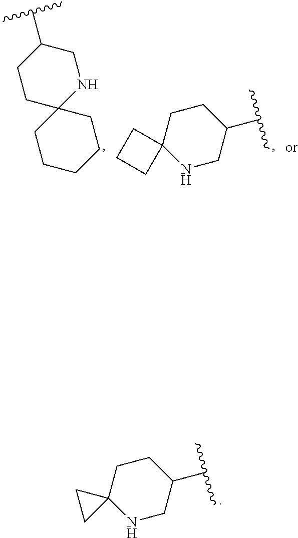

In some embodiments, each alkyl or cycloalkyl portion of R³ is optionally and independently substituted with one or more fluorine.

In some embodiments, R³ is hydrogen.

In some embodiments, any alkyl, alkenyl, alkynyl, or cycloalkyl portion of R⁴ is optionally and independently substituted with one or more substituents independently selected from —OH and fluorine.

In some embodiments, R⁴ is halo, —CN, optionally substituted C₁-C₄ alkyl, optionally substituted C₂-C₄ alkynyl, optionally substituted —O—C₁-C₄ alkyl, or optionally substituted C₃-C₆ cycloalkyl. In some embodiments, R⁴ is halo, —CN, optionally substituted C₁-C₄ alkyl, or optionally substituted C₁-C₄ haloalkyl. In some embodiments, R⁴ is halo, C₁-C₄ alkyl, or C₁-C₄ haloalkyl. In some embodiments, R⁴ is C₁-C₄ alkyl. In some embodiments, R⁴ is C₁-C₄ haloalkyl. In some embodiments, R⁴ is halo.

In some embodiments, R⁴ is hydrogen or —C(O)— (optionally substituted C₁-C₄ alkyl).

In some embodiments, R⁴ is chloro, fluoro, bromo, iodo, cyclopropyl, —CN, —CF₃, —CH₂CF₃, —CH₃, —CH₂CH₃, —CH(CH₃)₂, —CH₂CH(CH₃)₂, —OCH₃, or —C≡CH. In some embodiments, R⁴ is chloro, fluoro, —CF₃, —CH₂CF₃, —CH₃, —CH₂CH₃, or —C≡CH. In some embodiments, R⁴ is chloro, —CF₃, —CH₃, or —CH₂CH₃. In some embodiments, R⁴ is chloro or —CF₃. In some embodiments, R⁴ is chloro. In some embodiments, R⁴ is —CF₃.

In some embodiments, R⁴ is —CH₂CH₂F, —CH₂CH₂CH₃, —CH(OH)CH₃, —CH═CH₂, —C(O)CH₃, —OCHF₂, —S—CH₃, —S—CHF₂, or —S—CF₃.

In some embodiments, any heterocyclyl or heteroaryl portion of each R⁵ or a ring formed when two vicinal R⁵ are taken together is optionally and independently substituted with one or two substituents independently selected from halo, —CN, C₁-C₆ alkyl, —OH, ═O, —C(O)NR^{1′}₂, or —SO₂—NR^{1′}₂; and any alkyl, alkylene and cycloalkyl portions of R⁵, a substituent on R⁵, or a substituent on a ring formed by taking together two R⁵ is optionally substituted with one or more substituents independently selected from fluorine, —OH and —CN.

In some embodiments, one R⁵ is an optionally substituted heteroaryl or an optionally substituted heterocyclyl. For example, the heteroaryl or heterocyclyl is pyrazol-4-yl, imidazol-1-yl, morpholin-4-yl, pyridin-4-yl, pyridazin-4-yl, 1H-pyrrol-3-yl, pyridazin-4-yl, 1,2,4-triazol-3-yl, or 1,2,4-oxadiazol-3-yl; and is optionally substituted with one or two substituents selected from halo, —CN, C₁-C₆ alkyl, —OH, —C(O)N(R^{1′})₂, and —SO₂—N(R^{1′})₂.

In some embodiments, each R⁷ is, independently, hydrogen, halo, —C₁-C₆ alkyl, —CN, —C(O)OH, —C(O)—(C₁-C₄ alkyl), —C(O)—N(R^{1′})₂, —S(O)₂—(C₁-C₄ alkyl), —P(O)(C₁-C₄ alkyl)-O—C₁-C₄ alkyl, —P(O)(O—(C₁-C₄ alkyl))₂, heterocyclyl, or heteroaryl, wherein any alkyl, heterocyclyl or heteroaryl is optionally substituted.

In some embodiments, each R⁷ is, independently, —C(O)-heterocyclyl, —S(O)₂N(R^{1′})₂, —(C₁-C₄ alkylene)-S(O)₂—(C₁-C₄ alkyl), carbocyclyl, —O—(C₀-C₆-alkylene)-carbocyclyl, phenyl, —(C₂-C₄ alkenylene)-phenyl, —S(O)—(C₁-C₄ alkyl), —S—(C₁-C₄ alkyl), —S(O)—OH, or —S(O)₂—OH, wherein any alkyl, alkylene, alkenylene, carbocyclyl, phenyl, or heterocyclyl is optionally substituted.

In some embodiments, each R⁷ is, independently, hydrogen, fluoro, chloro, bromo, —CN, —CH₃, —CH₂CH₂C(CH₃)₂₀H, —C(O)—CH₃, —C(O)OH, —C(O)—NH—CH₃, —P(═O)(OCH₂CH₃)₂, —P(═O)(OCH₂CH₃)CH₃, —S(O)₂CH₃, 1H-pyrazol-4-yl, 1-methylpyrazol-4-yl, 1,3-dimethyl-pyrazol-4-yl, 5-methyl-1H-pyrazol-4-yl, 1-methyl-2-oxoimidazolidin-3-yl, 4-methylimidazol-1-yl, morpholin-4-yl, pyridin-4-yl, 4-hydroxycyclohexyl, 4-hydroxy-4-methylcyclohexyl, 5-methyl-1,2,4-triazol-3-yl, 5-methyl-1,2,4-oxadiazol-3-yl, 1,3-dimethylpyridazin-4-yl, 1,5-dimethylpyridazin-4-yl, 3-methyl-1H-pyridazin-4-yl, 1-(2-methyl-2-hydroxypropyl)pyridazin-4-yl, imidazol-1-yl, 1-methyl-5-cyanopyrrol-3-yl, 5-cyano-1H-pyrrol-3-yl, or pyridazin-4-yl.

In some embodiments, each R⁷ is, independently, —P(O)—(CH₃)₂, —P(O)—(CH₂CH₃)₂, —S(O)₂N(CH₃)₂, —S(O)₂CH(CH₃)₂, —S(O)₂CH₂F, —S(O)₂CHF₂, —SCHF₂, —S(O)CHF₂, —S(O)OH, —S(O)₂₀H, —S(O)₂NHCH₃, —(CH₂)₄CH₃, —CH₂S(O)₂CH₃, —S(O)₂—CH₂CH₃, 1H-pyrazol-3-yl, 1-difluoromethyl-pyrazol-3-yl, 1-difluoromethyl-pyrazol-4-yl, 1-methylpyrazol-3-yl, 3-methyl-1H-pyrazol-4-yl, 3-methyl-3-hydroxypyrrolidin-1-ylcarbonyl, 3-hydroxypyrrolidin-1-ylcarbonyl, 4-hydroxycyclohexyl, 4-hydroxycyclohex-1-enyl, 1,1-dioxothiomorpholin-4-yl, 4-cyano-1H-imidazol-1-yl, 2,3-dimethyl-1,2,4-triazol-5-yl, 1,5-dimethyl-pyrazol-4-yl, pyridin-3-yl, 1-(2-methyl-2-hydroxypropan-1-yl)pyrazol-4-yl, pyrrolidin-1-yl, pyrrolidin-1-ylcarbonyl, 1H-pyrazol-2-yl, 3-hydroxy-3-trifluoromethylpyrrolidin-1-ylcarbonyl, 3-methoxypyrrolidin-1-ylcarbonyl, 3-cyanopyrrolidin-1-ylcarbonyl, 4-hydroxy-4-methylpiperindin-1-ylcarbonyl, 3-oxopyrrolidin-1-ylcarbonyl, 3-(pyrrolidin-1-ylcarbonyl)phenyl, 3-phenoxyphenyl, thiazol-2-yl, pyrazin-2-yl, 2,4-dioxo-1H,3H-pyrimidin-5-yl, 3-methyl-3-hydroxypyrrolidin-1-ylsulfonyl, 5-flluoropyridin-3-yl, 2-hydroxpyridin-3-yl, 3,3-difluoro-4-hydroxy, 3,5-dimethyloxazol-4-yl, 3-fluorophenyl, 4-methylpyridin-3-yl, 2-hydroxymethylpyridin-3-yl, 6-hydroxymethylpyridin-2-yl, 5-hydroxymethylpyridin-3-yl, 1-methyl-6-oxopyridin-3-yl, 4-aminosulfonylphenyl, 3-aminosulfonylphenyl, 3-hydroxy-3-ethylpyrrolidin-1-ylcarbonyl, 3-cyano-4-hydroxyphenyl, benzo[d]thiazol-6-yl, 2H-indazol-6-yl, 1H-benzoimidazol-5-yl, 2-oxo-3-cyano-4-methylpyridin-5-yl, 2-aminobenzo[d]thiazol-2-yl, 3-aminocarbonylphenyl, 6-trifluoromethyl-1H-pyrrolo[3,2-c]pyridin-3-yl, 2-aminoquinazolin-8-yl, styryl, 1-methyl-TH-indazol-6-yl, 2,3-dihydrobenzo[b][1,4]dioxin-7-yl, 2-ethoxyphenyl, 3-(2-hydroxyethyl)phenyl, 3-(methylcarbonylaminomethyl)phenyl, 1-methyl-6-trifluoromethyl-1H-pyrrolo[3,2-c]pyridin-3-yl, quinolin-4-yl, isoquinolin-5-yl, isoquinolin-7-yl, or 2-oxo-3,4-dihydroquinolin-7-yl.

In some embodiments, each heterocyclyl and heteroaryl portion of R^{5′} is optionally substituted with one or more substituents independently selected from halo, C₁-C₄ alkyl, —OH, ═O, —CN, —C(O)NR^{1′}₂, and —SO₂—NR^{1′}₂, and each alkyl, alkylene and cycloalkyl portion of R^{5′} or a substituent of R^{5′} is optionally substituted with one or more substituents independently selected from fluorine, —OH and —CN.

In some embodiments, R^{5′} is hydrogen, C₁-C₄ alkyl, —(C₀-C₃ alkylene)-aryl or —(C₁-C₃ alkylene)-O—(C₁-C₄ alkyl). For example, R^{5′} can be hydrogen, methyl, isopropyl, —CH₂—O—CH₃, —(CH₂)₂—O—CH₃, or phenyl. In some embodiments, R⁶ is hydrogen or methyl.

In some embodiments, the compound of Formula (I) is a compound of Formula (I-a):

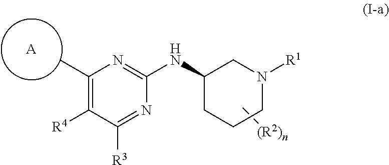

or a pharmaceutically acceptable salt thereof, wherein each of ring A, R¹, R², R³, R⁴, and n is defined as for Formula (I).

In some embodiments, the compound of Formula (I) is a compound of Formula (I-b):

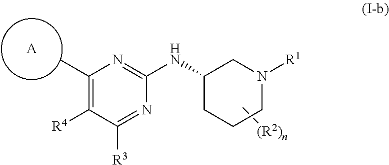

or a pharmaceutically acceptable salt thereof, wherein each of ring A, R¹, R², R³, R⁴, and n is defined as for Formula (I).

In some embodiments, the compound of Formula (I) is a compound of Formula (I-c):

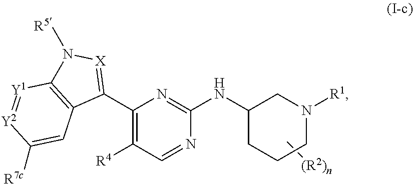

or a pharmaceutically acceptable salt thereof, wherein each of X, R², R⁴, R^{5′}, R⁷, R⁸, and n is defined as for Formula (I); Y¹ is N or C(R^{7a}); Y² is N or C(R^{7b}); and no more than one of X, Y¹ or Y² is N, wherein each of R^{7a}, R^{7b} and R^{7c} is independently selected from R⁷ as defined as for Formula (I).

In some embodiments, the compound of Formula (I-c) is a compound of Formula (I-c1):

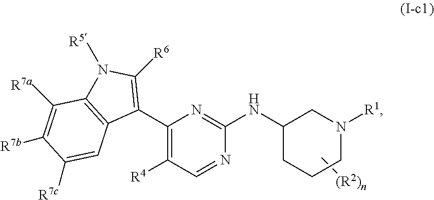

or a pharmaceutically acceptable salt thereof, wherein R⁶ is also as defined as for Formula (I).

In some embodiments, the compound of Formula (I-c) is a compound of Formula (I-c2):

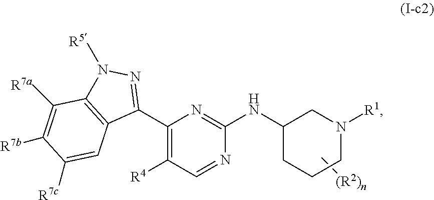

or a pharmaceutically acceptable salt thereof.

In some embodiments, the compound of Formula (I) is a compound of Formula (II):

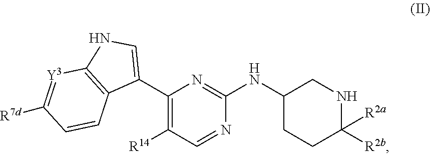

or a pharmaceutically acceptable salt thereof, wherein: Y³ is N or C(R^{7e});

each of R^{2a} and R^{2b} is, independently, hydrogen or C₁-C₃ alkyl; or

R^{2a} and R^{2b} are taken together to form a cycloalkyl or a heterocycle spirofused to the piperidine ring, wherein the cycloalkyl or heterocycle is optionally substituted with one or more independently selected C₁-C₄ alkyl or C₁-C₄ haloalkyl;

R^{7d} is hydrogen, —C(O)—(C₁-C₄ alkyl), —CN, or heteroaryl optionally substituted with one or more independently selected C₁-C₄ alkyl or C₁-C₄ haloalkyl;

R^{7e}, if present, is hydrogen, halo, —S(O)₂—(C₁-C₄ alkyl), —P(O)(C₁-C₄ alkyl)₂, —C(O)NH—(C₁-C₄ alkyl), —C(O)N(C₁-C₄ alkyl)₂, —S(O)₂NH—(C₁-C₄ alkyl), —S(O)₂N—(C₁-C₄ alkyl)₂, or heteroaryl optionally substituted with one or more independently selected C₁-C₄ alkyl or C₁-C₄ haloalkyl; and

R¹⁴ is C₁-C₃ alkyl or C₁-C₃ haloalkyl.

In some embodiments, the compound of Formula (II) is a compound of Formula (IIa):

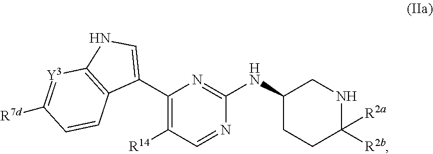

or a pharmaceutically acceptable salt thereof, wherein Y³, R^{2a}, R^{2b}, R^{7d}, R^{7e}, and R¹⁴ are as defined in Formula (II).

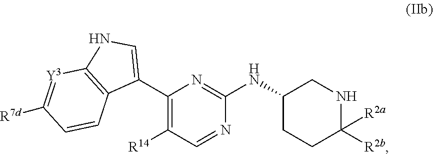

In some embodiments, the compound of Formula (I) is a compound of Formula (III):

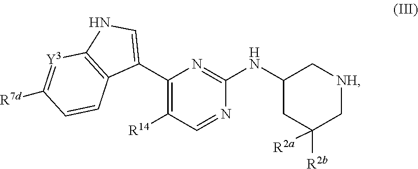

or a pharmaceutically acceptable salt thereof, wherein Y³, R^{2a}, R^{2b}, R^{7d}, R^{7e}, and R¹⁴ are as defined in Formula (II).

In some embodiments, the compound of Formula (III) is a compound of Formula (IIIa):

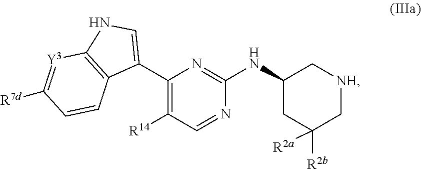

or a pharmaceutically acceptable salt thereof, wherein Y³, R^{2a}, R^{2b}, R^{7d}, R^{7e}, and R¹⁴ are as defined in Formula II.

In some embodiments, the compound of Formula (III) is a compound of Formula (IIIb):

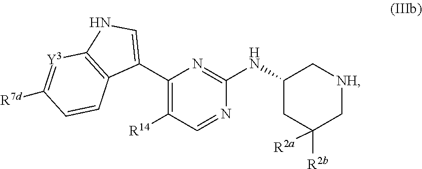

or a pharmaceutically acceptable salt thereof, wherein Y³, R^{2a}, R^{2b}, R^{7d}, R^{7e}, and R¹⁴ are as defined in Formula (II).

In some embodiments, in a compound of any one of Formulae (II), (IIa), (IIb), (III), (IIIa), or (IIIb):

R^{2a} is hydrogen or —CH₃;

R^{2b} is hydrogen, —CH₃, —CH₂CH₃, or —CH(CH₃)₂ or R^{2a} and R^{2b} are taken together to form oxetan-3-yl;

R^{7d} is hydrogen, —C(O)CH₃, —CN, pyridin-3-yl, pyridin-4-yl, 1-methyl-5-cyanopyrrol-3-yl, 1-methylpyrazol-4-yl, 1-methylpyrazol-3-yl, 1H-pyrazol-4-yl, 1H-pyrazol-3-yl, 1H-imidazol-2-yl, 1,3-dimethylpyrazol-4-yl, 1,5-dimethylpyrazol-4-yl, 1,5-dimethyl-1,2,4-triazol-3-yl, imidazol-1-yl, 1-difluoromethylpyrazol-3-yl, 1-difluoromethylpyrazol-4-yl, or thiazol-2-yl;

R^{7e}, if present, is hydrogen, fluoro, chloro, bromo, —CN, —P(O)(CH₃)₂, —S(O)₂CH(CH₃)₂, —S(O)₂CH₂CH₃, —S(O)₂N(CH₃)₂, —C(O)NHCH₃, pyridin-4-yl, pyridazin-4-yl, 5-methyl-1H-pyrazol-4-yl, 1-methylpyrazol-4-yl, 4-methyl-1H-imidazol-1-yl, 1H-benzo[d]imidazol-5-yl, 6-(trifluoromethyl)-1H-pyrrolo[3,2-c]pyridin-3-yl, 1-methyl-6-(trifluoromethyl)-1H-pyrrolo[3,2-c]pyridin-3-yl, isoquinolin-7-yl, isoquinolin-5-yl, pyrazin-2-yl, 2H-indazol-6-yl, 3,5-dimethylisoxazol-4-yl, thiazol-2-yl, 4-methylpyridin-3-yl, 1-methylindazol-6-yl, quinolin-4-yl, benzo[d]thiazol-6-yl, or 1,3-dimethylpyrazol-4-yl; and

R¹⁴ is —CH₃, —CF₃, —CH₂CH₃, —CH₂CF₃, —CH₂CH₂F, or —CH(CH₃)₂.

In some embodiments, in a compound of Formula (II), R^{2a} is hydrogen or —CH₃; R^{2b} is hydrogen or —CH₃; R^{7d} is hydrogen, —CN, pyrazin-2-yl, thiazol-2-yl, or 3,5-dimethylisoxazol-4-yl; R^{7e}, if present, is hydrogen, fluoro, —C(O)NHCH₃, —P(O)(CH₃)₂, —S(O)₂CH₃, —S(O)₂N(CH₃)₂, 1,3-dimethylpyrazol-4-yl, or pyridazin-4-yl; and R¹⁴ is —CH₂CH₃ or —CF₃.

In some embodiments, the compound of Formula (I) is any one of the compounds in the table of FIG. 1A-1X or is a pharmaceutically acceptable salt thereof.

The diagnostic and therapeutic methods described herein can also employ compounds related to pyrazolo[1,5-a]pyrimidine-5,7-diamine (i.e., such compounds can be administered to a patient identified for treatment in a manner described herein):

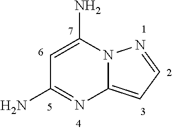 [SMILES: Nc1cc(N)n2nccc2n1]

and, to reiterate, all such compounds, including those of a subgenus described below, are encompassed by our references to “a CDK7 inhibitor described herein.” More specifically, related to 4-[[(7-aminopyrazolo[1,5-a]pyrimidin-5-yl)amino]methyl]piperidin-3-ol:

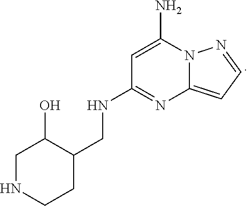 [SMILES: Nc1cc(NCC2CCNCC2O)nc2ccnn12]

Yet more specifically, the compound employed is a certain substituted 4-[[(7-aminopyrazolo[1,5-a]pyrimidin-5-yl)amino]methyl]piperidin-3-ol of Formula (IV) that has the following structural formula, wherein R¹⁵, R¹⁶, and R¹⁷ are as defined herein.

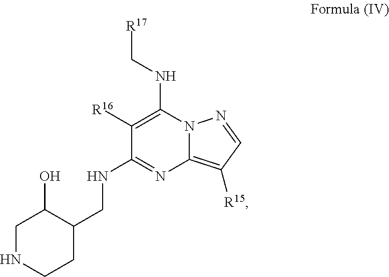

wherein R⁵ is hydrogen, C₁-C₆-alkyl (e.g., methyl) or C₃-C₆-cycloalkyl, each optionally substituted by 1-3 (e.g., 1 or 2) halogens (e.g., fluoro); R¹⁶ is hydrogen, halogen, C₁-C₆-alkyl, or C₁-C₆-haloalkyl; and R¹⁷ is phenyl optionally substituted with 1-3 (e.g., 1 or 2) substituents selected from halogen (e.g., fluoro), —CN, C₁-C₆-alkyl (e.g., methyl), C₃-C₆-cycloalkyl, and C₁-C₆-haloalkyl.

In some embodiments, R¹⁵ is hydrogen, C₁-C₆-alkyl or C₃-C₆-cycloalkyl. In some embodiments, R¹⁵ is hydrogen, ethyl, isopropyl, or cyclopropyl. In some embodiments, R¹⁶ is hydrogen or halogen. In some embodiments, R¹⁷ is phenyl optionally substituted with 1 substituent selected from the group consisting of halogen, —CN, C₁-C₆-alkyl, C₃-C₆-cycloalkyl, and C₁-C₆-haloalkyl. In some embodiments, R¹⁷ is phenyl optionally substituted with 1 substituent selected from halogen and —CN. In some embodiments, R¹⁵ is hydrogen, C₁-C₆-alkyl or C₃-C₆-cycloalkyl; R¹⁶ is hydrogen or halogen; and R¹⁷ is phenyl optionally substituted with 1 substituent selected from halogen and —CN.

Exemplary/useful compounds are shown in Table X of FIG. 2. The present diagnostic and/or therapeutic methods can be carried out with the following compound (ICEC0942), which is also “a CDK7 inhibitor described herein:”

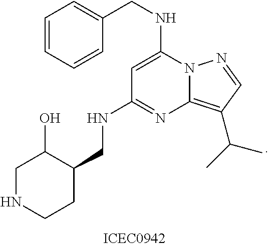 [SMILES: CC(C)c1cnn2c(NCc3ccccc3)cc(NC[C@H]3CCNCC3O)nc12]

The diagnostic and therapeutic methods described herein can also employ a compound of Formula (X) (i.e., such compounds can be administered to a patient identified for treatment in a manner described herein):

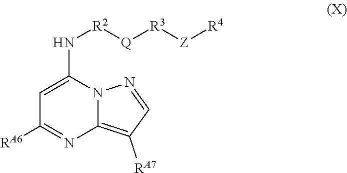

wherein: R^{A6} is C₁-C₆ alkyl; R^{A7} is C₁-C₆ alkyl; R² is a bond; Q is an optionally substituted divalent heteroaryl; R³ is C₁-C₄ alkylene; Z is a monocyclic heteroaryl; and R⁴ is Formula (ii-1):

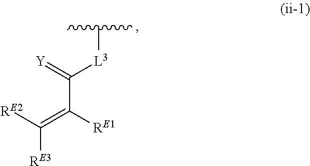

wherein L³ is a bond; Y is O, S, or N(R⁶) wherein R⁶ is hydrogen; R^{E1} is hydrogen; R^{E2} is hydrogen; and R^{E3} is CH₂N(R⁹)₂ wherein R⁹ is hydrogen or unsubstituted alkyl. In other embodiments, in a compound of Formula (X), R^{A6} is methyl; R^{A7} is a C₃ alkyl (e.g., a branched C₃ alkyl such as CH₂(CH₃)₂); Q is an unsubstituted divalent piperidine; R³ is a C₂ alkylene; Z is pyrrolyl; Y is O; and R⁹ is methyl. In one embodiment, R³ is a C₂ alkylene in which the first methylene unit is replaced with —O— and the second methylene unit is replaced with —C(O)—. In one embodiment, R^{A7} is a branched C₃ alkyl (CH₂(CH₃)₂) and R³ is a C₂ alkylene in which the first methylene unit is replaced with —O— and the second methylene unit is replaced with —C(O)—.
For example, the compound/CDK7 inhibitor can be

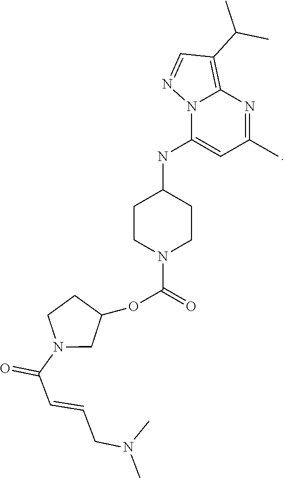 [SMILES: Cc1cc(NC2CCN(C(=O)OC3CCN(C(=O)/C=C/CN(C)C)C3)CC2)n2ncc(C(C)C)c2n1]

Other CDK7 inhibitors useful in the present methods are:

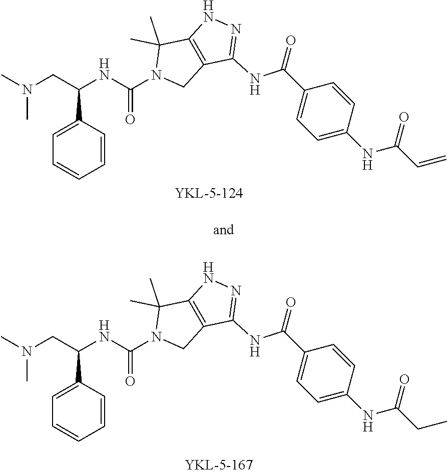

Information regarding the synthesis of YKL-5-124 and YKL-5-167 as well as other, similar compounds and guidance concerning, for example, dosages and indications, can be found in U.S. Application Publication No. 2019/0055248, the entire content of which is hereby incorporated by reference herein.

The diagnostic methods that identify a patient for treatment include a step of analyzing one or more of the biomarkers described herein in a biological sample obtained from the patient by determining, having determined, or receiving information concerning the state of the biomarker. In various embodiments, the state is assessed based on the presence, absence (e.g., a genetic deletion), location (e.g., chromosomal translocation), or copy number of a biomarker gene in wild type or mutant form, the inclusion of epigenetic modifications, the association of a biomarker gene with a super-enhancer (SE) or a SE of a certain strength, prevalence rank, or ordinal rank, the level of expression of the biomarker gene (as evidenced by, for example, its level of RNA (e.g., primary RNA transcripts or mRNA (e.g., pre-mRNA or mature mRNA)) expression), and/or the level of expression or activity of the protein encoded by the biomarker gene, each of which is discussed further below. One of ordinary skill in the art will understand that an RNA sequence can be reverse transcribed to generate a complimentary DNA (cDNA), and any of the methods and uses described herein can be carried out with cDNA that has been generated from an RNA described herein (i.e., a patient's biomarker status may have been determined using cDNA rather than an RNA). The state of a biomarker can be assessed in terms of any one or more of the features just listed regardless of the exact biomarker in use or the precise method or context in which the biomarker is being assessed. The state of a given biomarker (e.g., its copy number, associated enhancer, expression level, or activity) may be equal to or above a pre-determined threshold level or cutoff or equal to or below a pre-determined threshold level or cutoff, as described further below. In the methods of the present invention, one can analyze a biomarker selected from the genes BRAF, c-myc (also known as MYC), CDK1, CDK2, CDK4, CDK6, CDK17, CDK18, CDK19, CCNA1, CCNB1, CCNE1, ESR-1, FGFR1, PIC3CA, and certain genes encoding an E2F pathway member (see the Table below), or the proteins encoded thereby, by determining, having determined, and/or receiving information that the state of such a biomarker is equal to or above (e.g., above) a pre-determined threshold level (for RB1-E2F family members, see the Table below and the accompanying teaching). Alternatively, or in addition, one can analyze a biomarker selected from the genes BCL2-like 1, CDK7, CDK9, CDKN2A, and RB (also known as RB1 or another E2F pathway member), and/or the proteins encoded thereby, by determining, having determined, and/or receiving information that the state of such biomarker is equal to or below (e.g., below) a pre-determined threshold level (for RB/E2F family members, see the Table below). The choice of which biomarker(s) to utilize may depend, in part, on the particular cancer afflicting the patient, as well as other factors described herein. CDK18 encodes CDK18, and CDK19 encodes CDK19 (a component of the Mediator co-activator complex); CCNE1 encodes cyclin E1 (see Koff et al., Cell 66:1217-1228, 1991); FGFR1 encodes FGFR1, a cell surface membrane receptor with tyrosine kinase activity; RB encodes pRB, which binds to the activator domain of activator E2F; BCL2-like 1 encodes BCL-xL, a transmembrane protein in mitochondria; CDK7 encodes CDK7; CDK9 encodes CDK9; and CDKN2A encodes p16 and p14arf. Aliases, chromosomal locations, splice variants, and homologs of the genes and proteins described herein as biomarkers, in species other than Homo sapiens, are known in the art.

The treatment methods of the invention and corresponding “uses” include administering, or the use of, a CDK7 inhibitor described herein (e.g., a compound of Formula (I)), any of which may be included in a pharmaceutically acceptable composition and administered by a route and regimen (e.g., as described further herein), to a patient identified as described herein (e.g., as having a type of cancer described herein).

### BRIEF DESCRIPTION OF THE DRAWINGS

FIG. 1A-1X is a table of exemplary/useful compounds of Formula (I). Additional exemplary/useful compounds are shown in Table X immediately below.

FIG. 2 is a table (Table X) illustrating additional CDK7 inhibitors useful in the methods of the invention.

### DETAILED DESCRIPTION

We believe the efficacy of a CDK7 inhibitor as described herein or a pharmaceutically acceptable salt thereof will be higher in patients that have certain genetic signatures (i.e., one or more biomarkers in a particular state, as also described herein). Moreover, we believe the efficacy of these compounds and their salts may be enhanced when combined with other anti-cancer therapies, as described herein, in patients, including those having a cancer described herein and/or identified by virtue of a biomarker as described herein.

The following definitions apply to the compositions, methods, and uses described herein unless the context clearly indicates otherwise, and it is to be understood that the claims may be amended to include language within a definition as needed or desired. Moreover, the definitions apply to linguistic and grammatical variants of the defined terms (e.g., the singular and plural forms of a term), and some linguistic variants are particularly mentioned below (e.g., “administration” and “administering”). The chemical elements are identified in accordance with the Periodic Table of the Elements, CAS version, Handbook of Chemistry and Physics, 75^{th} Ed. Additionally, general principles of organic chemistry are well established and one of ordinary skill in the art can consult, if desired, Organic Chemistry by Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The term “about,” when used in reference to a value, signifies any value or range of values that is plus-or-minus 10% of the stated value (e.g., within plus-or-minus 1%, 2%, 3%, 4%, 5%, 6%, 7%, 8%, 9% or 10% of the stated value). For example, a dose of about 10 mg means any dose as low as 10% less than 10 mg (9 mg), any dose as high as 10% more than 10 mg (11 mg), and any dose or dosage range therebetween (e.g., 9-11 mg; 9.1-10.9 mg; 9.2-10.8 mg; and so on). As another example, a prevalence rank in a population of about 80% means a prevalence rank of 72-88% (e.g., 79.2-80.8%). In case of doubt, “about X” can be “X” (e.g., about 80% can be 80%). Where a stated value cannot be exceeded (e.g., 100%), “about” signifies any value or range of values that is up to and including 10% less than the stated value (e.g., a purity of about 100% means 90%-100% pure (e.g., 95%-100% pure, 96%-100% pure, 97%-100% pure etc. . . . )). In the event an instrument or technique measuring a value has a margin of error greater than 10%, a given value will be about the same as a stated value when they are both within the margin of error for that instrument or technique.

The term “administration” and variants thereof, such as “administering,” refer to the administration of a CDK7 inhibitor described herein, including a compound conforming to a Formula disclosed herein or a pharmaceutically acceptable salt thereof, an additional/second agent, or a composition containing one or more of any such compounds to a subject (e.g., a human patient) or system (e.g., a cell- or tissue-based system that is maintained ex vivo); as a result of the administration, the compound or composition containing the compound (e.g., a pharmaceutical composition) is introduced to the subject or system. In addition to compositions of the invention and second agents useful in combination therapies, items used as positive controls, negative controls, and placebos, any of which can also be a compound, can also be “administered.” One of ordinary skill in the art will be aware of a variety of routes that can, in appropriate circumstances, be utilized for administration to a subject or system. For example, the route of administration can be oral (i.e., by swallowing a pharmaceutical composition) or may be parenteral. More specifically, the route of administration can be bronchial (e.g., by bronchial instillation), by mouth (i.e., oral), dermal (which may be or comprise topical application to the dermis or intradermal, interdermal, or transdermal administration), intragastric or enteral (i.e., directly to the stomach or intestine, respectively), intramedullary, intramuscular, intranasal, intraperitoneal, intrathecal, intratumoral, intravenous (or intra-arterial), intraventricular, by application to or injection into a specific organ (e.g., intrahepatic), mucosal (e.g., buccal, rectal, sublingual, or vaginal), subcutaneous, tracheal (e.g., by intratracheal instillation), or ocular (e.g., topical, subconjunctival, or intravitreal). Administration can involve intermittent dosing (i.e., doses separated by various times) and/or periodic dosing (i.e., doses separated by a common period of time (e.g., every so many hours, daily (e.g., once daily oral dosing), weekly, twice per week, etc.)). In other embodiments, administration may involve continuous dosing (e.g., perfusion) for a selected time (e.g., about 1-2 hours).

Two events, two entities, or an event and an entity are “associated” with one another if one or more features of the first (e.g., its presence, level and/or form) are correlated with a feature of the second. For example, a first entity (e.g., an enzyme (e.g., CDK7)), gene expression profile, genetic signature (i.e., a single or combined group of genes in a cell with a uniquely characteristic pattern of gene expression), metabolite, or event (e.g., myeloid infiltration)) is associated with an event (e.g., the onset or progression of a particular disease), if its presence, level and/or form correlates with the incidence of, severity of, and/or susceptibility to the disease (e.g., a cancer disclosed herein). The biomarkers described herein are associated with an identified patient in the manner described herein (e.g., by virtue of their level of expression). Associations are typically assessed across a relevant population. Two or more entities are physically “associated” with one another if they interact, directly or indirectly, so that they are and/or remain in physical proximity with one another in a given circumstance (e.g., within a cell maintained under physiological conditions (e.g., within cell culture) or within a pharmaceutical composition). Entities that are physically associated with one another can be covalently linked to one another or non-covalently associated by, for example, hydrogen bonds, van der Waals forces, hydrophobic interactions, magnetism, or combinations thereof. A CDK7 inhibitor described herein or a pharmaceutically acceptable salt thereof can be non-covalently associated with CDK7.

The term “biological sample” refers to a sample obtained or derived from a biological source of interest (e.g., a tissue or organism (e.g., an animal or human patient) or cell culture). For example, a biological sample can be a sample obtained from an individual (e.g., a patient or an animal model) suffering from a disease (or, in the case of an animal model, a simulation of that disease in a human patient) to be diagnosed and/or treated by the methods of this invention or from an individual serving in the capacity of a reference or control (or whose sample contributes to a reference standard or control population). The biological sample can contain a biological cell, tissue or fluid or any combination thereof. For example, a biological sample can be or can include ascites; blood; blood cells; a bodily fluid, any of which may include or exclude cells (e.g., tumor cells (e.g., circulating tumor cells (CTCs) found in at least blood or lymph vessels)); bone marrow or a component thereof (e.g., hematopoietic cells, marrow adipose tissue, or stromal cells); cerebrospinal fluid (CSF); feces; flexural fluid; free-floating nucleic acids (e.g., circulating tumor DNA); gynecological fluids; immune infiltrates; lymph; peritoneal fluid; plasma; saliva; sputum; surgically-obtained specimens; tissue scraped or swabbed from the skin or a mucus membrane (e.g., in the nose, mouth, or vagina); tissue or fine needle biopsy samples; urine; washings or lavages such as a ductal lavage or broncheoalveolar lavage; or other body fluids, tissues, secretions, and/or excretions. Samples of, or samples obtained from, a bodily fluid (e.g., blood, CSF, lymph, plasma, or urine) may include tumor cells (e.g., CTCs) and/or free-floating or cell-free nucleic acids of the tumor. Cells (e.g., cancer cells) within the sample may have been obtained from an individual patient for whom a treatment is intended. Samples used in the form in which they were obtained may be referred to as “primary” samples, and samples that have been further manipulated (e.g., by removing one or more components of the sample) may be referred to as “secondary” or “processed” samples. Such processed samples may contain or be enriched for a particular cell type (e.g., a CDK7-expressing cell, which may be a tumor cell), cellular component (e.g., a membrane fraction), or cellular material (e.g., one or more cellular proteins, including CDK7, DNA, or RNA (e.g., mRNA (e.g., pre-mRNA or mature mRNA)), which may encode CDK7 and may be subjected to amplification). As used herein, the term “biomarker” refers to an entity whose state correlates with a particular biological event so that it is considered to be a “marker” for that event (e.g., the presence of a particular cancer and/or its susceptibility to a CDK7 inhibitor described herein or a pharmaceutically acceptable salt thereof). A biomarker can be analyzed at the nucleic acid or protein level; at the nucleic acid level, one can analyze the presence (e.g., copy number alterations (CNAs)), absence, or chromosomal location of a gene in wild type or mutant form, epigenetic alterations (in, e.g., methylation), its association with a super-enhancer, and/or its level of expression (as evidenced, for example, by primary RNA transcript or mRNA (e.g., pre-mRNA or mature mRNA) levels). At the protein level, one can analyze the level of expression and/or activity of a protein encoded by a biomarker gene. A biomarker may indicate a therapeutic outcome or likelihood (e.g., increased likelihood) thereof. Thus, a biomarker can be predictive or prognostic and is therefore useful in methods of identifying or treating a patient as described herein.

The term “cancer” refers to a disease in which biological cells exhibit an aberrant growth phenotype characterized by loss of control of cell proliferation to an extent that will be detrimental to a patient having the disease. A cancer can be classified by the type of tissue in which it originated (histological type) and/or by the primary site in the body in which the cancer first developed. Based on histological type, cancers are generally grouped into six major categories: carcinomas; sarcomas; myelomas; leukemias; lymphomas; and mixed types. A cancer treated as described herein may be of any one of these types and may comprise cells that are precancerous (e.g., benign), malignant, pre-metastatic, metastatic, and/or non-metastatic. A patient who has a malignancy or malignant lesion has a cancer. The present disclosure specifically identifies certain cancers to which its teachings may be particularly relevant, and one or more of these cancers may be characterized by a solid tumor or by a hematologic tumor, which may also be known as a blood cancer (e.g., of a type described herein). Although not all cancers manifest as solid tumors, we may use the terms “cancer cell” and “tumor cell” interchangeably to refer to any malignant cell.

The term “combination therapy” refers to those situations in which a subject is exposed to two or more therapeutic regimens (e.g., two or more therapeutic agents) to treat a single disease (e.g., a cancer). The two or more regimens/agents may be administered simultaneously or sequentially. When administered simultaneously, a dose of the first agent and a dose of the second agent are administered at about the same time, such that both agents exert an effect on the patient at the same time or, if the first agent is faster- or slower-acting than the second agent, during an overlapping period of time. When administered sequentially, the doses of the first and second agents are separated in time, such that they may or may not exert an effect on the patient at the same time. For example, the first and second agents may be given within the same hour or same day, in which case the first agent would likely still be active when the second is administered. Alternatively, a much longer period of time may elapse between administration of the first and second agents, such that the first agent is no longer active when the second is administered (e.g., all doses of a first regimen are administered prior to administration of any dose(s) of a second regimen by the same or a different route of administration, as may occur in treating a refractory cancer). For clarity, combination therapy does not require that two agents be administered together in a single composition or at the same time, although in some embodiments, two or more agents, including a CDK7 inhibitor described herein or a pharmaceutically acceptable salt thereof and a second agent described herein may be administered within the same period of time (e.g., within the same hour, day, week, or month).

The terms “cutoff” and “cutoff value” mean a value measured in an assay that defines the dividing line between two subsets of a population (e.g., likely responders and non-responders (e.g., responders and non-responders to a CDK7 inhibitor described herein or a pharmaceutically acceptable salt thereof). In some instances, values that are equal to or above the cutoff value define one subset of the population, and values that are lower than the cutoff value define the other subset of the population. In other instances, values that are equal to or below the cutoff value define on subset of the population, and values above the cutoff value define the other. As described further below, the cutoff or cutoff value can define the threshold value.

As used herein, “diagnostic information” is information that is useful in determining whether a patient has a disease and/or in classifying (stratifying) the disease into a genotypic or phenotypic category or any category having significance with regard to the prognosis of the disease or its likely response to treatment (either treatment in general or any particular treatment described herein). Similarly, “diagnosis” refers to obtaining or providing any type of diagnostic information, including, but not limited to, whether a patient is likely to have or develop a disease; whether that disease has or is likely to reach a certain state or stage or to exhibit a particular characteristic (e.g., resistance to a therapeutic agent); information related to the nature or classification of a tumor; information related to prognosis (which may also concern resistance); and/or information useful in selecting an appropriate treatment (e.g., selecting a CDK7 inhibitor described herein or a pharmaceutically acceptable salt thereof for a patient identified as having a cancer that is likely to respond to such an inhibitor or other treatment). A patient classified (stratified) according to a method described herein and selected for treatment with a CDK7 inhibitor described herein or a pharmaceutically acceptable salt thereof is likely to respond well to the treatment, meaning that such a patient is more likely to be successfully treated than a patient with the same type of cancer who has not been so identified and is not in the same strata. Available treatments include therapeutic agents and other treatment modalities such as surgery, radiation, etc., and selecting an appropriate treatment encompasses the choice of withholding a particular therapeutic agent; the choice of a dosing regimen; and the choice of employing a combination therapy. Diagnostic information can be used to stratify patients and is thus useful in identifying and classifying a given patient according to, for example, biomarker status. Obtaining diagnostic information can constitute a step in any of the patient stratification methods described herein.

One of ordinary skill in the art will appreciate that the term “dosage form” may be used to refer to a physically discrete unit of an active agent (e.g., a therapeutic or diagnostic agent) for administration to a patient. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular patient is determined by one or more attending physicians and may involve administration of multiple dosage forms.

One of ordinary skill in the art will appreciate that the term “dosing regimen” may be used to refer to a set of unit doses (typically more than one) that are administered individually to a patient, separated by equal or unequal periods of time. A given therapeutic agent typically has a recommended dosing regimen, which may involve one or more doses, each of which may contain the same unit dose amount or differing amounts. In some embodiments, a dosing regimen comprises a first dose in a first dose amount, followed by one or more additional doses in a second dose amount that is different from the first dose amount. In some embodiments, a dosing regimen is correlated with a desired or beneficial outcome when administered across a relevant population (i.e., the regimen is a therapeutic dosing regimen).

As used herein, an “effective amount” of an agent (e.g., a chemical compound described herein), such as a compound of Formula (I) or other CDK7 inhibitor described herein, refers to an amount that produces or is expected to produce the desired effect for which it is administered. The effective amount will vary depending on factors such as the desired biological endpoint, the pharmacokinetics of the compound administered, the condition being treated, the mode of administration, and characteristics of the patient, as discussed further below and recognized in the art. The term can be applied to therapeutic and prophylactic methods. For example, a therapeutically effective amount is one that reduces the incidence and/or severity of one or more signs or symptoms of the disease. For example, in treating a cancer, an effective amount may reduce the tumor burden, inhibit tumor growth, inhibit metastasis or prolong patient survival. One of ordinary skill in the art will appreciate that the term does not in fact require successful treatment be achieved in any particular individual. Rather, a therapeutically effective amount is that amount that provides a particular desired pharmacological response in a significant number of patients when administered to patients in need of such treatment. In some embodiments, reference to a therapeutically effective amount may be a reference to an amount administered or an amount measured in one or more specific tissues (e.g., a tissue affected by the disease) or fluids (e.g., blood, saliva, serum, sweat, tears, urine, etc.). Effective amounts may be formulated and/or administered in a single dose or in a plurality of doses (e.g., as part of a dosing regimen).

As used herein, an “enhancer” is a region of genomic DNA that helps regulate the expression of genes up to 1 Mbp or so away. An enhancer may overlap, but is often not composed of, gene coding regions. An enhancer is often bound by transcription factors and designated by specific histone marks.

An “mRNA” is a single-stranded RNA product synthesized by transcription of DNA that includes one or more of the coding sequences of a gene. The mRNA may be in the form of a precursor (pre-mRNA) or may be further processed to a mature form of mRNA lacking introns.

The term “patient” refers to any organism that is or may be subjected to a diagnostic method described herein or to which a compound described herein or a pharmaceutically acceptable salt thereof, is or may be administered for, e.g., experimental, diagnostic, prophylactic, and/or therapeutic purposes. Typical patients include animals (e.g., mammals such as mice, rats, rabbits, non-human primates, and humans; domesticated animals, such as dogs and cats; and livestock or any other animal of agricultural or commercial value). A patient may be suffering from or susceptible to (i.e., have a higher than average risk of developing) a disease described herein and may display one or more signs or symptoms thereof.

The term “pharmaceutically acceptable,” when applied to a carrier used to formulate a composition disclosed herein (e.g., a pharmaceutical composition), means a carrier that is compatible with the other ingredients of the composition and not deleterious to a patient (e.g., it is non-toxic in the amount required and/or administered (e.g., in a unit dosage form)).

The term “pharmaceutically acceptable,” when applied to a salt, refers to a salt form of a compound that is, within the scope of sound medical judgment, suitable for use in contact with the tissues of humans (e.g., patients) and lower animals (including, but not limited to, mice and rats used in laboratory studies) without unacceptable toxicity, irritation, allergic response and the like, and that can be used in a manner commensurate with a reasonable benefit/risk ratio. Many pharmaceutically acceptable salts are well known in the art (see, e.g., Berge et al., J. Pharm. Sci. 66:1-19, 1977). Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, MALATle, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “population” means some number of items (e.g., at least 30, 40, 50, or more) sufficient to reasonably reflect the distribution, in a larger group, of the value being measured in the population. Within the context of the present invention, the population can be a discrete group of humans, laboratory animals, or cells lines (for example) that are identified by at least one common characteristic for the purposes of data collection and analysis. For example, a “population of samples” refers to a plurality of samples that is large enough to reasonably reflect the distribution of a value (e.g., a value related to the state of a biomarker) in a larger group of samples. The items in the population may be biological samples, as described herein. For example, each sample in a population of samples may be cells of a cell line or a biological sample obtained from a patient or a xenograft (e.g., a tumor grown in a mouse by implanting a tumorigenic cell line or a patient sample into the mouse). As noted, individuals within a population can be a discrete group identified by a common characteristic, which can be the same disease (e.g., the same type of cancer), whether the sample is obtained from living beings suffering from the same type of cancer or a cell line or xenograft representing that cancer.

The term “prevalence cutoff,” as used herein in reference to a specified value (e.g., the strength of a SE associated a biomarker disclosed herein) means the prevalence rank that defines the dividing line between two subsets of a population (e.g., a subset of “responders” and a subset of “non-responders,” which, as the names imply include patients who are likely or unlikely, respectively, to experience a beneficial response to a therapeutic agent or agents). Thus, a prevalence rank that is equal to or higher (e.g., a lower percentage value) than the prevalence cutoff defines one subset of the population; and a prevalence rank that is lower (e.g., a higher percentage value) than the prevalence cutoff defines the other subset of the population.

As used herein, the term “prevalence rank” for a specified value (e.g., the mRNA level of a specific biomarker) means the percentage of a population that are equal to or greater than that specific value. For example, a 35% prevalence rank for the amount of mRNA (e.g., pre-mRNA or mature mRNA) of a specific biomarker in a test cell means that 35% of the population have that level of biomarker mRNA or greater than the test cell.

A “primary RNA transcript” is a single-stranded ribonucleic acid (RNA) product synthesized by transcription of DNA and processed to yield various mature RNA products such as mRNAs, tRNAs, and rRNAs. The primary RNA transcripts designated as mRNAs are transcribed from DNA sequences that include one or more gene coding regions (exons) and may include sequence transcribed from a regulatory region (e.g., an enhancer or super-enhancer) associated with the gene. These primary RNA transcripts are modified in preparation for translation. A precursor mRNA (pre-mRNA) is the first form of RNA created through transcription, and it is modified to become the mature mRNA that lacks introns.

As used herein, the terms “prognostic information” and “predictive information” are used to refer to any diagnostic information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Diagnostic information can be prognostic or predictive.

As used herein, the term “rank ordering” means the ordering of values from highest to lowest or from lowest to highest.

As used herein, the terms “RB-E2F pathway” and “RB-E2F family” refer to a set of genes and the proteins encoded thereby, as the context will make clear, whose expression or activity regulates the activity of the RB gene family and in turn regulates the activity of the E2F family of transcription factors that are required for entry into and progression through the cell cycle. The table below contains a list of genes in the RB-E2F family, an indication of a currently understood function of the encoded proteins, and the status of these biomarkers in cancer. We use the shorthand “activated or overexpressed” to indicate that an attribute of a gene (e.g., its copy number or level of expression) or the protein it encodes (e.g., its level of expression or activity) is higher in some patients with certain cancers than it is in healthy subjects. A pre-determined threshold for such activated or overexpressed RB-E2F family members can be determined by comparative analysis and is a level (e.g., mRNA level (e.g., pre-mRNA or mature mRNA), protein level, gene copy number, strength of enhancer associated with the gene) that, when found or exceeded in a cancer patient, identifies that patient as a candidate for treatment as described herein. We use the shorthand “inactivated or underexpressed” to indicate that an attribute of a gene (e.g., its copy number, or level of expression) or a protein it encodes (e.g., its level of expression or activity) is lower in some patients with certain cancers than it is in healthy subjects. A pre-determined threshold for such inactivated or underexpressed RB-E2F family members can be determined by comparative analysis and is a level (e.g., mRNA level, protein level, gene copy number, strength of enhancer associated with the gene) that, when unattained in a cancer patient, identifies that patient as a candidate for treatment as described herein.

| Gene | Function | Status in Cancer |
|---|---|---|
| E2F1 | E2F family-transcriptional control of cell cycle entry | Activated or overexpressed |
| E2F2 | E2F family-transcriptional control of cell cycle entry | Activated or overexpressed |
| E2F3 | E2F family-transcriptional control of cell cycle entry | Activated or overexpressed |
| E2F4 | E2F family-transcriptional control of cell cycle entry | Activated or overexpressed |
| E2F5 | E2F family-transcriptional control of cell cycle entry | Activated or overexpressed |
| E2F6 | E2F family-transcriptional control of cell cycle entry | Activated or overexpressed |
| E2F7 | E2F family-transcriptional control of cell cycle entry | Activated or overexpressed |
| E2F8 | E2F family-transcriptional control of cell cycle entry | Activated or overexpressed |
| RB1 | RB family-E2F family inhibition | Inactivated or underexpressed |
| RBL1 | RB family-E2F family inhibition | Inactivated or underexpressed |
| RBL2 | RB family-E2F family inhibition | Inactivated or underexpressed |
| CDK4 | RB family inhibition | Activated or overexpressed |
| CDK6 | RB family inhibition | Activated or overexpressed |
| CDK2 | RB family inhibition | Activated or overexpressed |
| CCND1 | CDK4/6 regulation | Activated or overexpressed |
| CCND2 | CDK4/6 regulation | Activated or overexpressed |
| CCND3 | CDK4/6 regulation | Activated or overexpressed |
| CDKN2A | CDK4/6 regulation | Inactivated or underexpressed |
| CDKN2B | CDK4/6 regulation | Inactivated or underexpressed |
| CDKN2C | CDK4/6 regulation | Inactivated or underexpressed |
| CDKN2D | CDK4/6 regulation | Inactivated or underexpressed |
| CCNE1 | CDK2 regulation | Activated or overexpressed |
| CCNE2 | CDK2 regulation | Activated or overexpressed |
| CDKN1A | CDK2 regulation | Inactivated or underexpressed |
| CDKN1B | CDK2 regulation | Inactivated or underexpressed |
| CDKN1C | CDK2 regulation | Inactivated or underexpressed |
| FBXW7 | CCNE regulation | Inactivated or underexpressed |

As used herein, a “reference” refers to a standard or control relative to which a comparison is performed. For example, an agent, patient, population, sample, sequence, or value of interest is compared with a reference agent, patient, population, sample, sequence or value. The reference can be analyzed or determined substantially simultaneously with the analysis or determination of the item of interest or it may constitute a historical standard or control, determined at an earlier point in time and optionally embodied in a tangible medium. One of ordinary skill in the art is well trained in selecting appropriate references, which are typically determined or characterized under conditions that are comparable to those encountered by the item of interest. One will appreciate when sufficient similarities are present to justify reliance on and/or comparison to a particular possible reference as a standard or control.

As used herein, a “response” to treatment is any beneficial alteration in a patient's condition that results from, or that correlates with, treatment. The alteration may be stabilization of the condition (e.g., inhibition of deterioration that would have taken place in the absence of the treatment), amelioration of, delay of onset of, and/or reduction in frequency of one or more signs or symptoms of the condition, improvement in the prospects for cure of the condition, greater survival time, and etc. A response may be a patient's response or a tumor's response.

As used herein, when the term “strength” is used to refer to a portion of an enhancer or a SE, it means the area under the curve of the number of H3K27Ac or other genomic marker reads plotted against the length of the genomic DNA segment analyzed. Thus, “strength” is an integration of the signal resulting from measuring the mark at a given base pair over the span of the base pairs defining the region being chosen to measure.

As used herein, the term “super-enhancer” (SE) refers to a subset of enhancers that contain a disproportionate share of histone marks and/or transcriptional proteins relative to other enhancers in a particular cell or cell type. Genes regulated by SEs are predicted to be of high importance to the function of a cell. SEs are typically determined by rank ordering all of the enhancers in a cell based on strength and determining, using available software such as ROSE (bitbucket.org/young_computation/rose), the subset of enhancers that have significantly higher strength than the median enhancer in the cell (see, e.g., U.S. Pat. No. 9,181,580, which is hereby incorporated by reference herein in its entirety).

The terms “threshold” and “threshold level” mean a level that defines the dividing line between two subsets of a population (e.g., responders and non-responders). A threshold or threshold level can define a prevalence cutoff or a cutoff value.

As used herein, the terms “treatment,” “treat,” and “treating” refer to reversing, alleviating, delaying the onset of, and/or inhibiting the progress of a “pathological condition” (e.g., a disease, such as cancer) described herein. In some embodiments, “treatment,” “treat,” and “treating” require that signs or symptoms of the disease have developed or have been observed. In other embodiments, treatment may be administered in the absence of signs or symptoms of the disease or condition (e.g., in light of a history of symptoms and/or in light of genetic or other susceptibility factors). Treatment may also be continued after symptoms have resolved, for example, to delay or inhibit recurrence.

As the invention relates to compositions and methods for diagnosing and treating patients who have cancer, the terms “active agent,” “anti-cancer agent,” “pharmaceutical agent,” and “therapeutic agent” are used interchangeably (unless the context clearly indicates otherwise) and the CDK7 inhibitors described herein and pharmaceutically acceptable salts thereof would be understood by one of ordinary skill in the art as active, anti-cancer, pharmaceutical, or therapeutic agents. As noted, the treatment methods and uses encompass combination therapies/uses in which a CDK7 inhibitor described herein or a pharmaceutically acceptable salt thereof is administered or used in combination with one or more additional agents (e.g., an additional anti-cancer therapeutic), as described herein. In keeping with convention, in any embodiment requiring two agents, we may refer to one as the “first” agent and to the other as the “second” agent to underscore that the first and second agents are distinct from one another.

The invention also features kits that include a CDK7 inhibitor as described herein or a pharmaceutically acceptable salt thereof and instructional materials that describe a suitable/identified patient, methods of identifying such a patient for treatment (e.g., by any one of the diagnostic stratification methods described herein), and/or instructions for administering the CDK7 inhibitor alone or in combination with at least one other therapeutic agent (e.g., an additional/second anti-cancer therapeutic). The kits of the invention can also include a second agent (e.g., an anti-cancer agent), including any one or more of the second agents described herein and instructions for use in a population of patients identified as described.

As indicated, each therapeutic method and any diagnostic method that employs a CDK7 inhibitor described herein or a pharmaceutically acceptable salt thereof may also be expressed in terms of use and vice versa. For example, the invention encompasses the use of a compound or composition described herein for the treatment of a disease described herein (e.g., cancer); a compound or composition for use in diagnosing and/or treating or a disease (e.g., cancer); and the use of the compound or composition for the preparation of a medicament for treating a disease described herein (e.g., cancer).

The methods of the invention that concern diagnosing and/or treating a cancer described herein (or use of a CDK7 inhibitor for such purpose) may specifically exclude any one or more of the types of cancers described herein. For example, the invention features methods of treating cancer by administering a CDK7 inhibitor described herein or a pharmaceutically acceptable salt thereof with the proviso that the cancer is not a breast cancer; with the proviso that the cancer is not a breast cancer or a leukemia; with the proviso that the cancer is not a breast cancer, a leukemia, or an ovarian cancer; and so forth, with exclusions selected from any of the diseases listed herein and with the same notion of variable exclusion from lists of elements relevant to other aspects of the invention (e.g., chemical substituents of a compound described herein or components of kits and pharmaceutical compositions).

In one aspect, the invention features the use of a CDK7 inhibitor described herein or a pharmaceutically acceptable salt thereof in treating cancer in a patient who has been identified by virtue of having: (a) a level of BCL2-like 1 RNA (e.g., a primary RNA transcript or mRNA (e.g., pre-mRNA or mature mRNA) encoding BCL-xL) in a biological sample including cancer cells obtained from the patient, the level being equal to or below a pre-determined threshold; or (b) at least one gene in the RB-E2F pathway with an alteration in its DNA (e.g., a mutation), an epigenetic alteration, an alteration in the level of expression of RNA (e.g., mRNA (e.g., pre-mRNA or mature mRNA)) or an alteration in the level of expression or activity of the encoded protein. Such a patient can be: treated with a platinum-based therapeutic agent (e.g., carboplatin or oxaliplatin) as a second agent; a patient whose cancer has developed resistance to a platinum-based therapeutic agent (e.g., carboplatin or oxaliplatin); or a patient undergoing treatment with a CDK4/6 inhibitor used alone or in combination with one or more of an aromatase inhibitor, a selective estrogen receptor modulator or a selective estrogen receptor degrader. The patient's cancer may have become resistant to the CDK4/6 inhibitor or at risk of becoming so. In the context of the uses described here (e.g., where the patient has been selected by virtue of having a level of BLC2-like 1 mRNA (e.g., pre-mRNA or mature mRNA) equal to or below the pre-determined threshold level), the cancer can be a breast cancer (e.g., a triple negative breast cancer (TNBC), an ovarian cancer, a lung cancer (e.g., non-small cell lung cancer), or a blood cancer (e.g., acute myeloid leukemia (AML)), any of which may be newly diagnosed (treatment naïve) or relapsed or refractory to a prior treatment.

The patient can be one who has undergone, is presently undergoing, or who will undergo (e.g., has been prescribed) treatment with a Bcl-2 inhibitor, such as venetoclax. In the context of this use, the patient can be selected by virtue of having one or more of: a) a level of CCNE1 gene copy number, mRNA (e.g., pre-mRNA or mature mRNA) or protein in the cancer equal to or above a pre-determined threshold; b) a level of RB1 gene copy number, mRNA or protein in the cancer equal to or below a pre-determined threshold, or an absence of an expressed wild-type RB1 gene; c) a level of CDK6 mRNA (e.g., pre-mRNA or mature mRNA) equal to or above a pre-determined threshold level; d) a level of CCND2 mRNA (e.g., pre-mRNA or mature mRNA) equal to or above a pre-determined threshold level; or e) a level of CDKN2A mRNA (e.g., pre-mRNA or mature mRNA) equal to or below a pre-determined threshold level. In specific embodiments, the patient is selected by virtue of having a level of CCNE1 gene copy number, mRNA or protein in the cancer equal to or above a pre-determined threshold; a level of RB1 gene copy number, mRNA or protein in the cancer equal to or below a pre-determined threshold; or an absence of an expressed wild-type RB1 gene. In the context of this use, the patient can be suffering from ovarian cancer, breast cancer, TNBC, or hormone receptor-positive breast cancer, and the patient may be one who has undergone, is presently undergoing, or will undergo treatment with a selective estrogen receptor modulator such as tamoxifen, a selective estrogen receptor degrader such as fulvestrant, and/or a PARP inhibitor, such as olaparib or niraparib.

In another aspect, the invention features the use of a CDK7 inhibitor described herein or a pharmaceutically acceptable salt thereof in treating a patient identified as described herein, with a combination therapy with an effective amount of a second agent in treating a patient who has cancer, wherein: (a) the cancer is TNBC, an estrogen receptor-positive (ER⁺) breast cancer, pancreatic cancer, or a squamous cell cancer of the head or neck; and the second agent is a CDK4/6 inhibitor; (b) the cancer is a breast cancer, or an ovarian cancer; and the second agent is a PARP inhibitor; (c) the cancer is AML; and the second agent is a FLT3 inhibitor; (d) the cancer is an ovarian cancer; and the second agent is a platinum-based anti-cancer agent; (e) the cancer is TNBC, AML, Ewing's sarcoma, or an osteosarcoma; and the second agent is a BET inhibitor; (f) the cancer is TNBC, AML, an ovarian cancer, or non-small cell lung cancer; and the second agent is a Bcl-2 inhibitor. In particular embodiments, the cancer is AML (e.g., of a monocytic subtype, e.g., an M4 or M5 subtype of AML) and the second agent is a Bcl-2 inhibitor, such as venetoclax; the cancer is an epithelial ovarian cancer, a fallopian tube cancer, a primary peritoneal cancer, a triple negative breast cancer or a Her2⁺/ER⁻/PR⁻ breast cancer and the second agent is a PARP inhibitor, such as olaparib or niraparib; the cancer is an ovarian cancer and the second agent is a platinum-based anti-cancer agent, such as carboplatin or oxaliplatin.

In one embodiment, the invention features methods of treating cancer, the methods including a step of administering an effective amount of a CDK7 inhibitor described herein or a pharmaceutically acceptable salt thereof to a patient (e.g., a human patient) identified as having a level of B-cell lymphoma-extra large (BCL-xL) mRNA (e.g., pre-mRNA or mature mRNA) in the cancer (e.g., in a biological sample obtained from the patient to be treated) that is equal to or below a pre-determined threshold (i.e., an “identified patient”). The methods can further include a step of determining the level of BCL-xL mRNA present in a sample of cancer cells from the patient, and this is generally true for the methods of treatment described herein; regardless of the biomarker analyzed or the type of cancer in question, a method of treatment can either be carried out on an identified patient without an explicit step of analyzing the biomarker or with an explicit step in which the biomarker is analyzed (e.g., by obtaining a biological sample from a patient). The human patient may have been diagnosed as having a cancer sensitive to a CDK7 inhibitor responsive to the determination, and the state of the BCL-xL biomarker can be determined in any of the additional ways described herein. The pre-determined threshold is a cutoff value or a prevalence cutoff A patient who is determined to have a cancer sensitive to a CDK7 inhibitor can additionally be administered a Bcl-2 inhibitor (e.g., venetoclax (available as Venclexta®)), and a patient selected as described here (through an analysis of the state of BCL-XL) can be suffering from a breast cancer, an ovarian cancer, a lung cancer, or a hematological cancer. More specifically, the patient can be suffering from TNBC, ovarian cancer, non-small cell lung cancer, or AML.

In one embodiment, the invention features methods of treating cancer (e.g., a breast cancer as described herein, including TNBC or a HR+ breast cancer), the methods including a step of administering an effective amount of a CDK7 inhibitor described herein or a pharmaceutically acceptable salt thereof to a patient (e.g., a human patient) identified by a mutation, copy number alteration (CNA), chromosomal translocation, or transcriptional upregulation of c-myc (as evidenced, e.g., by RNA (e.g., mRNA (e.g., pre-mRNA or mature mRNA)) levels equal to or above a pre-determined threshold level), a c-myc SE or SE strength above a pre-determined threshold, or an increase in the expression or activity of MYC (see Kalkat et al., Genes 8(6):151, 2017). C-myc encodes at least two phosphoproteins with apparent molecular weights of 62,000 and 66,000 (see Ramsay et al., Proc. Natl. Acad. Sci. (USA) 81(24):7742-7746, 1984), and it has been determined through H3K27Ac ChIP-seq (ChIP-sequencing) methods that there is a SE locus associated with the MYC gene at chr8:128628088-128778308 (Gencode v19 annotation of the human genome build hg19/GRCh37).

In the present methods, one can assess a CDK18 SE (identifying, for example, a CDK18 SE strength above a pre-determined threshold) or an FGFR1 SE (identifying, for example, an FGFR1 SE strength above a pre-determined threshold). In some embodiments, the method further includes a step of analyzing the SE (e.g., by determining its presence or absence and/or its strength) in a biological sample including cancer cells from the patient. The human patient may have been diagnosed as having a cancer sensitive to a CDK7 inhibitor responsive to the determination. A patient selected as described here (through an analysis of the state of MYC, CDK18, or FGFR1) can be suffering from a breast cancer (e.g., TNBC). A diagnosing step that identifies a patient can be based on the presence (or absence) or the strength of a MYC SE or a CDK18 SE. In these methods and others, the patient can be suffering from ovarian cancer and the diagnosis can be based on the presence, absence, or strength of a MYC, CDK18, or an FGFR1 SE.

In one embodiment, the invention features methods of diagnosing and treating a human patient having a cancer, the method including the steps of: (a) diagnosing whether the patient has a cancer sensitive to a CDK7 inhibitor based on the state of a biomarker selected from CDK7, CDK9, CDK18 and CDK19 (e.g., a level of CDK7, CDK9, CDK18, or CDK19 mRNA (e.g., pre-mRNA or mature mRNA)) previously determined by analyzing a sample of cancer cells from the patient; and (b) administering an effective amount of a CDK7 inhibitor described herein or a pharmaceutically acceptable salt thereof to a patient identified as having a cancer, wherein either: (i) the state of the CDK18 or CDK19 biomarker (e.g., the CDK18 or CDK19 mRNA (e.g., pre-mRNA or mature mRNA) level) is equal to or above a pre-determined threshold, or (ii) the state of the CDK7 or CDK9 biomarker (e.g., the CDK7 or CDK9 mRNA level (e.g., pre-mRNA or mature mRNA)) is equal to or below a pre-determined threshold (i.e., the “selected subject”). These methods can further include determining the state of a CDK biomarker selected from CDK7, CDK9, CDK18 and CDK19 in the cancer cells of the subject; determining by an active analytical step that may include obtaining a biological sample from a patient. The patient may have been diagnosed as having a cancer sensitive to a CDK7 inhibitor responsive to the determination. As in other embodiments, the CDK7 inhibitor can be a CDK7 inhibitor described herein or a pharmaceutically acceptable salt thereof. Where the biomarker is CDK7, CDK9, CDK18, or CDK19, the patient may have a lymphoma and the diagnosing/identifying step may more specifically be based on analysis of CDK7 (e.g., the level of CDK7 mRNA (e.g., pre-mRNA or mature mRNA)); the patient may have a TNBC, with the diagnosing/identifying step more specifically based on CDK9 (e.g., the level of CDK9 mRNA (e.g., pre-mRNA or mature mRNA)); the patient may have a TNBC, with the diagnosing step more specifically based on CDK18 (e.g., the level of CDK18 mRNA (e.g., pre-mRNA or mature mRNA)); the patient may have a TNBC or a SCLC, with the diagnosing step more specifically be based CDK19 (e.g., on the level of CDK19 mRNA (e.g., pre-mRNA or mature mRNA)).

With regard to combination therapies, a patient identified as described herein can be treated with a combination of a CDK7 inhibitor described herein or a pharmaceutically acceptable salt thereof and a second agent that can be, but is not limited to, a Bcl-2 inhibitor such as APG-1252, APG-2575, BP1002 (prexigebersen), the antisense oligonucleotide known as oblimersen (G3139), S55746/BCL201, or venetoclax (e.g., venetoclax tablets marketed as Venclexta®); a CDK9 inhibitor such as alvocidib/DSP-2033/flavopiridol, AT7519, AZD5576, BAY1251152, BAY1143572, CYC065, nanoflavopiridol, NVP2, seliciclib (CYC202), TG02, TP-1287, VS2-370 or voruciclib (formerly P1446A-05); a hormone receptor (e.g., estrogen receptor) degradation agent, such as fulvestrant (e.g., marketed as Faslodex® and others); a Flt3 (FMS-like tyrosine kinase 3) inhibitor such as CDX-301, CG′806, CT053PTSA, crenolanib (e.g., crenolanib besylate), ENMD-2076, FF-10101-01, FLYSYN, gilteritinib (ASP2215), HM43239, lestautinib, ponatinib (e.g., marketed as Iclusig®, previously AP24534), NMS-088, sorafenib (e.g., marketed as Nexavar®), sunitinib, pacritinib, pexidartinib/PLX3397, quizartinib, midostaurin (e.g., marketed as Rydapt®), SEL24, SKI-G-801, or SKLB1028; a PARP inhibitor such as olaparib (e.g., marketed as Lynparza®), rucaparib (e.g., marketed as Rubraca®), talazoparib (e.g., marketed as Talzenna®), veliparib (ABT-888), or niraparib (e.g., marketed as Zejula®); a BET inhibitor such as ABBV-075, BAY-299, BAY-1238097, BMS-986158, CPI-0610, CPI-203, FT-1101, GS-5829, GSK-2820151, GSK-525762, I-BET151, I-BET762, INCB054329, JQ1, MS436, OTX015, PFI-1, PLX51107, RVX2135, TEN-010, ZEN-3694, or a compound disclosed in U.S. application Ser. No. 12/810,564 (now U.S. Pat. No. 8,476,260), which is hereby incorporated herein by reference in its entirety; a platinum-based therapeutic agent such as cisplatin, oxaliplatin (e.g., marketed as Eloxatin®), nedaplatin, carboplatin (e.g., marketed as Paraplatin®), phenanthriplatin, picoplatin, satraplatin (JM216), or triplatin tetranitrate; a CDK4/6 inhibitor such as BPI-1178, G1T38, palbociclib (e.g., marketed as Ibrance®), ribociclib (e.g., marketed as Kisqali®), ON 123300, trilaciclib, or abemaciclib (e.g., marketed as Verzenio®); a MEK inhibitor such as trametinib (e.g., marketed as Mekinist®); or a phosphoinositide 3-kinase (PI3 kinase) inhibitor, optionally of Class I (e.g., Class IA) and/or optionally directed against a specific PI3K isoform. The PI3K inhibitor can be idelalisib (e.g., marketed as Zydelig®), copanlisib (e.g., marketed as Aligopa®), duvelisib (e.g., marketed as Copiktra®), or alpelisib (e.g., marketed as Piqray®). In other embodiments, the additional/second agent can be capecitabine (e.g., marketed as Xeloda®).

APG-1252 is a dual Bcl-2/Bcl-xL inhibitor that has shown promise in early clinical trials when patients having SCLC or another solid tumor were dosed between 10-400 mg (e.g., 160 mg) intravenously twice weekly for three weeks in a 28-day cycle (see Lakhani et al., J. Clin. Oncol. 36:15_suppl, 2594, and ClinicalTrials.gov identifier NCT03080311). APG-2575 is a Bcl-2 selective inhibitor that has shown promise in preclinical studies of FL and DLBCL in combination with ibrutinib (see Fang et al., AACR Annual Meeting 2019, Cancer Res. 79(13 Suppl):Abstract No. 2058) and has begun clinical trials as a single-agent treatment for patients with blood cancers; in a dose escalation study, patients are given 20 mg, once daily, by mouth, for four consecutive weeks as one cycle. Escalations to 50, 100, 200, 400, 600 and 800 mg are planned to identify the MTD (see ClinicalTrials.gov identifier NCT03537482). BP1002 is an uncharged P-ethoxy antisense oligodeoxynucleotide targeted against Bcl-2 mRNA that may have fewer adverse effects than other antisense analogs and has shown promise in inhibiting the growth of human lymphoma cell lines incubated with BP1002 for four days and of CJ cells (transformed FL cells) implanted into SCID mice (see Ashizawa et al., AACR Annual Meeting 2017, Cancer Res. 77(13 Suppl):Abstract No. 5091). BP1002 has also been administered in combination with cytarabine (LDAC) to patients having AML (see ClinicalTrials.gov identifier NCT04072458). S55746/BCL201 is an orally available, selective Bcl-2 inhibitor that, in mice, demonstrated anti-tumor efficacy in two blood cancer xenograft models (Casara et al., Oncotarget 9(28):20075-88, 2018). A phase I dose-escalation study was designed to administer film-coated tablets containing 50 or 100 mg of S55746, in doses up to 1500 mg, to patients with CLL or a B cell NHL including FL, MCL, DLBCL, SLL, MZL, and MM (see ClinicalTrials.gov identifier NCT02920697). Venetoclax tablets have been approved for treating adult patients with CLL or SLL and, in combination with azacytidine, or decitabine, or low-dose cytarabine, for treating newly-diagnosed AML in patients who are at least 75 years old or who have comorbidities that preclude the use of intensive induction chemotherapy. Dosing for CLL/SLL can follow the five-week ramp-up schedule and dosing for AML can follow the four-day ramp-up, both described in the product insert, together with other pertinent information (see also U.S. Pat. Nos. 8,546,399; 9,174,982; and 9,539,251, which are hereby incorporated by reference in their entireties). Alvocidib was studied in combination with cytarabine/mitoxantrone or cytarabine/daunorubicin in patients with AML, with the details of administration being available at ClinicalTrials.gov with the identifier NCT03563560 (see also Yeh et al., Oncotarget 6(5):2667-2679, 2015, Morales et al., Cell Cycle 15(4):519-527, 2016, and Zeidner et al., Haematologica 100(9):1172-1179, 2015). AT7519 has been administered in a dose escalation format to eligible patients having refractory solid tumors. While there was some evidence of clinical activity, the appearance of QTc prolongation precluded further development at the dose schedule described by Mahadevan et al. (J. Clin. Oncol. ASCO Abstract No. 3533; see also Santo et al., Oncogene 29:2325-2336, 2010, describing the preclinical activity of AT7519 in MM). AZD5576 induced apoptosis in breast and lung cancer cell lines at the nanomolar level (see Li et al., Bioorg. Med. Chem. Lett. 27(15):3231-3237, 2017) and has been examined alone and in combination with acalabrutinib for the treatment of NHL (see AACR 2017 Abstract No. 4295). BAY1251152 was the subject of a phase I clinical trial to characterize the MTD in patients with advanced blood cancers; the agent was infused weekly in 21-day cycles (see ClinicalTrials.gov identifier NCT02745743; see also Luecking et al., AACR 2017 Abstract No. 984). Voruciclib is a clinical stage oral CDK9 inhibitor that represses MCL-1 and sensitizes high-risk DLBCL to BCL2 inhibition. Dey et al. (Scientific Reports 7:18007, 2017) suggest that the combination of voruciclib and venetoclax is promising for a subset of high-risk DLBCL patients (see also ClinicalTrials.gov identifier NCT03547115). Fulvestrant has been approved for administration to postmenopausal women with advanced hormone receptor (HR)-positive, HER2-negative breast cancer, with HR-positive metastatic breast cancer whose disease progressed after treatment with other anti-estrogen therapies, and in combination with palbociclib (Ibrance®). Fulvestrant is administered by intramuscular injection at 500 or 250 mg (the lower dose being recommended for patients with moderate hepatic impairment) on days 1, 15, and 29, and once monthly thereafter (see the product insert for additional information; see also U.S. Pat. Nos. 6,744,122; 7,456,160; 8,329,680; and 8,466,139, each of which are hereby incorporated by reference herein in their entireties). Ponatinib has been administered in clinical trials to patients with CML or ALL (see ClinicalTrials.gov identifiers NCT0066092072, NCT012074401973, NCT02467270, NCT03709017, NCT02448095, NCT03678454, and NCT02398825) as well as solid tumors, such as biliary cancer and NSCLC (NCT02265341, NCT02272998, NCT01813734, NCT02265341, NCT02272998, NCT01813734, NCT02265341, NCT02272998, NCT01813734, NCT01935336, NCT03171389, and NCT03704688; see also the review article by Tan et al., Onco. Targets Ther. 12:635-645, 2019). Additional information regarding the dosing regimen can be found in the product insert; see also U.S. Pat. Nos. 8,114,874; 9,029,533; and 9,493,470, each of which is hereby incorporated by reference herein in its entirety. Sorafenib has been approved for the treatment of kidney and liver cancers, AML, and radioactive iodine resistant advanced thyroid cancer, and a clinical trial was initiated in patients with desmoid-type fibromatosis (see ClinicalTrials.gov identifier NCT02066181). Information regarding dosage can be found in the product insert, which advises administration of two, 400 mg tablets twice daily; see also U.S. Pat. Nos. 7,235,576; 7,351,834; 7,897,623; 8,124,630; 8,618,141; 8,841,330; 8,877,933; and 9,737,488, each of which is hereby incorporated by reference herein in its entirety. Midostaurin has been administered to patients having AML, MDS, or systemic mastocytosis, and has been found to significantly prolong survival of FLT3-mutated AML patients when combined with conventional induction and consolidation therapies (see Stone et al., ASH 57th Annual Meeting, 2015 and Gallogly et al., Ther. Adv. Hematol. 8(9):245-251, 2017; clin see also the product insert, ClinicalTrials.gov identifier NCT03512197, and U.S. Pat. Nos. 7,973,031; 8,222,244; and 8,575,146, each of which is hereby incorporated by reference herein in its entirety. The information provided here and publicly available can be used to practice the methods and uses of the invention. In case of doubt, the invention encompasses combination therapies that require a compound of the invention or a pharmaceutically acceptable salt thereof and any one or more additional/second agents, which may be administered at or below a dosage currently approved for single use (e.g., as described above), to a patient as described herein.

Where the combination therapy employs a CDK7 inhibitor as described herein and: a CDK4/6 inhibitor, the patient can have a breast cancer (e.g., TNBC or an ER+ breast cancer), pancreatic cancer, lung cancer (e.g., SCLC or NSCLC), or squamous cell cancer of the head and neck; a CDK9 inhibitor, the patient can have a breast cancer and, more specifically, a Her2⁺/ER⁻/PR⁻ breast cancer; a Flt3 inhibitor (e.g., midostaurin), the patient can have a hematological cancer (e.g., AML); a BET inhibitor, the patient can have a hematological cancer (e.g., AML), a breast cancer (e.g., TNBC), an osteosarcoma or Ewing's Sarcoma; a Bcl-2 inhibitor (e.g., venetoclax), the patient can have a breast cancer (e.g., TNBC), an ovarian cancer, a lung cancer (e.g., NSCLC) or a hematological cancer (e.g., AML (e.g., newly diagnosed AML), chronic lymphocytic leukemia (CLL) or small lymphocytic lymphoma (SLL), with or without 17p deletion); or a PARP inhibitor (e.g., niraparib or olaparib), the patient can have a breast cancer (e.g., TNBC or Her2⁺/ER⁻/PR⁻ breast cancer), an ovarian cancer (e.g., an epithelial ovarian cancer), a fallopian tube cancer, or a primary peritoneal cancer. Where a patient is treated with a CDK7 inhibitor as described herein and a Bcl-2 inhibitor (e.g., venetoclax), the patient can be treated with a third agent as well, selected from azacitidine, decitabine, and low-dose cytarabine.

The invention provides pharmaceutical kits for treating cancer comprising a CDK7 inhibitor described herein or a pharmaceutically acceptable salt thereof and, optionally, a second therapeutic agent selected from: (a) a Bcl-2 inhibitor, (b) a CDK9 inhibitor, (c) a Flt3 inhibitor, (d) a PARP inhibitor, (e) a BET inhibitor, and (f) a CDK4/6 inhibitor, any of which may be selected from those disclosed herein and administered as described herein or as directed by the manufacturer. The kit can include optional instructions for: (a) reconstituting (if necessary) a CDK7 inhibitor described herein or a pharmaceutically acceptable salt thereof and/or the second therapeutic agent; (b) administering each of t a CDK7 inhibitor described herein or a pharmaceutically acceptable salt thereof and/or the second therapeutic agent; and/or (c) a list of specific cancers for which the kit is useful or diagnostic methods by which they may be determined. The kit can also include any type of paraphernalia useful in administering the active agent(s) contained therein (e.g., tubing, syringes, needles, sterile dressings, tape, and the like).

The invention provides methods of treating a human patient having a cancer, the method comprising: administering to a patient identified as having in at least one of the genes involved in the RB-E2F pathway: (1) an alteration in the DNA (e.g. gene copy number, mutation, methylation); (2) an epigenetic alteration (e.g. histone methylation, histone acetylation); or (3) an alteration in the level of expression of RNA (e.g., mRNA (e.g., pre-mRNA or mature mRNA)) or protein, an effective amount of a CDK7 inhibitor described herein or a pharmaceutically acceptable salt thereof. The patient is one identified (i.e., selected) as having an alteration in the level of mRNA (e.g., pre-mRNA or mature mRNA) expressed from at least one gene involved in the RB-E2F pathway. In this aspect, the patient is determined to have either a level of RNA (e.g., mRNA (e.g., pre-mRNA or mature mRNA)) of the at least one gene involved the Rb-E2F pathway equal to or above a pre-determined threshold or a level of RNA (e.g., mRNA (e.g., pre-mRNA or mature mRNA)) of the at least one gene involved the RB-E2F pathway equal to or below a pre-determined threshold, prior to administering to the patient an effective amount of a CDK7 inhibitor described herein or a pharmaceutically acceptable salt thereof.

It will be readily apparent to one of ordinary skill in the art that for those genes in the RB-E2F pathway that are activated or overexpressed in cancer, one would select those patients having (1) an alteration in the DNA encoding such gene that resulted in increased expression (e.g. elevated gene copy number, mutation that led to increased activity, change in methylation that led to increased expression); (2) an epigenetic alteration associated with that gene that resulted in increased expression (e.g. histone methylation or histone acetylation pattern that led to increased expression); or (3) an increase in the level of expression of mRNA (e.g., pre-mRNA or mature mRNA) or protein encoded by that gene. For those genes in the RB-E2F pathway that are inactivated or under-expressed in cancer, one would select from those patients having (1) an alteration in the DNA encoding that gene that resulted in decreased expression or activity (e.g. reduced gene copy number, mutation that led to decreased activity or inactivity, change in methylation that led to decreased expression); (2) an epigenetic alteration associated with that gene that resulted in decreased expression (e.g. histone methylation or histone acetylation pattern that led to decreased expression); or (3) an decrease in the level of expression of mRNA (e.g., pre-mRNA or mature mRNA) or protein encoded by that gene.

In some aspects relating to using RB-E2F pathway genes as biomarkers, the invention provides a method of treating a human patient having a cancer, which comprises administering to a patient identified as having either (a) a level of CCNE1 mRNA (e.g., pre-mRNA or mature mRNA) or protein in the cancer equal to or above a pre-determined threshold; and/or (b) a level of RB1 mRNA (e.g., pre-mRNA or mature mRNA) or protein in the cancer equal to or below a pre-determined threshold, an effective amount of a CDK7 inhibitor. In some embodiments of these methods, one can also determine a level of RB1 and/or CCNE1 mRNA (e.g., pre-mRNA or mature mRNA) or protein present in a sample of cancer cells from the patient. In some embodiments, the human patient is diagnosed as having a cancer sensitive to a CDK7 inhibitor responsive to the determination; the human patient is suffering from ovarian cancer; and/or the human patient is suffering from a breast cancer. In some embodiments, the human patient is suffering from a triple negative breast cancer (TNBC) or a hormone-receptor positive (HR⁺ (e.g., HR+/HER2−) breast cancer. In any of these embodiments, the CDK7 inhibitor is a CDK7 inhibitor described herein or a pharmaceutically acceptable salt thereof, which inhibitor or its salt is optionally co-administered with a PARP inhibitor or a SERM or a SERD such as tamoxifen or fulvestrant. In some of these embodiments, the cancer is refractory to palbociclib.

The invention provides a method of treating a cancer in a human patient by administering to the patient a combination of a CDK7 inhibitor and a platinum-based standard of care (SOC) anti-cancer agent for such cancer or a taxane. The cancer can be an ovarian cancer; the SOC anti-cancer agent can be a platinum-based anti-cancer agent (e.g., carboplatin, cisplatin, or oxaliplatin); and a CDK7 inhibitor described herein or a pharmaceutically acceptable salt thereof. In some embodiments, the human patient is, has been determined to be, or has become resistant (after some initial responsiveness) to the platinum-based anti-cancer agent when administered as either a monotherapy or in combination with an anti-cancer agent other than a CDK7 inhibitor. In some aspects of this embodiment, the human patient is determined to have become resistant to the platinum-based anti-cancer agent when administered as a monotherapy or in combination with an anti-cancer agent other than a CDK7 inhibitor after some initial efficacy of that prior treatment. In some aspects of this embodiment, the SOC anti-cancer agent is a taxane (e.g., paclitaxel).

The invention provides a method of treating HR⁺ breast cancer in a human patient selected on the basis of being resistant to treatment with a CDK4/6 inhibitor comprising the step of administering to the patient a CDK7 inhibitor described herein or a pharmaceutically acceptable salt thereof. In some embodiments, prior to administration of a CDK7 inhibitor described herein or a pharmaceutically acceptable salt thereof the patient is, has been determined to be, or has become resistant (after some initial responsiveness) to a prior treatment with a CDK4/6 inhibitor alone or in combination with another SOC agent for breast cancer other than a CDK7 inhibitor, such as an aromatase inhibitor (e.g., letrozole, anastrozole) or a SERM or SERD such as tamoxifen or fulvestrant. In other words, the identified patient is selected for treatment with a CDK7 inhibitor described herein or a pharmaceutically acceptable salt thereof on the basis of being resistant to prior treatment with a CDK4/6 inhibitor alone or in combination with another SOC agent for breast cancer other than a CDK7 inhibitor. In some embodiments, a CDK7 inhibitor described herein or a pharmaceutically acceptable salt thereof is co-administered with another SOC agent, such as an aromatase inhibitor (e.g. anastrozole, exemestane, or letrozole) or a SERM or SERD such as tamoxifen or fulvestrant, or a second line treatment after failure on an aromatase inhibitor or fulvestrant. In some embodiments, prior to administration of a CDK7 inhibitor described herein or a pharmaceutically acceptable salt thereof the patient is, has been determined to be, or has become resistant (after some initial responsiveness) to treatment with a CDK4/6 inhibitor alone or in combination with another SOC agent for breast cancer other than a CDK7 inhibitor, such as an aromatase inhibitor (e.g., anastrozole, exemestane, or letrozole), or a SERM or SERD such as tamoxifen or fulvestrant; and a CDK7 inhibitor described herein or a pharmaceutically acceptable salt thereof is co-administered with a SOC agent for breast cancer (e.g., a second line treatment after failure of an aromatase inhibitor or a SERM or SERD such as tamoxifen or fulvestrant).

The invention provides a method of diagnosing and treating a human patient having a cancer, the method comprising: (a) diagnosing whether the patient has a cancer sensitive to a CDK7 inhibitor based on the level of FGFR1, CDK6, CCND2, or CDKNA2, or the absence of a wild-type RB1 gene previously determined in a sample of cancer cells from the patient; and (b) administering an effective amount of a CDK7 inhibitor to a patient identified as having a cancer wherein: (a) the level of FGFR1, CDK6, or CCND2A mRNA (e.g., pre-mRNA or mature mRNA) is equal to or above a pre-determined threshold level; (b) the level of CDKN2A mRNA (e.g., pre-mRNA or mature mRNA) is equal to or below a pre-determined threshold level; or (c) the patient lacks the presence of a wild-type RB1 gene. In some aspects of these embodiments, the compound is a CDK7 inhibitor described herein or a pharmaceutically acceptable salt thereof. In some aspects of these embodiments, the cancer is ovarian cancer.

In a related embodiment, the invention provides methods of treating cancer in a human patient selected on the basis of the cancer having one or more of: (a) a level of FGFR1 mRNA (e.g., pre-mRNA or mature mRNA) equal to or above a pre-determined threshold level; (b) a level of CDK6 mRNA (e.g., pre-mRNA or mature mRNA) equal to or above a pre-determined threshold level; (c) a level of CCND2 mRNA (e.g., pre-mRNA or mature mRNA) equal to or above a pre-determined threshold level; (d) a level of CDKN2A mRNA (e.g., pre-mRNA or mature mRNA) equal to or below a pre-determined threshold level; or (e) an absence of a wild-type RB1 gene, wherein the selected patient is administered a CDK7 inhibitor described herein or a pharmaceutically acceptable salt thereof. In some embodiments, the cancer is ovarian cancer.

An enhancer or SE can be identified by various methods known in the art (see Hinsz et al., Cell, 155:934-947, 2013; McKeown et al., Cancer Discov., 7(10):1136-53, 2017; and PCT/US2013/066957, each of which are hereby incorporated herein by reference in their entireties). Identifying a SE can be achieved by obtaining a biological sample from a patient (e.g., from a biopsy or other source, as described herein). The important metrics for enhancer measurement occur in two dimensions: along the length of the DNA over which genomic markers (e.g., H3K27Ac) are contiguously detected and the compiled incidence of genomic marker at each base pair along that span of DNA, the compiled incidence constituting the magnitude. The measurement of the area under the curve (“AUC”) resulting from integration of length and magnitude analyses determines the strength of the enhancer. The strength of the BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table for those upregulated) SEs relative to an appropriate reference can be used to diagnose (stratify) a patient and thereby determine whether a patient is likely to respond well to a CDK7 inhibitor described herein or a pharmaceutically acceptable salt thereof. It will be readily apparent to one of ordinary skill in the art, particularly in view of the instant specification, that if the length of DNA over which the genomic markers is detected is the same for each of BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table for those upregulated) and the reference/control, then the ratio of the magnitude of the BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table for those upregulated) SE relative to the control will be equivalent to the strength and may also be used to determine whether a patient will be responsive to a CDK7 inhibitor described herein or a pharmaceutically acceptable salt thereof. The strength of the BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table for those upregulated) SE in a cell can be normalized before comparing it to other samples. Normalization is achieved by comparison to a region in the same cell known to comprise a ubiquitous SE or enhancer that is present at similar levels in all cells. One example of such a ubiquitous super-enhancer region is the MALAT1 super-enhancer locus (chr11:65263724-65266724) (genome build hg19).

It has been determined through H3K27Ac ChIP-seq (ChIP-sequencing) methods that there is a SE locus associated with a SE locus associated with the CDK18 gene at chr1:205399084-205515396; a SE locus associated with the CDK19 gene at chr6:110803523-110896277; a SE locus associated with the CCNE1 gene at chr19:30418503-30441450; and a SE locus associated with the FGFR1 gene at chr8:38233326-38595483. All loci are based on the Gencode v19 annotation of the human genome build hg19/GRCh37.

ChIP-seq is used to analyze protein interactions with DNA by combining chromatin immunoprecipitation (ChIP) with massively parallel DNA sequencing to identify the binding sites of DNA-associated proteins. It can be used to map global binding sites precisely for any protein of interest. Previously, ChIP-on-chip was the most common technique utilized to study these protein-DNA relations. Successful ChIP-seq is dependent on many factors including sonication strength and method, buffer compositions, antibody quality, and cell number (see, e.g., Furey, Nature Reviews Genetics 13:840-852, 2012); Metzker, Nature Reviews Genetics 11:31-46, 2010; and Park, Nature Reviews Genetics 10:669-680, 2009). Genomic markers other than H3K27Ac that can be used to identify SEs using ChIP-seq include P300, CBP, BRD2, BRD3, BRD4, components of the mediator complex (Loven et al., Cell, 153(2):320-334, 2013), histone 3 lysine 4 monomethylated (H3K4me1), and other tissue-specific enhancer tied transcription factors (Smith and Shilatifard, Nature Struct. Mol. Biol., 21(3):210-219, 2014; and Pott and Lieb, Nature Genetics, 47(1):8-12, 2015). Quantification of enhancer strength and identification of SEs can be determined using SE scores (McKeown et al., Cancer Discov. 7(10):1136-1153, 2017; DOI: 10.1158/2159-8290.CD-17-0399).

In some instances, H3K27Ac or other marker ChIP-seq data SE maps of the entire genome of a cell line or a patient sample already exist. One would then simply determine whether the strength, ordinal rank, or prevalence rank of the enhancer or SE in such maps at the chr8:128628088-128778308 (genome build hg19) locus was equal to or above the pre-determined threshold level. In some embodiments, one would simply determine whether the strength, or ordinal rank of the enhancer or super-enhancer in such maps at the chr1:205399084-205515396 (genome build hg19) locus was equal to or above the pre-determined threshold level.

It should be understood that the specific chromosomal location of BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) and MALAT1 may differ for different genome builds and/or for different cell types. The same is true for BCL2-like 1, CDK7, CDK9, CDKN2A, and RB (also known as RB1 or another E2F pathway member that is underexpressed in cancer (see the Table herein). However, one of ordinary skill, particularly in view of the present teaching, can determine such different locations by locating in such other genome builds specific sequences corresponding to the loci in genome build hg 19.

Other methods that can be used to identify SEs in the context of the present methods include chromatin immunoprecipitation (Delmore et al., Cell, 146(6):904-917, 2011), chip array (ChIP-chip), and chromatin immunoprecipitation followed by qPCR (ChIP-qPCR) using the same immunoprecipitated genomic markers and oligonucleotide sequences that hybridize to the chr8:128628088-128778308 (genome build hg19) MYC locus or chr1:205399084-205515396 (genome build hg19) CDK18 locus (for example). In the case of ChIP-chip, the signal is typically detected by intensity fluorescence resulting from hybridization of a probe and input assay sample as with other array-based technologies. For ChIP-qPCR, a dye that becomes fluorescent after intercalating the double stranded DNA generated in the PCR reaction is used to measure amplification of the template.

In some embodiments, determination of whether a cell has a BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) SE strength equal to or above a requisite threshold level is achieved by comparing BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength in a test cell to the corresponding BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) strength in a population of cell samples, wherein each of the cell samples is obtained from a different source (e.g., a different patient, a different cell line, a different xenograft) reflecting the same disease to be treated. In some embodiments, only primary tumor cell samples from patients are used to determine the threshold level. In some aspects of these embodiments, at least some of the samples in the population will have been tested for responsiveness to a specific CDK7 inhibitor (e.g., a CDK7 inhibitor described herein or a pharmaceutically acceptable salt thereof) to establish: (a) the lowest M BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength of a sample in the population that responds to that specific compound (“lowest responder”); and, optionally, (b) the highest BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength of a sample in the population that does not respond to that specific compound (“highest non-responder”). In these embodiments, a cutoff of BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength above which a test cell would be considered responsive to that specific compound is set: i) equal to or up to 5% above the BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength in the lowest responder in the population; or ii) equal to or up to 5% above the BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength in the highest non-responder in the population; or iii) a value in between the BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength of the lowest responder and the highest non-responder in the population.

In the above embodiments, not all of the samples in a population necessarily are to be tested for responsiveness to a specific CDK7 inhibitor (e.g., a CDK7 inhibitor described herein or a pharmaceutically acceptable salt thereof), but all samples are measured for BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength. In some embodiments, the samples are rank ordered based on M BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength. The choice of which of the three methods set forth above to use to establish the cutoff will depend upon the difference in BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength between the lowest responder and the highest non-responder in the population and whether the goal is to minimize the number of false positives or to minimize the chance of missing a potentially responsive sample or patient. When the difference between the lowest responder and highest non-responder is large (e.g., when there are many samples not tested for responsiveness that fall between the lowest responder and the highest non-responder in a rank ordering of BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength), the cutoff is typically set equal to or is up to 5% above the BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength in the lowest responder in the population. This cutoff maximizes the number of potential responders. When this difference is small (e.g., when there are few or no samples untested for responsiveness that fall between the lowest responder and the highest non-responder in a rank ordering of BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength), the cutoff is typically set to a value in between the BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength of the lowest responder and the highest non-responder. This cutoff minimizes the number of false positives. When the highest non-responder has a BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength that is greater than the lowest responder, the cutoff is typically set to a value equal to or up to 5% above the BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) enhancer strength in the highest non-responder in the population. This method also minimizes the number of false positives.

In some embodiments, the methods discussed above can be employed to simply determine if a diseased cell (e.g., a cancer cell) from a patient has a SE associated with a biomarker as described herein (e.g., BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) or a protein encoded thereby). The presence of the SE indicates that the patient is likely to respond well to a CDK7 inhibitor described herein or a pharmaceutically acceptable salt thereof. The cell is determined to have a SE associated with the biomarker (e.g., BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) or a protein encoded thereby) when the enhancer has a strength that is equal to or above the enhancer associated with MALAT-1. In alternate embodiments, the cell is determined to have a SE associated with BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) when the BRAF-, MYC-, CDK1-, CDK2-, CDK4-, CDK6-, CDK17-, CDK18-, CDK19-, CCNA1-, CCNB1-, CCNE1-, ESR-1-, FGFR1-, PIC3CA-, or certain genes encoding an E2F pathway member- (see the Table herein) associated enhancer has a strength that is at least 10-fold greater than the median strength of all of the enhancers in the cell. In other embodiments, the cell is determined to have a SE associated with an aforementioned gene when the gene-associated enhancer has a strength that is above the point where the slope of the tangent is 1 in a rank-ordered graph of strength of the enhancers in the cell.

In embodiments involving CDK18, the cutoff value for enhancer strength can be converted to a prevalence cutoff, which can then be applied to CDK18 mRNA (e.g., pre-mRNA or mature mRNA) levels to determine an mRNA cutoff value in a given mRNA assay.

In some embodiments, a feature of a genetic biomarker described herein (e.g., mRNA (e.g., pre-mRNA or mature mRNA) levels) are used to determine a patient's sensitivity to a CDK7 inhibitor described herein or a pharmaceutically acceptable salt thereof.

In some embodiments, gene of interest/biomarker mRNA levels in a patient (as assessed, e.g., in a biological sample obtained from the patient) are compared, using the same assay, to the same gene of interest/biomarker mRNA levels in a population of patients having the same disease or condition (or a disease or condition that is similar enough to allow for effective comparison) to identify likely responders to a CDK7 inhibitor described herein or a pharmaceutically acceptable salt thereof. Analogous comparisons can be made when another feature of the biomarker is selected for analysis (e.g., its copy number, chromosomal location, primary RNA transcript level or mRNA (e.g., pre-mRNA or mature mRNA) level, or expressed protein level). In embodiments where a biomarker (e.g., CDK18, CDK19, and CCNE1) correlates with (e.g., is one whose expression (e.g., mRNA (e.g., pre-mRNA or mature mRNA) expression) correlates with) responsiveness to a compound of the invention, at least some of the samples in the population will have been tested for responsiveness to the inhibitor (e.g., a CDK7 inhibitor described herein or a pharmaceutically acceptable salt thereof) to establish: (a) the lowest level (e.g., mRNA level) in a sample in the population that responds to that specific compound (“lowest mRNA responder”); and, optionally, (b) the highest level (e.g., highest mRNA level) in a sample in the population that does not respond to that specific compound (“highest mRNA non-responder”). In these embodiments, a cutoff of biomarker mRNA level above which a test cell would be considered responsive to that specific compound is set: i) equal to or up to 5% above the level (e.g., the mRNA level) in the lowest mRNA responder in the population; or ii) equal to or up to 5% above the level (e.g., the mRNA level) in the highest mRNA non-responder in the population; or iii) a value in between the level (e.g., mRNA level) of the lowest responder (e.g., lowest mRNA responder) and the highest responder (e.g., highest mRNA) non-responder in the population.

In embodiments where mRNA (e.g., pre-mRNA or mature mRNA) levels positively correlate with sensitivity to a CDK7 inhibitor described herein or a pharmaceutically acceptable salt thereof, not all of the samples in a population need to be tested for responsiveness to the compound (or the salt), but all samples are measured for the gene of interest mRNA levels. In some embodiments, the samples are rank ordered based on gene of interest mRNA levels. The choice of which of the three methods set forth above to use to establish the cutoff will depend upon the difference in gene of interest mRNA levels between the lowest mRNA responder and the highest mRNA non-responder in the population and whether the cutoff is designed to minimize false positives or maximize the potential number of responders. When this difference is large (e.g., when there are many samples not tested for responsiveness that fall between the lowest mRNA responder and the highest mRNA non-responder in a rank ordering of mRNA levels), the cutoff is typically set equal to or up to 5% above the mRNA level in the lowest mRNA responder. When this difference is small (e.g., when there are few or no samples untested for responsiveness that fall between the lowest mRNA responder and the highest mRNA non-responder in a rank ordering of mRNA levels), the cutoff is typically set to a value in between the mRNA levels of the lowest mRNA responder and the highest mRNA non-responder. When the highest mRNA non-responder has a mRNA level that is greater than the lowest mRNA responder, the cutoff is typically set to a value equal to or up to 5% above the mRNA levels in the highest mRNA non-responder in the population.

In embodiments where a gene of interest/biomarker is one whose mRNA (e.g., pre-mRNA or mature mRNA) expression inversely correlates with responsiveness to a CDK7 inhibitor described herein or a pharmaceutically acceptable salt thereof (i.e., BCL-xL, CDK7, CDK9, or an RB1 family member), at least some of the samples in the population will have been tested for responsiveness to the compound in order to establish: (a) the highest mRNA level of a sample in the population that responds to that specific compound (“highest mRNA responder”); and, optionally, (b) the lowest mRNA level of a sample in the population that does not respond to that specific compound (“lowest mRNA non-responder”). In these embodiments, a cutoff of mRNA level above which a test cell would be considered responsive to that specific compound is set: i) equal to or up to 5% below the mRNA level in the highest mRNA responder in the population; or ii) equal to or up to 5% below the mRNA level in the lowest mRNA non-responder in the population; or iii) a value in between the mRNA level of the lowest mRNA non-responder and the highest mRNA responder and in the population.

In embodiments where mRNA (e.g., pre-mRNA or mature mRNA) levels inversely correlate with sensitivity to a compound of the invention, not all of the samples in a population need to be tested for responsiveness to the compound, but all samples are measured for the gene of interest mRNA levels. In some embodiments, the samples are rank ordered based on gene of interest mRNA levels. The choice of which of the three methods set forth above to use to establish the cutoff will depend upon the difference in gene of interest mRNA levels between the highest mRNA responder and the lowest mRNA non-responder in the population and whether the cutoff is designed to minimize false positives or maximize the potential number of responders. When this difference is large (e.g., when there are many samples not tested for responsiveness that fall between the highest mRNA responder and the lowest mRNA non-responder in a rank ordering of mRNA levels), the cutoff is typically set equal to or up to 5% below the mRNA level in the highest mRNA responder. When this difference is small (e.g., when there are few or no samples untested for responsiveness that fall between the highest mRNA responder and the lowest mRNA non-responder in a rank ordering of mRNA levels), the cutoff is typically set to a value in between the mRNA levels of the highest mRNA responder and the lowest mRNA non-responder. When the highest mRNA responder has a mRNA level that is lower than the lowest mRNA responder, the cutoff is typically set to a value equal to or up to 5% below the mRNA levels in the lowest mRNA non-responder in the population.

In embodiments involving CDK18, the cutoff for CDK18 mRNA (e.g., pre-mRNA or mature mRNA) levels may be determined using the prevalence cutoff established based on CDK18 enhancer strength, as described above. In some aspects of these embodiments, a population is measured for mRNA levels and the prior determined prevalence cutoff is applied to that population to determine an mRNA cutoff level. In some aspects of these embodiments a rank-order standard curve of CDK18 mRNA levels in a population is created, and the pre-determined prevalence cutoff is applied to that standard curve to determine the CDK18 mRNA cutoff level.

In some embodiments where a test cell or sample is compared to a population, the cutoff mRNA (e.g., pre-mRNA or mature mRNA) level value(s) obtained for the population is converted to a prevalence rank and the mRNA level cutoff is expressed as a percent of the population having the cutoff value or higher, e.g., a prevalence cutoff.

Without being bound by theory, the Applicant believes the prevalence rank of a test sample and the prevalence cutoff in a population will be similar regardless of the methodology used to determine mRNA levels.

A patient can be identified as likely to respond well to a CDK7 inhibitor described herein or a pharmaceutically acceptable salt thereof if the state of BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein) as determined by, e.g., RNA (e.g., mRNA (e.g., pre-mRNA or mature mRNA) levels) in a biological sample from the patient) corresponds to (e.g., is equal to or greater than) a prevalence rank in a population of about 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 43%, 42%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20% as determined by the state of BRAF, MYC, CDK1, CDK2, CDK4, CDK6, CDK17, CDK18, CDK19, CCNA1, CCNB1, CCNE1, ESR-1, FGFR1, PIC3CA, or certain genes encoding an E2F pathway member (see the Table herein), respectively, determined by assessing the same parameter (e.g., mRNA level(s)) in the population. A patient can be identified as likely to respond well to a CDK7 inhibitor described herein or a pharmaceutically acceptable salt thereof if the state of BCL2-like 1, CDK7, CDK9, CDKN2A, and RB (as determined by, e.g., RNA (e.g., mRNA (e.g., pre-mRNA or mature mRNA)) levels or corresponding protein levels in a biological sample from the patient) is below a prevalence rank in a population of about 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%4, 43%, 42%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20% as determined by the state of BCL2-like 1, CDK7, CDK9, CDKN2A, and RB, respectively, determined by assessing the same parameter (e.g., mRNA level(s)) in the population. In some embodiments, the cutoff value or threshold is established based on the biomarker (e.g., mRNA) prevalence value.

In some embodiments, a population may be divided into three groups: responders, partial responders and non-responders, and two cutoff values (or thresholds) or prevalence cutoffs are set or determined. The partial responder group may include responders and non-responders as well as those patients whose response to a CDK7 inhibitor described herein or a pharmaceutically acceptable salt thereof was not as high as the responder group. This type of stratification may be particularly useful when, in a population, the highest non-responder has an value (e.g., an mRNA (e.g., pre-mRNA or mature mRNA) level) that is greater than that of the lowest responder (assessed for the same parameter (e.g., mRNA levels)). In this scenario, for CDK18 or CDK19, the cutoff level or prevalence cutoff between responders and partial responders is set equal to or up to 5% above the CDK18 or CDK19 mRNA level of the highest CDK18 or CDK19 mRNA non-responder; and the cutoff level or prevalence cutoff between partial responders and non-responders is set equal to or up to 5% below the CDK18 or CDK19 mRNA level of the lowest CDK18 or CDK19 mRNA responder. For BCL-xL, CDK7 or CDK9, this type of stratification may be useful when the highest mRNA responder has a mRNA level that is lower than that of the lowest mRNA non-responder. In this scenario, for BCL-xL, CDK7 or CDK9, the cutoff level or prevalence cutoff between responders and partial responders is set equal to or up to 5% below the mRNA level of the lowest mRNA non-responder; and the cutoff level or prevalence cutoff between partial responders and non-responders is set equal to or up to 5% above the mRNA level of the highest mRNA responder. The determination of whether partial responders should be administered a CDK7 inhibitor described herein or a pharmaceutically acceptable salt thereof will depend upon the judgment of the treating physician and/or approval by a regulatory agency.

Methods that can be used to quantify specific primary RNA transcripts in a biological sample are known in the art and include, but are not limited to, fluorescent hybridization such as utilized in services and products provided by NanoString Technologies, array based technology (Affymetrix), reverse transcriptase qPCR as with SYBR® Green (Life Technologies) or TaqMan® technology (Life Technologies), RNA sequencing (e.g., RNA-seq), RNA hybridization and signal amplification as utilized with RNAscope® (Advanced Cell Diagnostics), or Northern blot. In some cases, mRNA expression values for various genes in various cell types are publicly available (see, e.g., broadinstitute.org/ccle; and Barretina et al., Nature, 483:603-607, 2012).

In some embodiments, the state of a biomarker (as assessed, for example, by the level of RNA transcripts) in both the test biological sample and the reference standard or all members of a population is normalized before comparison. Normalization involves adjusting the determined level of a primary RNA transcript by comparison to either another RNA transcript that is native to and present at equivalent levels in both of the cells (e.g., GADPH mRNA, 18S RNA), or to a fixed level of exogenous RNA that is “spiked” into samples of each of the cells prior to super-enhancer strength determination (Lovén et al., Cell, 151(3):476-82, 2012; Kanno et al., BMC Genomics 7:64, 2006; Van de Peppel et al., EMBO Rep., 4:387-93, 2003).

A patient (e.g., a human) suffering from a cancer described herein and identified as described herein based on biomarker status may have been determined to be resistant (or to be acquiring resistance after some initial efficacy) to a therapeutic agent that was administered prior to a CDK7 inhibitor described herein or a pharmaceutically acceptable salt thereof and/or resistant to a previously administered chemotherapeutic agent (e.g., a Bcl-2 inhibitor such as venetoclax, a BET inhibitor, a CDK4/6 inhibitor such as palbociclib or ribociclib, a CDK9 inhibitor such as alvocidib, a FLT3 inhibitor, a MEK inhibitor such a trametinib, a PARP inhibitor, such as olaparib or niraparib, a PI3K inhibitor, such as alpelisib or capecitabine, a platinum-based therapeutic agent such as cisplatin, oxaliplatin, nedaplatin, carboplatin, phenanthriplatin, picoplatin, satraplatin (JM216), or triplatin tetranitrate, a SERM, such as tamoxifen faloxifene, or toremifene, or a steroid receptor degrading agent (e.g., a SERD, such as fulvestrant). Combination therapies including one or more of these agents (wherein administration is made to a patient selected as described herein or otherwise) are also within the scope of the invention and are discussed further herein. For example, in one embodiment, the methods encompass the use of or administration of a CDK7 inhibitor described herein or a pharmaceutically acceptable salt thereof in combination with a SERD, such as fulvestrant, to treat a cancer (e.g., a breast cancer (e.g., an ER+ breast cancer (e.g., an HR+/HER2− cancer))) resistant to treatment with a CDK4/6 inhibitor such as palbociclib or ribociclib.

In some embodiments, the prior therapeutic agent may be a platinum-based anti-cancer agent administered as a monotherapy or in combination with a SOC agent. Most cancer patients eventually develop resistance to platinum-based therapies by one or more of the following mechanisms: (i) molecular alterations in cell membrane transport proteins decrease uptake of the platinum agent; (ii) molecular alterations in apoptotic signaling pathways that prevent a cell from inducing cell death; (iii) molecular alterations of certain genes (e.g. BRCA1/2, CHEK1, CHEK2, RAD51) that restore the ability of the cell to repair platinum agent-induced DNA damage. K. N. Yamamoto et al., PloS ONE 9(8):e105724, 2014. The term “molecular alterations” includes increased or decreased mRNA (e.g., pre-mRNA or mature mRNA) expression from the genes involved in these functions; increased or decreased expression of protein from such genes; and mutations in the mRNA/proteins expressed from those genes.

Resistance is typically determined by disease progression (e.g., an increase in tumor size and/or numbers) during treatment or a decrease in the rate of shrinkage of a tumor. In some instances, a patient will be considered to have become resistant to a platinum-based agent when the patient's cancer responds or stabilizes while on treatment, but which progresses within 1-6 months following treatment with the agent. Resistance can occur after any number of treatments with platinum agents. In some instances, disease progression occurs during, or within 1 month of completing treatment. In this case, the patient is considered to have never demonstrated a response to the agent. This is also referred to a being “refractory” to the treatment. Resistance may also be determined by a treating physician when the platinum agent is no longer considered to be an effective treatment for the cancer.

In some embodiments, the patient is or has been determined to be resistant to treatment with a CDK4/6 inhibitor administered as a monotherapy or in combination with a SOC agent.

CDK4/6 inhibitors in cancer (e.g., a HR⁺ breast cancer as described herein) are known to block entry into S phase of the cell cycle by inducing G1 arrest. Resistance to CDK4/6 inhibitors in cancer (e.g., HR⁺ metastatic breast cancer) has been shown to be mediated, in part, by molecular alterations that: 1) enhance CDK4/6 activity, such as amplifications of CDK6, CCND1, or FGFR1 (Formisano et al., SABCS 2017, Publication Number GS6-05; Cruz et al., SABCS 2017 Publication Number PD4-05), or 2) reactivate cell cycle entry downstream of CDK4/6, such as RB1 loss and CCNE1 amplification (Condorelli, Ann Oncol, PMID: 29236940, 2017; Herrera-Abreu, Cancer Research PMID: 27020857, 2016).

Unlike platinum-based agents which are typically administered for a period of time followed by a period without treatment, CDK4/6 inhibitors, such as palbociclib, ribociclib or abemaciclib, are administered until disease progression is observed. In some instances, a patient will be considered to have become resistant to a CDK4/6 inhibitor when the patient's cancer initially responds or stabilizes while on treatment, but which ultimately begins to progress while still on treatment. In some instances, a patient will be deemed resistant or refractory to treatment with a CDK4/6 inhibitor if the cancer progresses during treatment without demonstrating any significant response or stabilization. Resistance may also be determined by a treating physician when the CDK4/6 inhibitor is no longer considered to be an effective treatment for the cancer.

The methods of the present invention can employ pharmaceutical compositions that include a CDK7 inhibitor described herein or a pharmaceutically acceptable salt thereof and, optionally, a pharmaceutically acceptable carrier. In certain embodiments, the pharmaceutical composition includes a compound of a Formula described herein (generically or specifically) or a pharmaceutically acceptable salt thereof. As noted, a pharmaceutical composition can include one or more pharmaceutically acceptable carriers, and the active agent/ingredient can be provided therein in an effective amount (e.g., a therapeutically effective amount or a prophylactically effective amount). In case of any doubt, any of the CDK7 inhibitors described herein or a pharmaceutically acceptable salt thereof can be included in a pharmaceutical composition of the invention and used in the diagnostic and treatment methods described herein.

Pharmaceutical compositions of the invention can be prepared by relevant methods known in the art of pharmacology. In general, such preparatory methods include the steps of bringing a compound described herein, including a CDK7 inhibitor described herein or a pharmaceutically acceptable salt thereof, into association with a carrier and/or one or more other active ingredients (e.g., a second agent described herein) and/or accessory ingredients, and then, if necessary and/or desirable, shaping and/or packaging the product into a desired single-dose or multi-dose unit (e.g., for oral dosing). The accessory ingredient may improve the bioavailability of a CDK7 inhibitor described herein or a pharmaceutically acceptable salt thereof, may reduce and/or modify its metabolism, may inhibit its excretion, and/or may modify its distribution within the body (e.g., by targeting a diseased tissue (e.g., a tumor). The pharmaceutical compositions can be packaged in various ways, including in bulk containers and as single unit doses (containing, e.g., discrete, predetermined amounts of the active agent) or a plurality thereof, and any such packaged or divided dosage forms are within the scope of the invention. The amount of the active ingredient can be equal to the amount constituting a unit dosage or a convenient fraction of a dosage such as, for example, one-half or one-third of a dose.

Relative amounts of the active agent/ingredient, the pharmaceutically acceptable carrier(s), and/or any additional ingredients in a pharmaceutical composition of the invention can vary, depending upon the identity, size, and/or condition of the subject treated and further depending upon the route by which the composition is to be administered and the disease to be treated. By way of example, the composition may comprise between about 0.1% and 99.9% (w/w or w/v) of an active agent/ingredient.

Pharmaceutically acceptable carriers useful in the manufacture of the pharmaceutical compositions described herein are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable carriers useful in the manufacture of the pharmaceutical compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutical compositions used as described herein may be administered orally. Such orally acceptable dosage forms may be solid (e.g., a capsule, tablet, sachet, powder, granule, and orally dispersible film) or liquid (e.g., an ampoule, semi-solid, syrup, suspension, or solution (e.g., aqueous suspensions or dispersions and solutions). In the case of tablets, carriers commonly used include lactose and corn starch. Lubricating agents, such as magnesium stearate, can also be included. In the case of capsules, useful diluents include lactose and dried cornstarch. When aqueous suspensions are formulated, the active agent/ingredient can be combined with emulsifying and suspending agents. In any oral formulation, sweetening, flavoring or coloring agents may also be added. In any of the various embodiments described herein, an oral formulation can be formulated for immediate release or sustained/delayed release and may be coated or uncoated. A provided composition can also be micro-encapsulated.

Compositions suitable for buccal or sublingual administration include tablets, lozenges and pastilles. Formulations can also be prepared for subcutaneous, intravenous, intramuscular, intraocular, intravitreal, intra-articular, intra-synovial, intrasternal, intrathecal, intrahepatic, intraperitoneal intralesional and by intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Although the descriptions of pharmaceutical compositions provided herein are principally directed to pharmaceutical compositions which are suitable for administration to humans, it will be understood by one of ordinary skill in the art that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification.

Compounds described herein are typically formulated in dosage unit form, e.g., single unit dosage form, for ease of administration and uniformity of dosage. The specific therapeutically or prophylactically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount can vary from subject to subject, depending, for example, on species, age, and general condition of a subject, severity of the side effects, disease to be treated, identity of the particular compound(s) to be administered, mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a CDK7 inhibitor for administration one or more times a day (e.g., once) to an adult human (e.g., 70 kg) may comprise about 1-100 mg, about 1-50 mg, about 1-35 mg (e.g., about 1-5, 1-10, 1-15, 1-20, 1-25, or 1-30 mg), about 2-20 mg, about 3-15 mg or about 10-30 mg (e.g., 10-20 or 10-25 mg). Here, and wherever ranges are referenced, the end points are included. The dosages provided here can be scaled for patients of differing weights or body surface and may be expressed per m² of the patient's body surface.

In certain embodiments, compositions of the invention may be administered once per day. The dosage of a CDK7 inhibitor described herein or a pharmaceutically acceptable salt thereof can be about 1-100 mg, about 1-50 mg, about 1-25 mg, about 2-20 mg, about 5-15 mg, about 10-15 mg, or about 13-14 mg.

In certain embodiments, a composition of the invention may be administered twice per day. In some embodiments, the dosage of a compound of Formula (I) or a subgenus or species thereof for each administration is about 0.5 mg to about 50 mg, about 0.5 mg to about 25 mg, about 0.5 mg to about 1 mg, about 1 mg to about 10 mg, about 1 mg to about 5 mg, about 3 mg to about 5 mg, or about 4 mg to about 5 mg.

A compound or other composition described herein (e.g., a pharmaceutical composition comprising a CDK7 inhibitor) can be administered in a combination therapy (e.g., as defined and further described herein) with a second agent described herein or a plurality thereof. The additional/second agent employed in a combination therapy is most likely to achieve a desired effect for the same disorder (e.g., the same cancer), however it may achieve different effects that aid the patient. Accordingly, the invention features pharmaceutical compositions containing a CDK7 inhibitor described herein, or a pharmaceutically acceptable salt thereof, in a therapeutically effect amount; a second agent selected from a Bcl-2 inhibitor such as venetoclax, a PARP inhibitor such as olaparib or niraparib, a platinum-based anti-cancer agent such as carboplatin, cisplatin, or oxaliplatin, a taxane such as paclitaxel, a CDK4/6 inhibitor such as palbociclib, ribociclib, abemaciclib, or trilaciclib, a selective estrogen receptor modulator (SERM) such as tamoxifen (available under the brand names Nolvadex™ and Soltamox™) raloxifene (available under the brand name Evista™), and toremifene (available as Fareston™) and a selective estrogen receptor degrader such as fulvestrant (available as Faslodex™), each in a therapeutically effective amount; and a pharmaceutically acceptable carrier.

An identified patient can be “newly diagnosed” and therefor previously unexposed to a second agent as described herein, in which case the patient may be defined as treatment naïve.

Unless otherwise specified, when employing a combination of a CDK7 inhibitor described herein or a pharmaceutically acceptable salt thereof and a second therapeutic agent in a method of the invention, the second therapeutic agent can be administered concurrently with, prior to, or subsequent to a CDK7 inhibitor described herein or a pharmaceutically acceptable salt thereof. The second therapeutic pharmaceutical agent may be administered at a dose and/or on a time schedule determined for that pharmaceutical agent. The second therapeutic agent may also be administered together with a CDK7 inhibitor described herein or a pharmaceutically acceptable salt thereof, in a single dosage form or administered separately in different dosage forms. In general, it is expected that the second therapeutic agents utilized in combination with a CDK7 inhibitor described herein or a pharmaceutically acceptable salt thereof, will be utilized at levels that do not exceed the levels at which they are utilized individually. In some embodiments, the levels of the second therapeutic agent utilized in combination will be lower than those utilized in a monotherapy due to synergistic effects.

For combinations of a CDK7 inhibitor described herein, or a pharmaceutically acceptable salt thereof, and an additional/second agent selected from any one of those described herein, a kit comprising each of the two active therapeutics (or more, e.g., further including a third agent) can be provided and is within the scope of the present invention. Such kits find utility in any of the diagnostic and treatment methods described herein. In some instances, the first and second agents will be in separate vessels (e.g., with the first agent confined to a first container and the second agent confined to a second container) and/or formulated in a pharmaceutically acceptable composition, optionally in unit dosage form, that includes the first agent, the second agent, and a pharmaceutically acceptable carrier. In some instances, the kits include a written insert or label with instructions to use the two (or more) therapeutic agents in a patient suffering from a cancer (e.g., as described herein) and identified as amenable to treatment by a method described herein. The instructions may be adhered or otherwise attached to a vessel or vessels comprising the therapeutic agents. Alternatively, the instructions and the vessel(s) can be separate from one another but present together in a single kit, package, box, bag, or other type of container. The instructions in the kit will typically be mandated or recommended by a governmental agency approving the therapeutic use of the combination (e.g., in a patient population identified as described herein). The instructions may optionally comprise dosing information for each therapeutic agent, the types of cancer for which treatment of the combination was approved or may be prescribed, physicochemical information about each of the therapeutics, pharmacokinetic information about each of the therapeutics, drug-drug interaction information, or diagnostic information (e.g., based on a biomarker or a method of identifying a patient for treatment as described herein). The kits of the invention can also include reagents useful in the diagnostic methods described herein.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

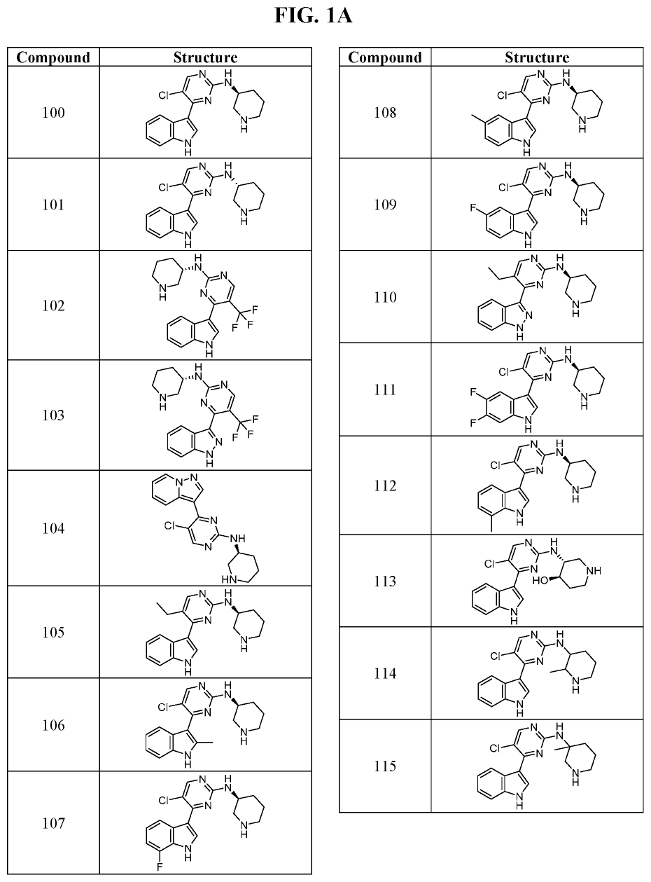

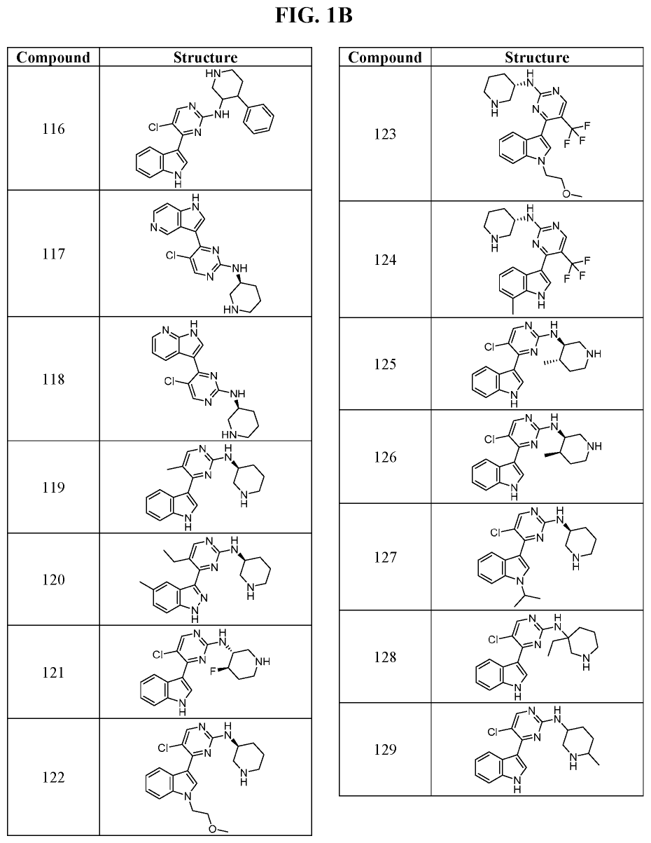

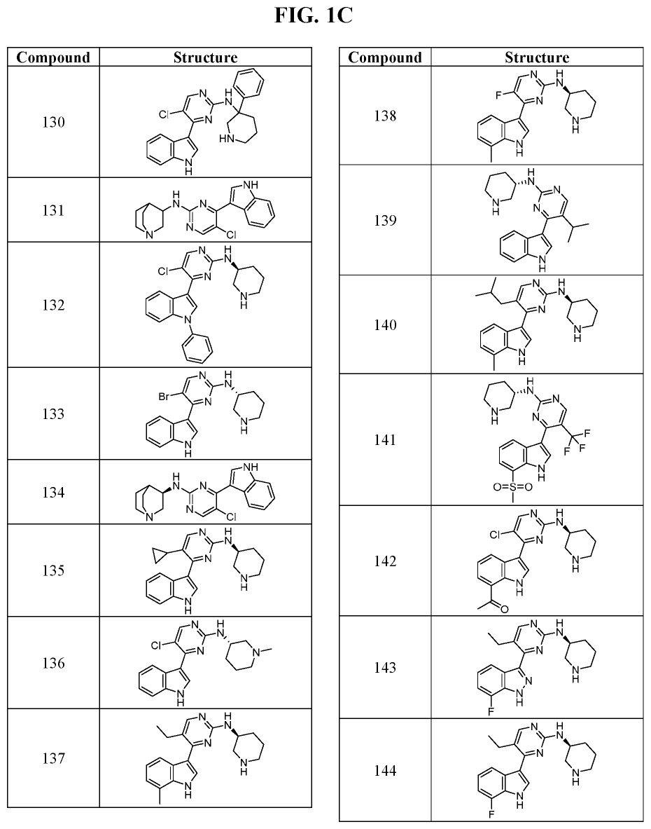

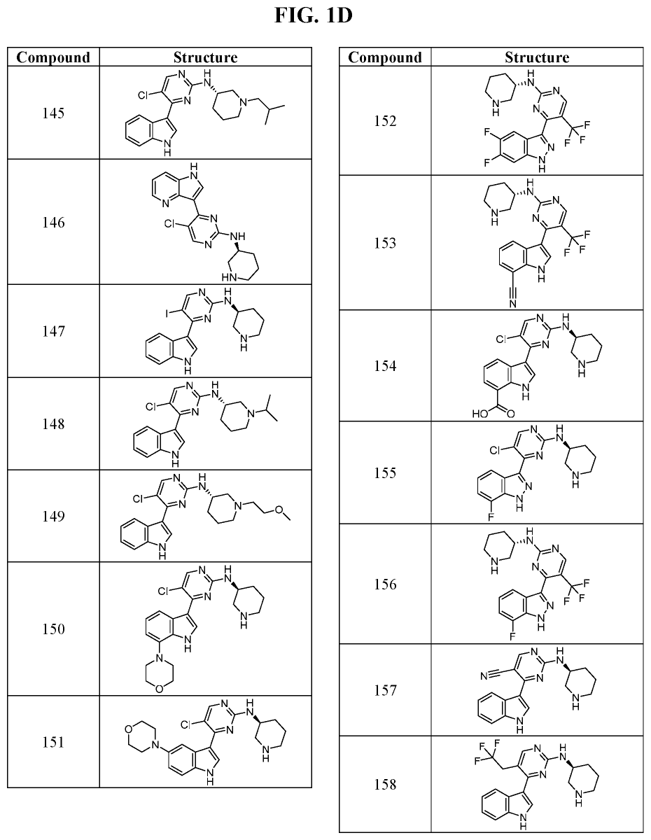

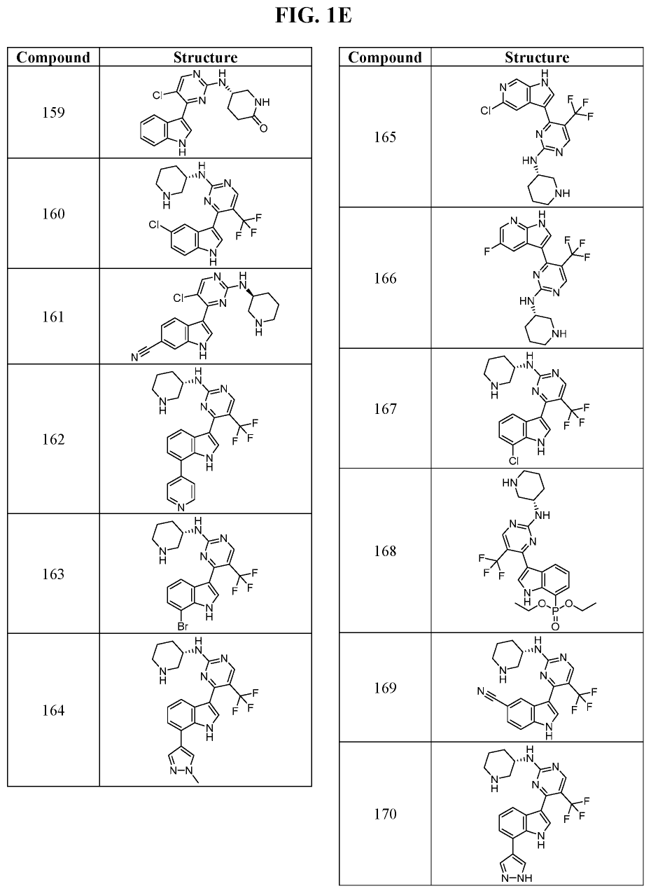

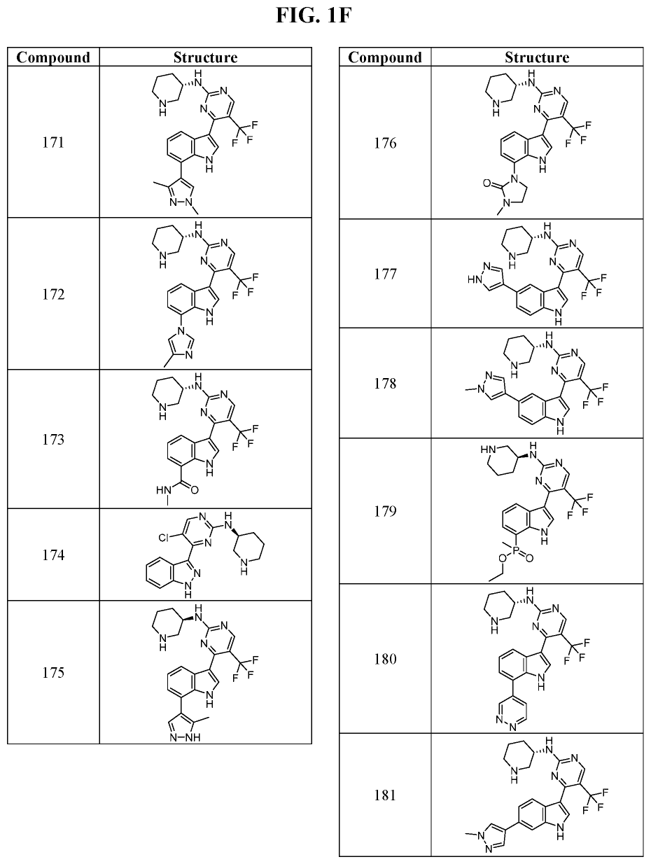

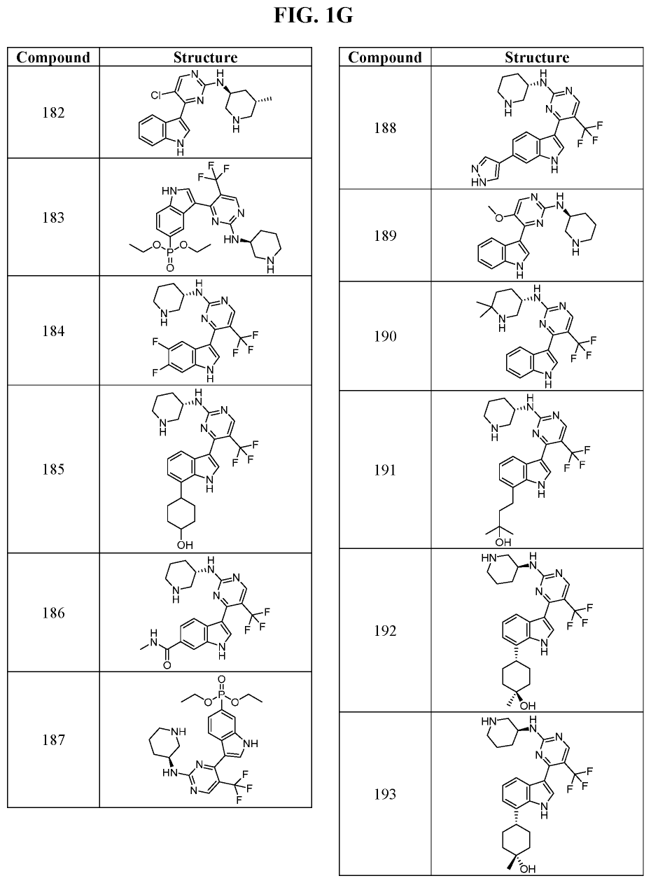

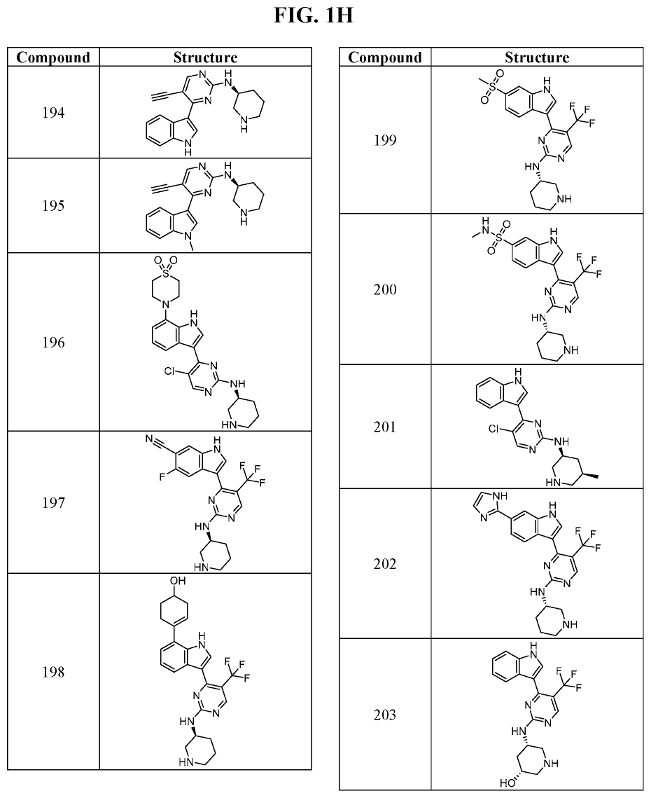

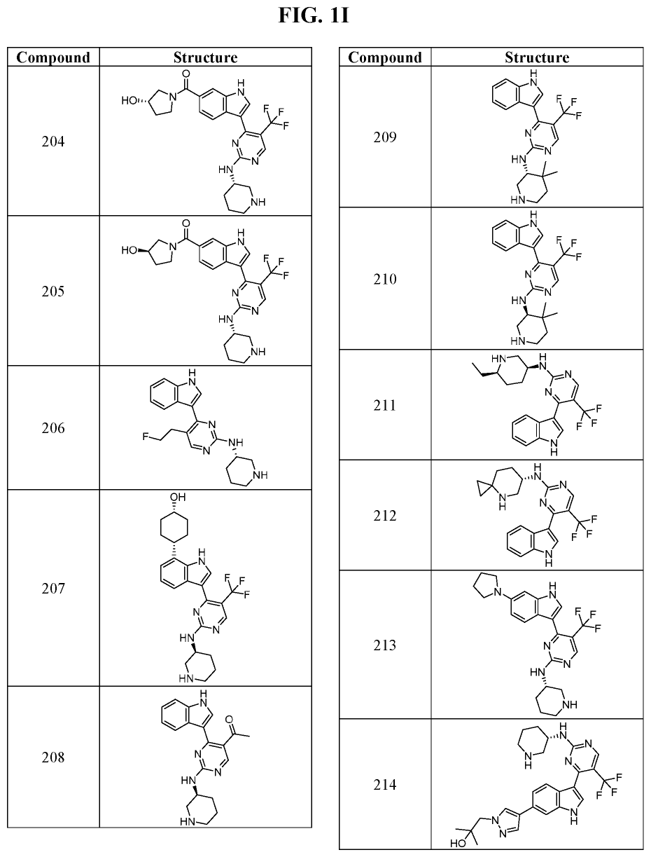

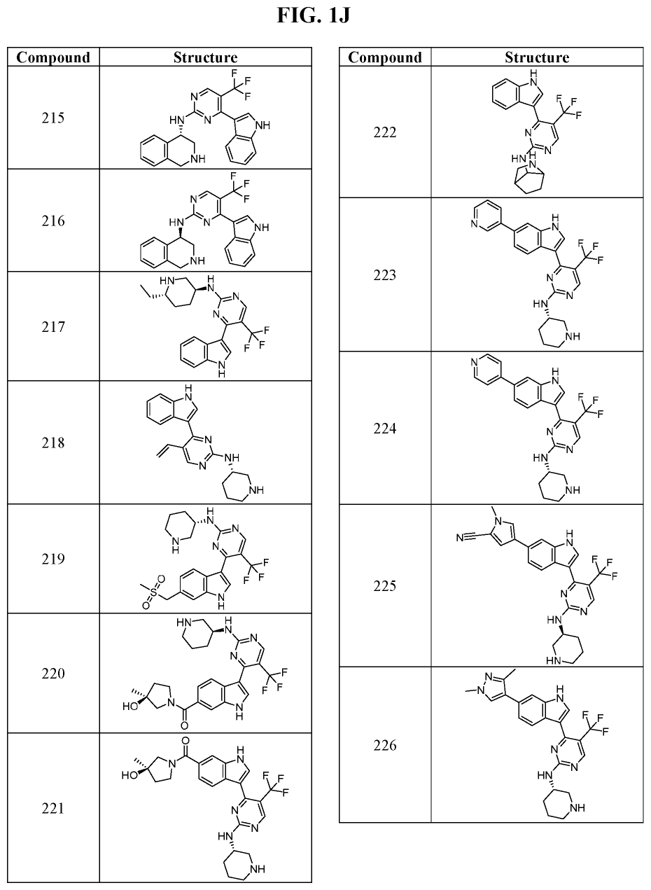

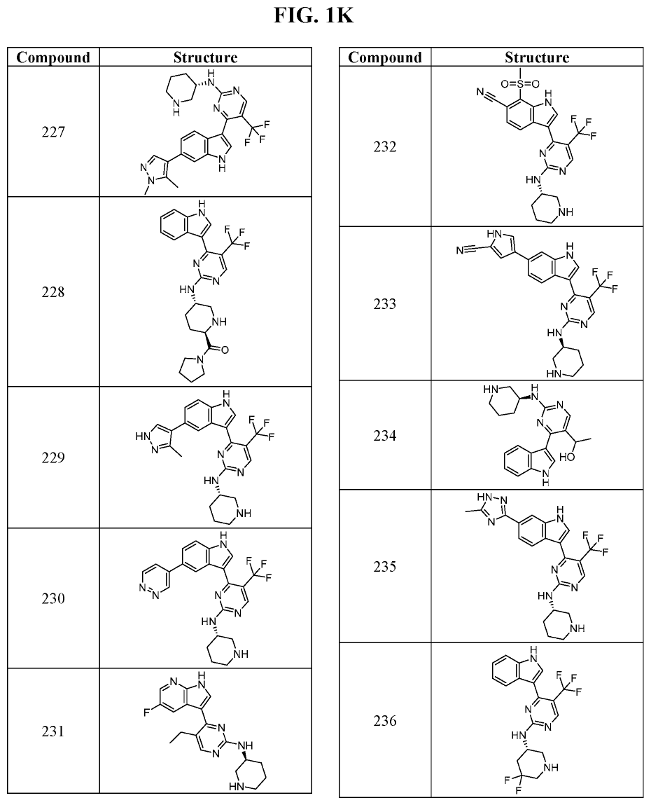

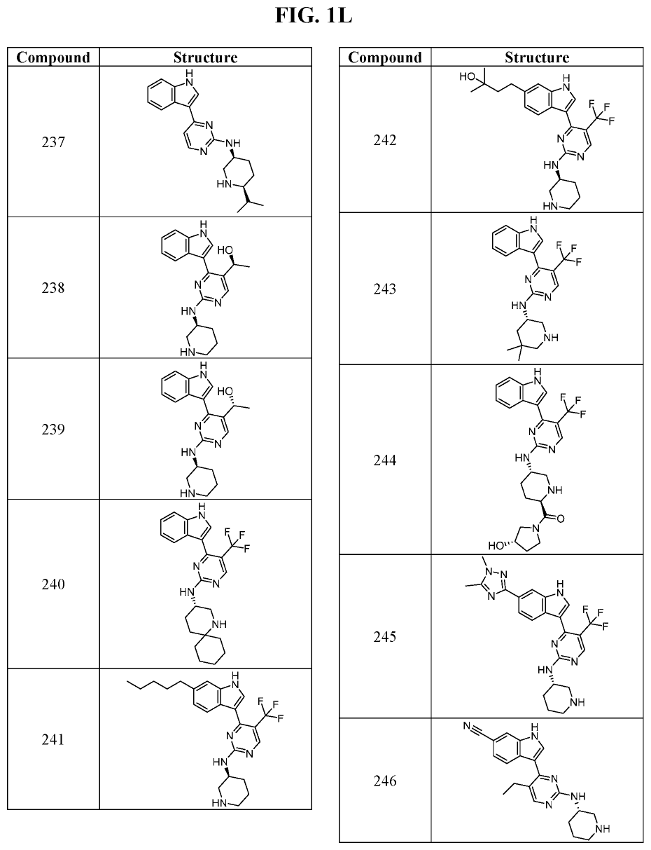

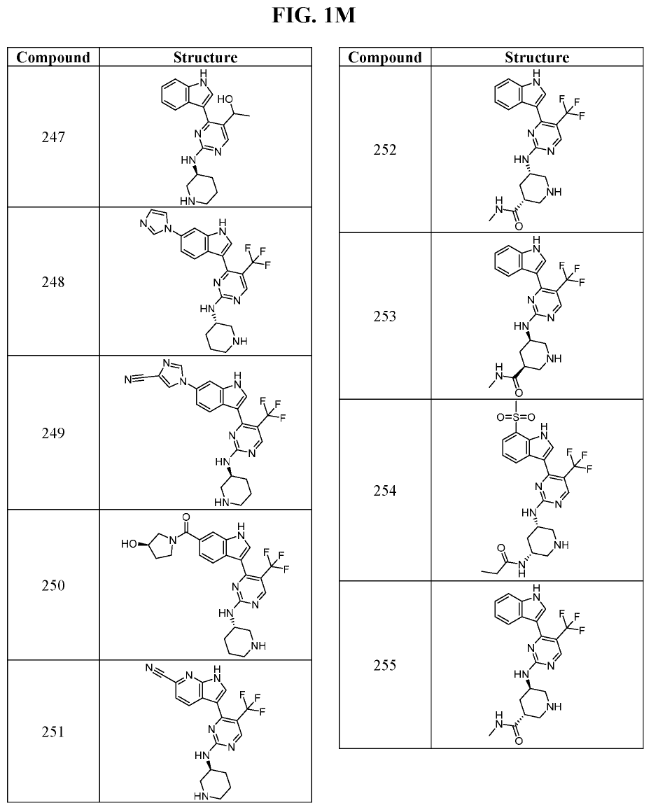

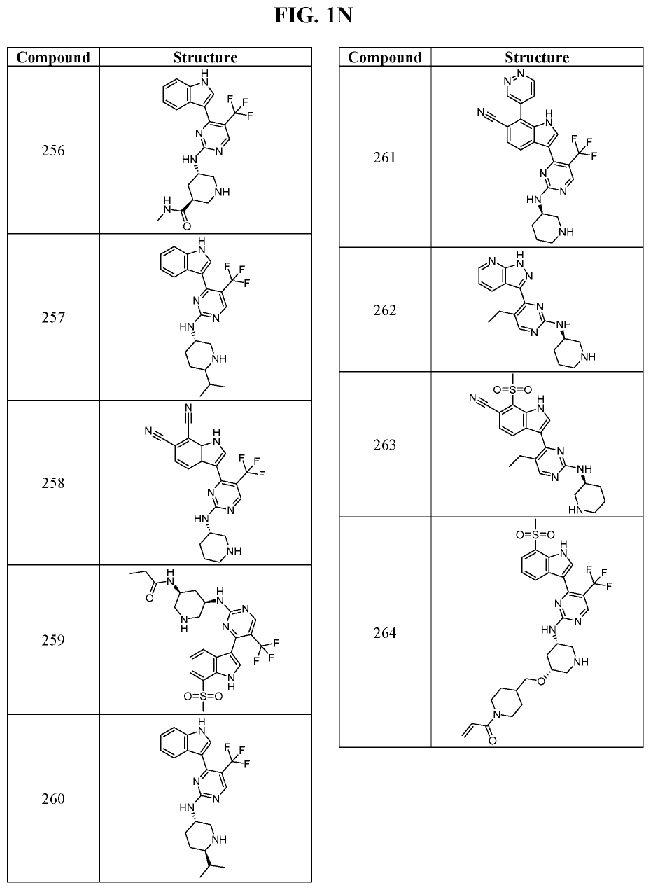

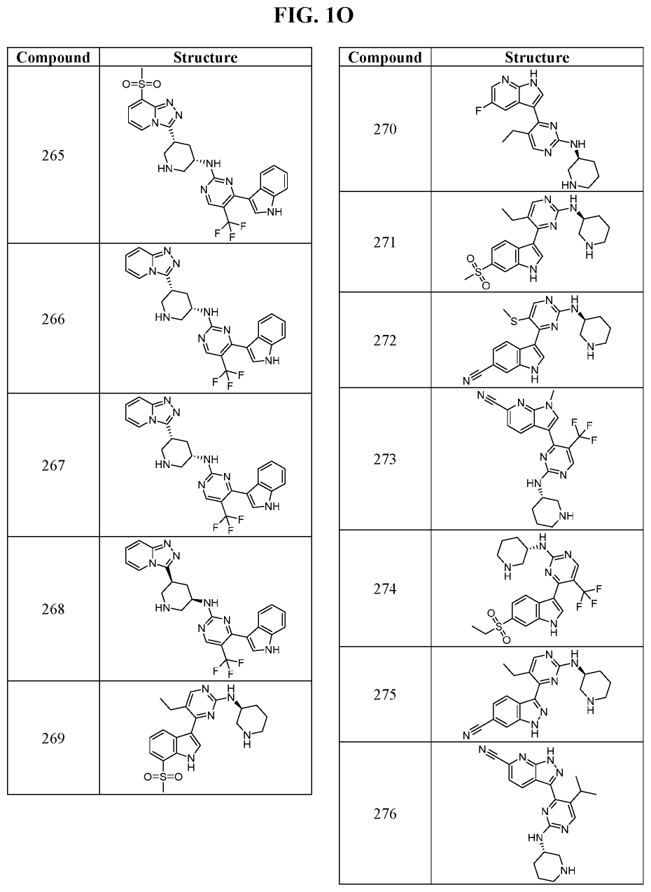

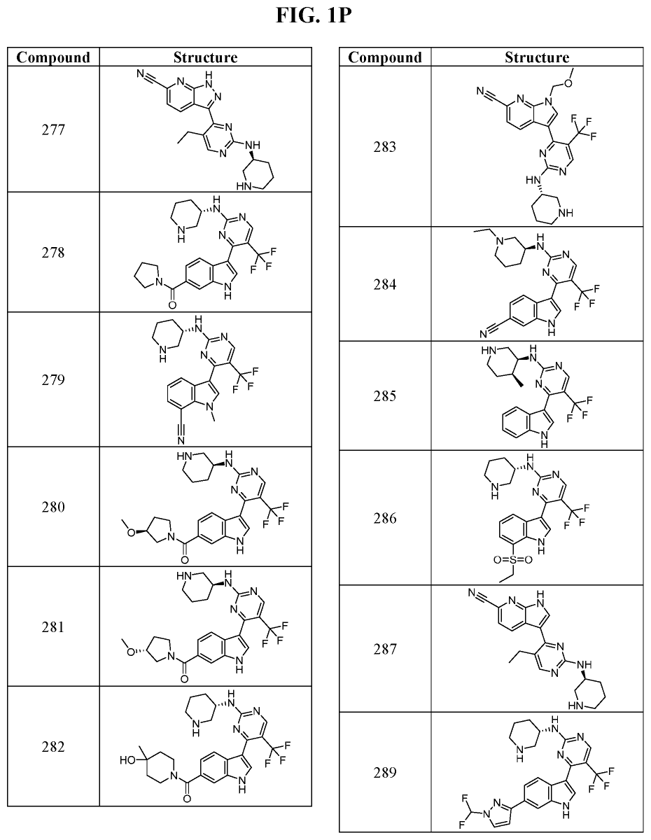

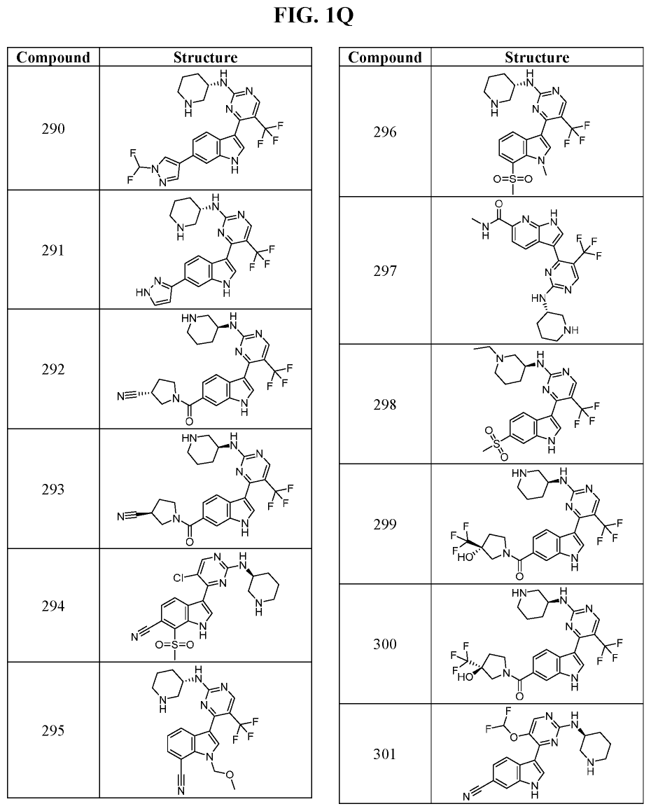

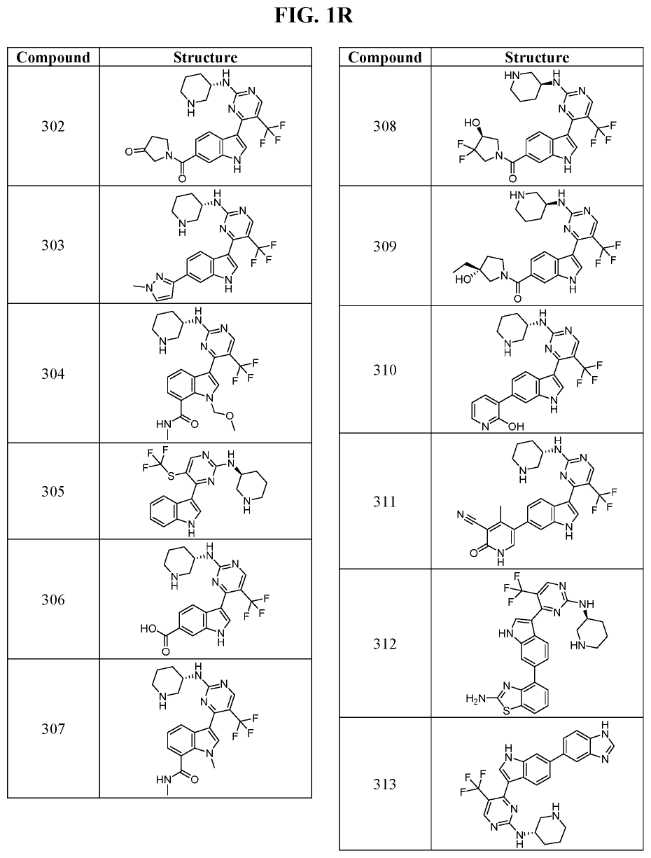

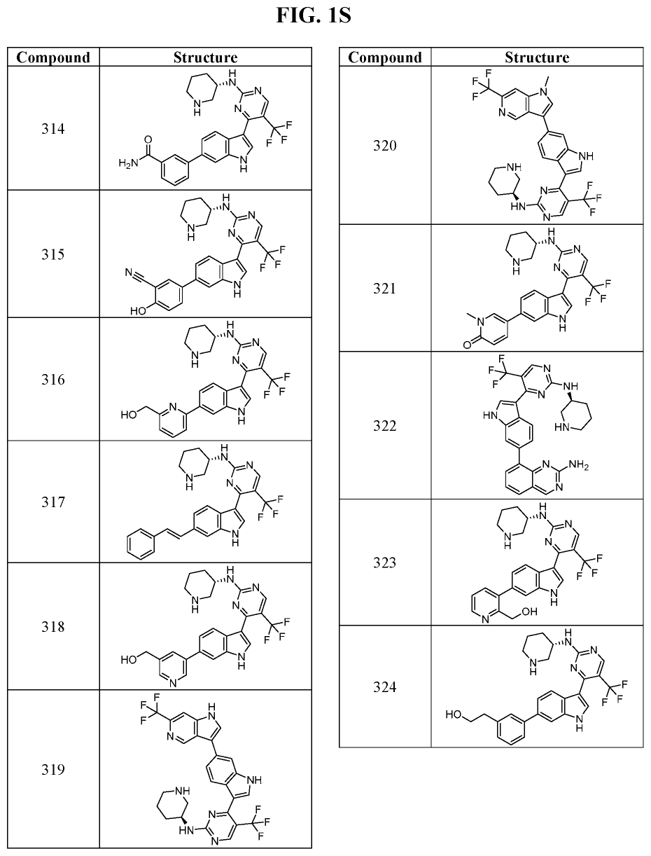

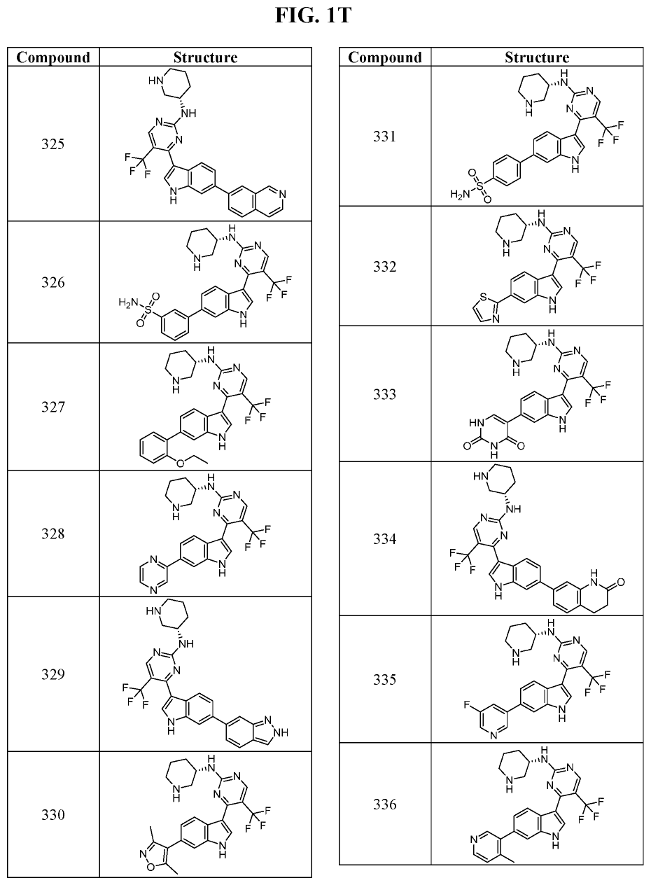

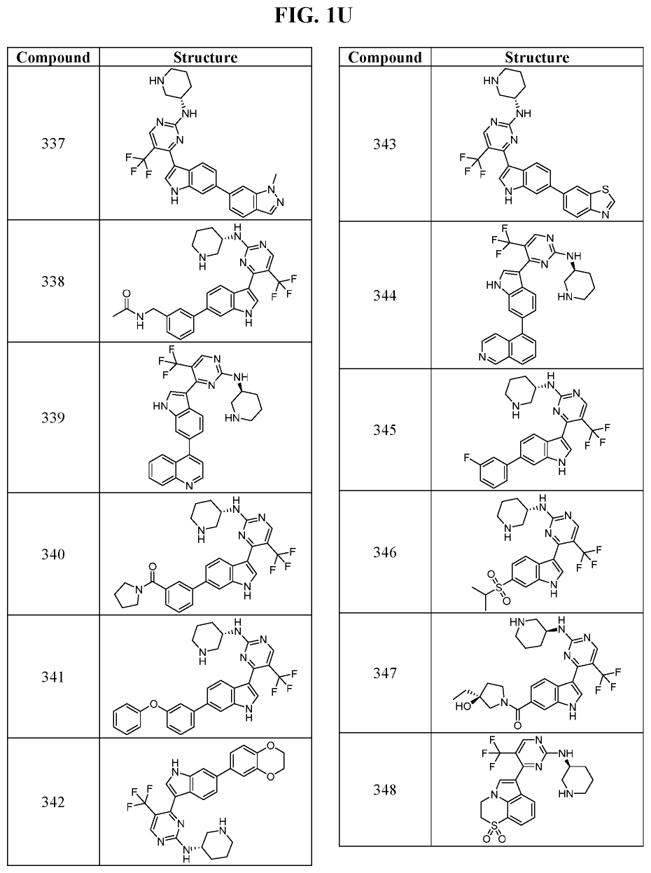

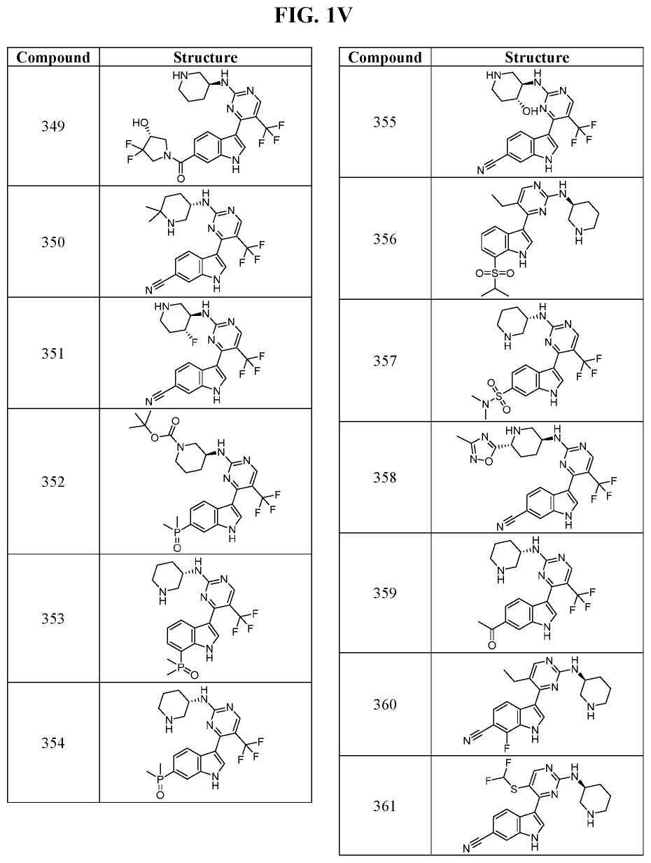

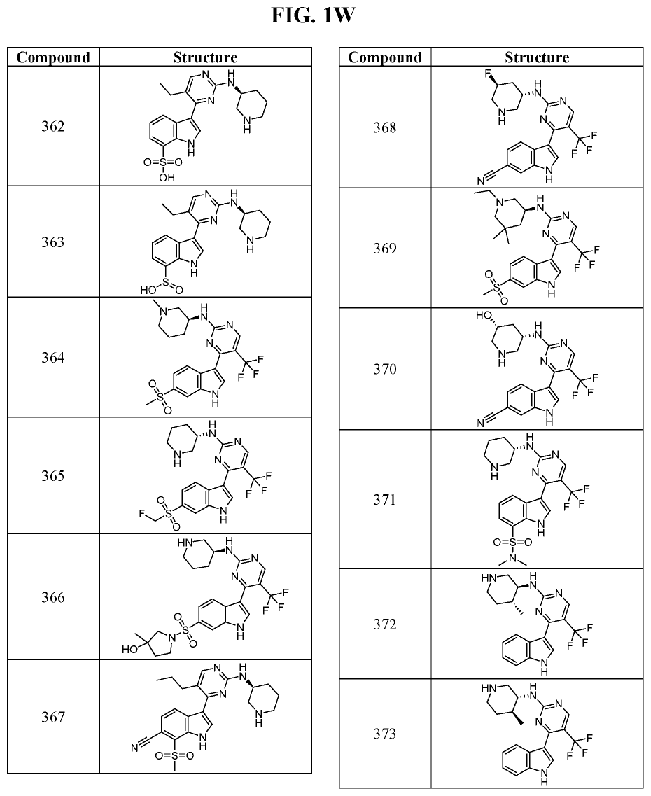

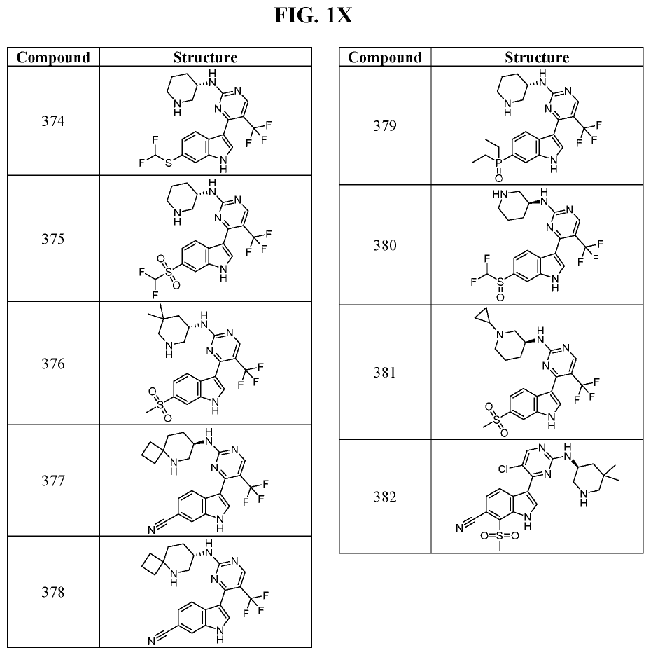

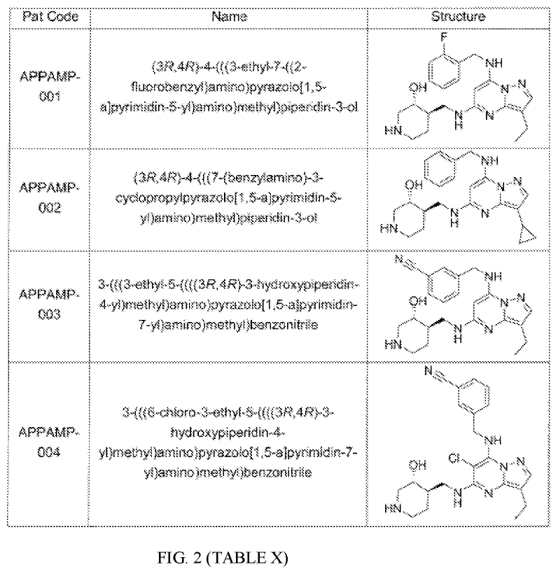

Syntheses of exemplary compounds from FIG. 1A-IX are described in WO2018/013867.

### Example 1: Inhibition of CDK Kinase Activity

Compounds of the invention were assayed for inhibition of CDK7, CDK9, CDK12, and CDK2 activity at Biortus Biosciences (Jiangyin, Jiangsu Province, P.R. of China) using kinase assays for each CDK developed with a Caliper/LabChip EZ Reader (Perkin Elmer, Waltham, Mass.). These assays measure the amount of phosphorylated peptide substrate produced as a fraction of the total peptide following an incubation period at 27° C. with the following components: test compounds (variable concentrations from 10 μM down to 0.508 nM in a series of 3-fold serial dilutions), active CDK kinase protein (with the indicated Cyclin, listed below for each CDK), ATP (2 mM), and substrate peptide (listed below) in the following buffer: 2-(N-morpholino)ethanesulfonate (MES buffer, 20 mM), pH 6.75, 0.01% (v/v) Tween 20 detergent, 0.05 mg/mL bovine serum albumin (BSA).

Specifically, the CDK7 inhibition assay used CDK7/Cyclin H/MAT1 complex (6 nM) and “5-FAM-CDK7tide” peptide substrate (2 μM, synthesized fluorophore-labeled peptide with the following sequence: 5-FAM-YSPTSPSYSPTSPSYSPTSPSKKKK (SEQ ID NO:1), where “5-FAM” means 5-carboxyfluorescein) with 6 mM MgCl2 in the buffer composition listed above. Furthermore, the CDK9 inhibition assay used CDK9/Cyclin T1 complex (8 nM) and “5-FAM-CDK9tide” peptide substrate (2 μM, synthesized fluorophore-labeled peptide with the following sequence: 5-FAM-GSRTPMY-NH₂ where 5-FAM is defined above and NH₂ signifies a C-terminal amide) with 10 mM MgCl2 in the buffer composition listed above. The CDK12 inhibition assay used CDK12 (aa686-1082)/Cyclin K complex (50 nM) and “5-FAM-CDK9tide” (2 μM) as defined above, with 2 mM MgCl2 in the buffer composition above. Additionally, the CDK2 inhibition assay used CDK2/Cyclin E1 complex (0.5 nM) and “5-FAM-CDK7tide” (2 μM) as defined above, with 2 mM MgCl2 in the buffer composition listed above.

The incubation period at 27° C. for each CDK inhibition assay was chosen such that the fraction of phosphorylated peptide product produced in each assay, relative to the total peptide concentration, was approximately 20% (±5%) for the uninhibited kinase (35 min. for CDK7, 35 min. for CDK2, 3 hr. for CDK12, 15 min. for CDK9). In cases where the compound titrations were tested and resulted in inhibition of peptide product formation, these data were fit to produce best-fit IC₅₀ values. The results of these assays are shown below in Table 1 where “A” represents a calculated IC₅₀ of less than 20 nM; “B” represents a calculated IC₅₀ of between 20 nM and less than 200 nM; “C” represents a calculated IC₅₀ of between 200 nM and less than 5 μM; “D” represents a calculated IC₅₀ of greater than or equal to 5 μM, and “NT” represents that the specified compound was not tested in the specified assay.

TABLE 1
Inhibitory Activity of Selected Compounds of the
Invention Against CDK2, CDK7, CDK9, and CDK12.

| Compound | CDK2 | CDK7 | CDK9 | CDK12 |
|---|---|---|---|---|
| 100 | C | A | C | C |
| 101 | C | C | C | C |
| 102 | C | A | B | B |
| 103 | C | A | C | B |
| 104 | C | B | C | B |
| 105 | D | B | D | C |
| 106 | D | B | C | C |
| 107 | C | B | C | B |
| 108 | C | B | C | C |
| 109 | C | A | C | B |
| 110 | C | B | C | C |
| 111 | C | A | B | B |
| 112 | C | A | C | B |
| 113 | D | B | D | C |
| 114 | C | B | C | C |
| 115 | D | C | D | D |
| 116 | D | D | D | D |
| 117 | C | B | C | C |
| 118 | C | A | C | B |
| 119 | D | B | C | C |
| 120 | D | C | D | C |
| 121 | C | B | C | C |
| 122 | D | C | C | D |
| 123 | D | B | C | D |
| 124 | C | A | C | B |
| 125 | D | B | D | D |
| 126 | C | B | C | C |
| 127 | C | C | C | D |
| 128 | D | D | D | D |
| 129 | NT | B | NT | C |
| 130 | NT | D | NT | D |
| 131 | D | D | D | D |
| 132 | D | C | C | D |
| 133 | B | A | C | B |
| 134 | D | D | D | D |
| 135 | D | B | D | D |
| 136 | C | A | C | C |
| 137 | C | A | C | C |
| 138 | C | B | C | C |
| 139 | D | B | D | D |
| 140 | D | C | D | D |
| 141 | C | A | C | C |
| 142 | C | A | C | C |
| 143 | C | A | C | C |
| 144 | C | A | C | C |
| 145 | D | B | D | C |
| 146 | D | D | D | D |
| 147 | B | A | C | C |
| 148 | D | B | C | C |
| 149 | C | B | C | C |
| 150 | C | B | C | C |
| 151 | D | C | D | D |
| 152 | C | A | C | C |
| 153 | B | A | C | B |
| 154 | D | B | C | C |
| 155 | C | B | C | C |
| 156 | C | A | C | C |
| 157 | C | A | C | C |
| 158 | D | C | D | C |
| 159 | B | B | C | C |
| 160 | C | A | C | C |
| 161 | C | A | C | C |
| 162 | NT | B | C | C |
| 163 | NT | B | C | C |
| 164 | NT | B | C | C |
| 165 | C | A | C | C |
| 166 | B | A | C | C |
| 167 | C | A | C | B |
| 168 | D | C | D | D |
| 169 | D | B | D | C |
| 170 | C | A | C | B |
| 171 | C | A | C | C |
| 172 | C | A | C | C |
| 173 | C | A | C | C |
| 174 | C | A | C | C |
| 175 | C | A | B | C |
| 176 | C | A | C | C |
| 177 | D | B | D | C |
| 178 | D | B | D | C |
| 179 | C | A | C | C |
| 180 | B | A | C | B |
| 181 | C | A | C | C |
| 182 | D | B | C | C |
| 183 | C | B | C | D |
| 184 | B | A | B | B |
| 185 | B | A | C | C |
| 186 | C | A | C | C |
| 187 | D | B | D | C |
| 188 | C | A | C | C |
| 189 | D | C | D | D |
| 190 | C | A | C | C |
| 191 | C | A | C | C |
| 192 | C | B | C | C |
| 193 | C | B | C | C |
| 194 | C | A | C | C |
| 195 | D | C | D | D |
| 196 | C | A | C | C |
| 197 | C | A | C | C |
| 198 | C | A | C | C |
| 199 | C | A | C | C |
| 200 | C | A | C | C |
| 201 | C | A | C | B |
| 202 | C | A | C | D |
| 203 | C | A | C | C |
| 204 | D | A | D | C |
| 205 | D | A | D | C |
| 206 | D | B | C | C |
| 207 | C | A | C | C |
| 208 | D | B | D | C |
| 209 | D | C | D | D |
| 210 | D | C | D | D |
| 211 | C | B | C | C |
| 212 | C | A | C | C |
| 213 | D | B | D | C |
| 214 | C | B | C | C |
| 215 | D | B | D | D |
| 216 | C | C | D | D |
| 217 | C | A | C | C |
| 218 | C | A | C | C |
| 219 | C | A | C | C |
| 220 | D | A | D | C |
| 221 | D | A | D | D |
| 222 | C | B | C | C |
| 223 | C | A | C | C |
| 224 | C | A | C | C |
| 225 | C | A | C | C |
| 226 | C | A | C | C |
| 227 | C | A | C | C |
| 228 | C | A | C | C |
| 229 | C | A | C | C |
| 230 | C | A | C | C |
| 231 | D | D | D | D |
| 232 | C | A | C | C |
| 233 | C | A | C | C |
| 234 | D | C | D | D |
| 235 | C | A | C | C |
| 236 | C | A | C | C |
| 237 | C | B | D | C |
| 238 | D | C | D | D |
| 239 | D | C | D | D |
| 240 | C | B | C | C |
| 241 | C | B | C | C |
| 242 | D | B | C | C |
| 243 | C | A | C | B |
| 244 | C | B | D | C |
| 245 | C | A | D | C |
| 246 | D | A | D | D |
| 247 | D | C | D | D |
| 248 | C | A | C | C |
| 249 | D | A | D | C |
| 250 | C | A | D | C |
| 251 | C | A | C | C |
| 252 | C | B | D | D |
| 253 | B | A | C | B |
| 254 | D | C | D | D |
| 255 | C | A | C | C |
| 256 | C | B | D | C |
| 257 | D | B | D | D |
| 258 | C | A | C | C |
| 259 | C | A | C | C |
| 260 | D | C | D | C |
| 261 | C | B | C | C |
| 262 | C | A | C | C |
| 263 | D | A | D | D |
| 264 | D | B | D | C |
| 265 | C | C | C | C |
| 266 | C | B | C | C |
| 267 | C | B | C | C |
| 274 | C | A | C | C |
| 275 | C | A | C | C |
| 276 | D | B | D | D |
| 277 | C | A | C | C |
| 278 | C | B | D | C |
| 279 | D | B | D | D |
| 280 | D | B | D | C |
| 281 | C | A | C | C |
| 282 | C | A | D | C |
| 283 | D | C | D | D |
| 284 | C | A | C | C |
| 285 | D | C | D | D |
| 286 | C | A | C | C |
| 287 | D | A | D | D |
| 289 | C | B | C | C |
| 290 | C | B | C | C |
| 291 | C | A | C | C |
| 292 | C | A | C | C |
| 293 | C | A | C | C |
| 294 | C | A | C | C |
| 295 | D | B | D | D |
| 296 | D | B | D | D |
| 297 | C | A | C | C |
| 298 | C | A | D | C |
| 299 | C | A | C | C |
| 300 | C | A | C | C |
| 301 | D | B | D | D |
| 302 | C | B | C | C |
| 303 | C | A | C | C |
| 304 | D | D | D | D |
| 305 | C | A | C | C |
| 306 | C | B | D | C |
| 307 | D | D | D | D |
| 308 | C | A | C | C |
| 309 | D | B | D | C |
| 310 | C | A | D | C |
| 311 | C | A | C | C |
| 312 | NT | B | NT | C |
| 313 | C | A | C | C |
| 314 | C | A | C | C |
| 315 | NT | B | NT | C |
| 316 | C | A | D | C |
| 317 | NT | B | NT | C |
| 318 | C | A | C | C |
| 319 | NT | B | NT | C |
| 320 | NT | B | NT | C |
| 321 | D | A | D | C |
| 322 | NT | A | NT | C |
| 323 | C | A | D | C |
| 324 | NT | B | NT | C |
| 325 | NT | B | NT | C |
| 326 | NT | B | NT | C |
| 327 | NT | B | NT | C |
| 328 | C | A | C | C |
| 329 | NT | B | NT | C |
| 330 | C | A | C | C |
| 331 | NT | B | NT | C |
| 332 | C | A | C | C |
| 333 | D | A | D | C |
| 334 | C | B | C | C |
| 335 | C | B | C | C |
| 336 | C | A | C | C |
| 337 | NT | B | NT | C |
| 338 | C | B | C | C |
| 339 | D | B | D | C |
| 340 | C | B | C | C |
| 341 | D | C | D | D |
| 342 | D | B | D | C |
| 343 | C | B | C | C |
| 344 | C | B | D | C |
| 345 | D | C | D | D |
| 346 | C | B | C | C |
| 347 | D | B | D | C |
| 348 | D | B | D | D |
| 349 | C | A | C | C |
| 350 | D | B | D | C |
| 351 | C | A | C | C |
| 352 | D | D | D | D |
| 353 | C | A | D | C |
| 354 | D | B | D | C |
| 355 | D | B | D | C |
| 356 | D | B | D | D |
| 357 | C | A | C | C |
| 358 | D | B | D | D |
| 359 | C | A | C | C |
| 360 | D | B | D | D |
| 361 | D | B | C | C |
| 362 | D | B | D | D |
| 363 | D | B | D | D |
| 364 | D | B | D | D |
| 365 | C | A | C | C |
| 366 | C | B | C | C |
| 367 | D | B | D | D |
| 368 | D | B | D | C |
| 369 | D | B | D | D |
| 370 | D | B | D | C |
| 371 | C | A | C | C |
| 372 | C | B | C | C |
| 373 | D | B | D | D |
| 374 | C | A | C | B |
| 375 | C | A | C | C |
| 376 | C | B | D | C |
| 377 | D | A | C | C |
| 378 | D | B | D | D |
| 379 | D | B | D | D |
| 380 | C | A | C | C |
| 381 | D | B | D | C |
| 382 | C | A | C | C |

### Example 2. Inhibition of Cell Proliferation

A673 Cells: A673 cells are a cell line derived from human muscle Ewing's sarcoma. Representative compounds of the invention were tested at different concentrations (from 4 μM to 126.4 μM; 0.5 log serial dilutions) for their ability to inhibit the proliferation of A673 cells. Known CDK inhibitors dinaciclib or N-((1S,3R)-3-((5-chloro-4-(1H-indol-3-yl)pyrimidin-2-yl)amino)cyclohexyl)-5-((E)-4-(dimethylamino)but-2-enamido)picolinamide and triptolide were used as positive controls. Cells were grown in Dulbecco's Modified Eagle's Medium, +10% FBS+1 mM Sodium Pyruvate. The cells were cultured at 37° C. in a humidified chamber in the presence of 5% CO₂. Proliferation assays were conducted over a 72 hour time period. CyQUANT® (Life Technologies, Chicago, Ill. USA) was used to assess the anti-proliferative effects of the compounds following manufacturer's directions and utilizing the reagents supplied with the CyQUANT® kit. The results of the assay are shown below in Table 1 where “A” represents a calculated IC₅₀ of less than 20 nM; “B” represents a calculated IC₅₀ of between 20 nM and less than 200 nM; “C” represents a calculated IC₅₀ of 200 nM and less than 5 μM; “D” represents a calculated IC₅₀ of greater than 5 μM.

HCC70 cells: HCC70 cells are a cell line derived from human triple negative breast cancer. Representative compounds of the invention were tested at different concentrations (from 4 μM to 126.4 μM; 0.5 log serial dilutions) for their ability to inhibit the proliferation of HCC70 cells. Known CDK inhibitors dinaciclib or N-((1S,3R)-3-((5-chloro-4-(1H-indol-3-yl) pyrimidin-2-yl)amino)cyclohexyl)-5-((E)-4-(dimethylamino)but-2-enamido)picolinamide and triptolide were used as positive controls. Cells were grown in ATCC-formulated RPMI-1640 Medium (ATCC 30-2001)+10% FBS. The cells were cultured at 37° C. in a humidified chamber in the presence of 5% CO₂. Proliferation assays were conducted over a 72 hour time period. CyQUANT® Direct Cell Proliferation Assay (Life Technologies, Chicago, Ill. USA) was used to assess the anti-proliferative effects of the compounds following manufacturer's directions and utilizing the reagents supplied with the CyQUANT® Direct Cell kit. The results of the assay are shown below in Table 2 where “A” represents a calculated IC₅₀ of less than 100 nM; “B” represents a calculated IC₅₀ of between 100 nM and less than 1000 nM; “C” represents a calculated IC₅₀ of between 1000 nM and less than 5 μM; “D” represents a calculated IC₅₀ of greater than 5 μM.

TABLE 2
Inhibition of Proliferation of A673 Cells and HCC70
Cells by Compounds of the invention.

| Compound | A673 Cells | HCC70 Cells |
|---|---|---|
| 100 | A | NT |
| 101 | B | NT |
| 102 | A | NT |
| 103 | B | A |
| 104 | B | B |
| 105 | B | NT |
| 106 | B | NT |
| 107 | A | B |
| 108 | C | NT |
| 109 | B | B |
| 110 | B | NT |
| 111 | B | NT |
| 112 | A | A |
| 113 | C | NT |
| 114 | B | NT |
| 115 | C | NT |
| 116 | C | NT |
| 117 | B | NT |
| 118 | B | A |
| 119 | B | NT |
| 120 | NT | NT |
| 121 | B | NT |
| 122 | C | NT |
| 123 | C | NT |
| 124 | A | NT |
| 125 | C | NT |
| 126 | NT | NT |
| 127 | C | NT |
| 128 | NT | NT |
| 129 | B | NT |
| 130 | C | NT |
| 131 | C | NT |
| 132 | C | NT |
| 133 | A | NT |
| 134 | C | NT |
| 135 | C | NT |
| 136 | B | NT |
| 137 | B | NT |
| 138 | C | NT |
| 139 | C | C |
| 140 | C | NT |
| 141 | A | A |
| 142 | A | NT |
| 143 | A | NT |
| 144 | B | B |
| 145 | C | NT |
| 146 | B | NT |
| 147 | A | NT |
| 148 | B | NT |
| 149 | B | NT |
| 150 | B | NT |
| 151 | B | NT |
| 152 | A | NT |
| 153 | A | NT |
| 154 | C | NT |
| 155 | A | NT |
| 156 | A | NT |
| 157 | A | NT |
| 158 | B | NT |
| 159 | C | C |
| 160 | A | NT |
| 161 | A | A |
| 162 | A | NT |
| 163 | B | NT |
| 164 | A | NT |
| 165 | B | B |
| 166 | A | A |
| 167 | A | NT |
| 168 | C | NT |
| 169 | C | NT |
| 170 | A | A |
| 171 | A | NT |
| 172 | A | NT |
| 173 | B | B |
| 174 | B | NT |
| 175 | A | C |
| 176 | A | B |
| 177 | C | NT |
| 178 | C | NT |
| 179 | A | NT |
| 180 | A | A |
| 181 | A | NT |
| 182 | B | NT |
| 183 | C | NT |
| 184 | A | NT |
| 185 | A | A |
| 186 | B | NT |
| 187 | B | NT |
| 188 | B | NT |
| 189 | C | NT |
| 190 | A | B |
| 191 | A | NT |
| 192 | NT | NT |
| 193 | NT | NT |
| 194 | A | NT |
| 195 | B | NT |
| 196 | B | NT |
| 197 | A | NT |
| 198 | A | NT |
| 199 | A | B |
| 200 | B | NT |
| 201 | A | NT |
| 202 | B | NT |
| 203 | A | NT |
| 204 | C | NT |
| 205 | C | NT |
| 206 | C | NT |
| 207 | A | NT |
| 208 | B | NT |
| 209 | B | NT |
| 210 | C | NT |
| 211 | B | NT |
| 212 | A | NT |
| 213 | B | NT |
| 214 | B | NT |
| 215 | C | NT |
| 216 | C | NT |
| 217 | B | NT |
| 218 | B | NT |
| 219 | B | NT |
| 180 | A | A |
| 181 | A | NT |
| 182 | B | NT |
| 183 | C | NT |
| 184 | A | NT |
| 185 | A | A |
| 186 | B | NT |
| 187 | B | NT |
| 188 | B | NT |
| 189 | C | NT |
| 190 | A | B |
| 191 | A | NT |
| 192 | NT | NT |
| 193 | NT | NT |
| 194 | A | NT |
| 195 | B | NT |
| 196 | B | NT |
| 197 | A | NT |
| 198 | A | NT |
| 199 | A | B |
| 200 | B | NT |
| 201 | A | NT |
| 202 | B | NT |
| 203 | A | NT |
| 204 | C | NT |
| 205 | C | NT |
| 206 | C | NT |
| 207 | A | NT |
| 208 | B | NT |
| 209 | B | NT |
| 210 | C | NT |
| 211 | B | NT |
| 212 | A | NT |
| 213 | B | NT |
| 214 | B | NT |
| 215 | C | NT |
| 216 | C | NT |
| 217 | B | NT |
| 218 | B | NT |
| 219 | B | NT |
| 220 | NT | NT |
| 221 | C | B |
| 222 | B | NT |
| 223 | B | NT |
| 224 | B | NT |
| 225 | B | NT |
| 226 | A | NT |
| 227 | B | NT |
| 228 | B | NT |
| 229 | A | NT |
| 230 | B | NT |
| 231 | C | NT |
| 232 | A | A |
| 233 | B | NT |
| 234 | A | NT |
| 235 | C | NT |
| 236 | B | NT |
| 237 | NT | NT |
| 238 | C | NT |
| 239 | C | NT |
| 240 | NT | NT |
| 241 | B | NT |
| 242 | B | NT |
| 243 | A | NT |
| 244 | C | NT |
| 245 | B | NT |
| 246 | C | B |
| 247 | C | NT |
| 248 | NT | NT |
| 249 | B | NT |
| 250 | C | C |
| 251 | A | A |
| 252 | C | NT |
| 253 | B | NT |
| 254 | NT | NT |
| 255 | B | NT |
| 256 | C | NT |
| 257 | C | NT |
| 258 | B | NT |
| 259 | B | NT |
| 260 | C | NT |
| 261 | A | A |
| 262 | B | NT |
| 263 | B | B |
| 264 | A | B |
| 265 | B | NT |
| 266 | B | NT |
| 267 | A | A |
| 268 | C | NT |
| 269 | NT | B |
| 270 | NT | NT |
| 271 | C | NT |
| 272 | B | NT |
| 273 | C | C |
| 274 | B | NT |
| 275 | B | NT |
| 276 | C | NT |
| 277 | B | B |
| 278 | B | B |
| 279 | C | C |
| 280 | NT | B |
| 281 | NT | B |
| 282 | NT | B |
| 283 | C | C |
| 284 | B | B |
| 285 | A | A |
| 286 | C | C |
| 287 | B | B |
| 289 | B | B |
| 290 | B | B |
| 291 | B | B |
| 292 | B | B |
| 293 | NT | B |
| 294 | NT | A |
| 295 | NT | C |
| 296 | NT | C |
| 297 | NT | A |
| 298 | NT | B |
| 299 | NT | C |
| 300 | NT | B |
| 301 | NT | C |
| 302 | NT | B |
| 303 | NT | B |
| 304 | NT | C |
| 305 | NT | C |
| 306 | NT | B |
| 307 | NT | C |
| 308 | NT | B |
| 309 | NT | C |
| 310 | NT | B |
| 311 | NT | C |
| 312 | NT | B |
| 313 | NT | B |
| 314 | NT | B |
| 315 | NT | C |
| 316 | NT | B |
| 317 | NT | C |
| 318 | NT | B |
| 319 | NT | C |
| 320 | NT | C |
| 321 | NT | B |
| 322 | NT | B |
| 323 | NT | B |
| 324 | NT | B |
| 325 | NT | B |
| 326 | NT | B |
| 327 | NT | B |
| 328 | NT | A |
| 329 | NT | B |
| 330 | NT | A |
| 331 | NT | B |
| 332 | NT | A |
| 333 | NT | C |
| 334 | NT | B |
| 335 | NT | B |
| 336 | NT | A |
| 337 | NT | B |
| 338 | NT | B |
| 339 | NT | C |
| 340 | NT | C |
| 341 | NT | C |
| 342 | NT | C |
| 343 | NT | B |
| 344 | NT | B |
| 345 | NT | C |
| 346 | NT | B |
| 347 | NT | B |
| 348 | NT | B |
| 349 | NT | B |
| 350 | NT | B |
| 351 | NT | A |
| 352 | NT | C |
| 353 | NT | A |
| 354 | NT | C |
| 355 | NT | B |
| 356 | NT | B |
| 357 | NT | C |
| 358 | NT | B |
| 359 | NT | A |
| 360 | NT | C |
| 361 | NT | B |
| 362 | NT | NT |
| 363 | NT | NT |
| 364 | NT | C |
| 365 | NT | B |
| 366 | NT | C |
| 367 | NT | C |
| 368 | NT | C |
| 369 | NT | B |
| 370 | NT | B |
| 371 | NT | A |
| 372 | NT | B |
| 373 | NT | C |
| 374 | NT | A |
| 375 | NT | B |
| 376 | NT | B |
| 377 | NT | B |
| 378 | NT | C |
| 379 | NT | C |
| 380 | NT | A |
| 381 | NT | C |
| 382 | NT | A |

1. A method of treating cancer in a selected patient, the method comprising administering to the patient a therapeutically effective amount of a compound of structural Formula (I): 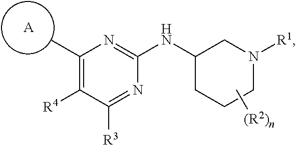
or a pharmaceutically acceptable salt thereof, wherein
ring A is a bicyclic 6,5-ring system selected from: 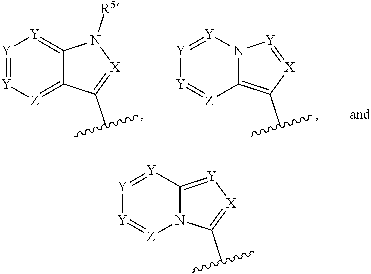
and comprises no more than four ring nitrogen atoms;
X is N or C(R⁶);
each Y is, independently, N or C(R⁷);
Z is N or C(R⁸);
R¹ is hydrogen, —C₁-C₆ alkyl, —O—(C₁-C₆-alkylene)-O—(C₁-C₄-alkyl), —(C₀-C₆ alkylene)-carbocyclyl, —C(O)—O—(C₁-C₆ alkylene), —(C₁-C₆ alkylene)-heterocyclyl, —(C₁-C₆ alkylene)-heteroaryl, —(C₁-C₆ alkylene)-N(R^{1′})₂, —(C₁-C₆ alkylene)-NR^{1′}—S(O)₂—(C₁-C₄ alkyl), —(C₁-C₆ alkylene)-NR^{1′}—SO₂—N(R^{1′})₂, —(C₁-C₆ alkylene)-S(O)₂—(C₁-C₄ alkyl), or —(C₁-C₆ alkylene)-S(O)₂—N(R^{1′})₂, wherein any carbocyclyl, heterocyclyl or heteroaryl portion of R¹ is optionally substituted, and wherein any alkyl or alkylene portion of R¹ is optionally substituted with one or more independently selected monovalent substituents;
each R^{1′} is, independently, hydrogen or optionally substituted C₁-C₆ alkyl, or
two R^{1′} are optionally taken together with the nitrogen atom to which they are bound to form a 4-6 membered, optionally substituted heterocyclyl or heteroaryl ring comprising up to 2 additional heteroatoms selected from N, O, and S, wherein:
each R², if present, is, independently, halo, —OH, —CN, —C₁-C₆ alkyl, —(C₀-C₆ alkylene)-carbocyclyl, —(C₀-C₆ alkylene)-heterocyclyl, —(C₀-C₆ alkylene)-heteroaryl, —(C₀-C₆ alkylene)-aryl, —(C₀-C₆ alkylene)-C(O)-heterocyclyl, —(C₀-C₆ alkylene)-C(O)-heteroaryl, —O—(C₁-C₆-alkyl), —O—(C₁-C₆-alkylene)-O—(C₁-C₄-alkyl), —O—(C₁-C₄-alkylene)-carbocyclyl, —O—(C₁-C₆-alkylene)-heterocyclyl, —O—(C₁-C₆-alkylene)-heteroaryl, —O—(C₁-C₆-alkylene)-aryl, —NH—C(O)—C₁-C₄ alkyl, or —C(O)—NH— (unsubstituted C₁-C₄ alkyl), or
R¹ and any R² are taken together with the atoms to which they are bound to form an optionally substituted heterocyclyl or heteroaryl ring fused, spirofused or bridged to the piperidine ring, or
two R² are taken together to form oxo, or taken together with the atom or atoms to which they are bound and any intervening ring atoms to form an optionally substituted aryl, carbocyclyl, heterocyclyl or heteroaryl ring fused, spirofused or bridged to the piperidine ring,
wherein any carbocyclyl, heterocyclyl, or heteroaryl portion of R², any ring formed by taking R¹ together with R², or any ring formed by taking two R² together is optionally substituted, and wherein any alkyl or alkylene portion of R² is optionally substituted with one or more independently selected monovalent substituents unless otherwise specified;
R³ is hydrogen, halo, —CN, optionally substituted —C₁-C₆ alkyl, or optionally substituted carbocyclyl;
R⁴ is halo, —CN, —C₁-C₆ alkyl, —C₂-C₆ alkenyl, C₂-C₆ alkynyl, —O—C₁-C₆ alkyl, —S—C₁-C₆ alkyl, or carbocyclyl, wherein any alkyl, alkenyl, alkynyl, or carbocyclyl portion of R⁴ is optionally substituted;
each R⁵ is, independently, halo, —OH, —C₁-C₆ alkyl, —CN, —(C₀-C₆ alkylene)-C(O)OH, —(C₀-C₆ alkylene)-C(O)—(C₁-C₄ alkyl), —(C₀-C₆ alkylene)-C(O)—N(R^{1′})₂, —(C₀-C₆ alkylene)-S(O)₂—(C₁-C₄ alkyl), —(C₀-C₆ alkylene)-S(O)₂—N(R^{1′})₂, —(C₀-C₆ alkylene)-P(O)—O—(C₁-C₄ alkyl)₂, —(C₀-C₆ alkylene)-P(O)—(C₁-C₄ alkyl)(O—C₁-C₄ alkyl), —(C₀-C₆ alkylene)-P(O)(C₁-C₄ alkyl)₂, —(C₀-C₆ alkylene)-carbocyclyl, —(C₀-C₆ alkylene)-heterocyclyl, —(C₀-C₆ alkylene)-heteroaryl, —(C₀-C₆ alkylene)-C(O)-heterocyclyl, —(C₀-C₆ alkylene)-C(O)-heteroaryl, —O—(C₁-C₆-alkyl), —O—(C₁-C₆-alkylene)-O—(C₁-C₄-alkyl), —O—(C₀-C₆-alkylene)-carbocyclyl, —O—(C₁-C₆-alkylene)-heterocyclyl, —O—(C₁-C₆-alkylene)-heteroaryl, phenyl, —(C₂-C₄ alkenylene)-phenyl, —S(O)—(C₁-C₄ alkyl), —S—(C₁-C₄ alkyl), —S(O)—OH, or —S(O)₂—OH, wherein any alkyl, alkylene, alkenylene, carbocyclyl, heterocyclyl, phenyl, and heteroaryl portion of R⁵ is optionally substituted; or
two vicinal R⁵ are taken together with the ring atoms to which they are bound to form an optionally substituted carbocyclyl or optionally substituted heterocyclyl, wherein each carbocyclyl or heterocyclyl is fused to ring A;
R^{5′} is hydrogen, —CN, —C₁-C₆ alkyl, —(C₀-C₆ alkylene)-S(O)₂—N(R^{1′})₂, —(C₀-C₆ alkylene)-carbocyclyl, —(C₀-C₆ alkylene)-C(O)—N(R^{1′})₂, —(C₀-C₆ alkylene)-aryl, —(C₀-C₆ alkylene)-heterocyclyl, —(C₀-C₆ alkylene)-heteroaryl, —(C₀-C₆ alkylene)-S(O)₂—(C₁-C₄ alkyl), —(C₁-C₆ alkylene)-O—(C₁-C₃ alkylene)-C(O)—N(R^{1′})₂, —(C₁-C₆ alkylene)-O—(C₁-C₄ alkylene)-P(O)(C₁-C₄ alkyl)₂, —(C₁-C₆ alkylene)-O—(C₁-C₄ alkylene)-P(O)(C₁-C₄ alkyl)-O—(C₁-C₄ alkyl), —(C₁-C₆ alkylene)-O—(C₁-C₄ alkylene)-P(O)—(O—C₁-C₄ alkyl)₂, —(C₁-C₆ alkylene)-O—(C₁-C₄ alkylene)-S(O)₂—(C₁-C₄ alkyl), —(C₁-C₆ alkylene)-O—(C₁-C₄ alkylene)-S(O)₂—N(R^{1′})₂, —(C₁-C₆ alkylene)-O—(C₁-C₄ alkyl), —(C₁-C₆ alkylene)-O-carbocyclyl, —(C₁-C₆ alkylene)-O-heteroaryl, —(C₁-C₆ alkylene)-O-heterocyclyl, —(C₁-C₆ alkylene)-P(O)(C₁-C₄ alkyl)₂, —(C₁-C₆ alkylene)-P(O)(C₁-C₄ alkyl)-O—(C₁-C₄ alkyl), —(C₁-C₆ alkylene)-P(O)—(O—C₁-C₄ alkyl)₂, —(C₁-C₆ alkylene)-C(O)—(C₁-C₄ alkyl), and —(C₁-C₆ alkylene)-C(O)OH, wherein any alkyl, alkylene, carbocyclyl, heterocyclyl and heteroaryl portion of R^{5′} is optionally substituted;
R^{5′} and any R⁵ are taken together with the ring atoms to which they are bound to form an optionally substituted heterocyclyl, wherein each heterocyclyl is fused to ring A;
R⁶ is hydrogen, —CN, —CH₃, —CH₂F, —CHF₂ or —CF₃;
each R⁷ is, independently, hydrogen or R⁵;
R⁸ is hydrogen or fluoro; and
n is 0, 1, 2, 3, or 4;
wherein the compound is other than one of the following compounds or a pharmaceutically acceptable salt thereof: 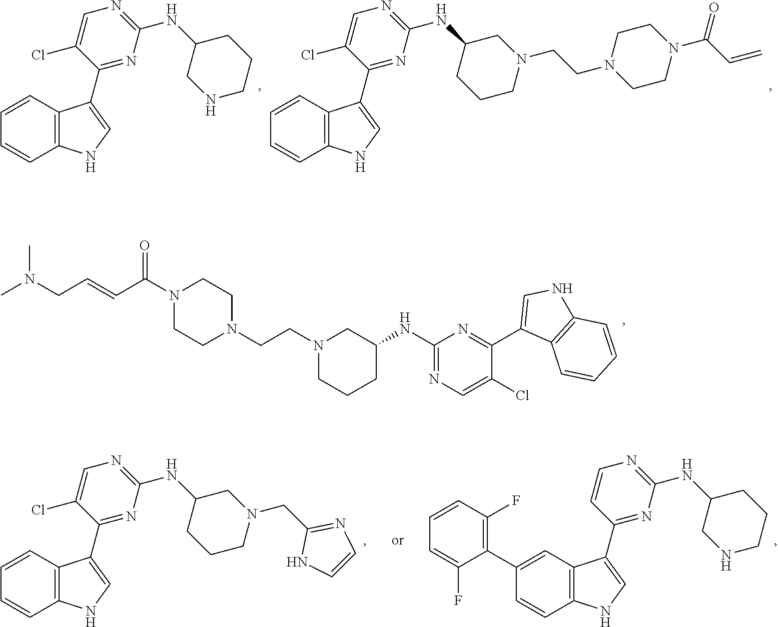
wherein the patient has been determined to have a cancer in which
(a) a gene selected from RB1, RBL1, RBL2, CDKN2A, CDKN2B, CDKN2C, CDKN2D, CDKN1A, CDKN1B CDKN1C, and FBWX7 is mutated, is genetically deleted, contains an epigenetic alteration, is translocated, is transcribed at a level equal to or below a pre-determined threshold, or encodes a protein that is translated at a level equal to or below a pre-determined threshold or has decreased activity relative to a reference standard;
(b) a gene selected from E2F1, E2F2, E2F3, E2F4, E2F5, E2F6, E2F7, E2F8, CDK1, CDK2, CDK4, CDK6, CCNA1, CCNB1, CCND1, CCND2, CCND3, CCNE1, and CCNE2 is mutated, is genetically gained or amplified, contains an epigenetic alteration, is translocated, transcribed at a level equal to or above a pre-determined threshold, or encodes a protein that is translated at a level equal to or above a pre-determined threshold or has increased activity relative to a reference standard; or
(c) the gene Bcl2-like 1 is mutated, contains an epigenetic alteration, is translocated, is transcribed at a level equal to or below a pre-determined threshold, or encodes a BCL-xL protein that is translated at a level equal to or below a pre-determined threshold or has decreased activity relative to a reference standard.

2. The method of claim 1, wherein ring A is:

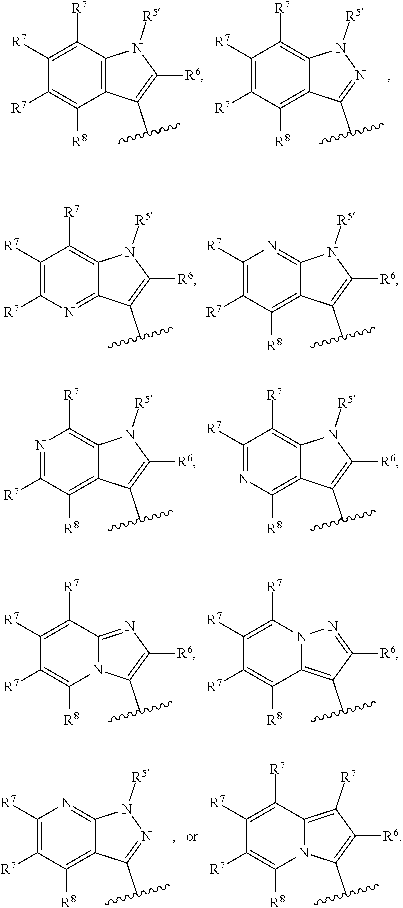

3. (canceled)

4. The method of claim 1, wherein R¹ is hydrogen, cyclopropyl, —CH₃, —CH₂CH₃, —CH₂CH₂OCH₃, —CH(CH₃)₂, or —CH₂CH(CH₃)₂, or wherein R¹ is taken together with one R² and the ring atoms to which each are bound to form a ring which, taken together with the ring to which R¹ and R² are bound, is

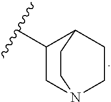 [SMILES: *C1CN2CCC1CC2]

5. (canceled)

6. The method of claim 1, wherein n is 0, 1, 2 or 3, and each R², if present, is, independently, fluoro, —CH₃, —CH₂CH₃, —OH, —CH(CH₃)₂, —C(O)NHCH₃, —NHC(O)CH₂CH₃, 3-methyl-1,2,4-oxadiazol-5-yl, 1,2,4-triazolo[4,3-a]pyridin-3-yl, 8-methylsulfonyl-1,2,4-triazolo[4,3-a]pyridin-3-yl, pyrrolidin-1-ylcarbonyl, 3-hydroxypyrrolidin-1-ylcarbonyl, or unsubstituted phenyl, or
two R² on different atoms are taken together with the atoms to which they are bound and any intervening ring atoms to form a ring which, taken together with the piperidine ring to which both R² are bound, is 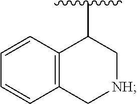 [SMILES: *C1CNCc2ccccc21]
or
two R² bound to the same ring atom are taken together to form oxo, or taken together with the atom to which they are bound to form a ring which, taken together with the piperidine ring to which both R² are bound, is

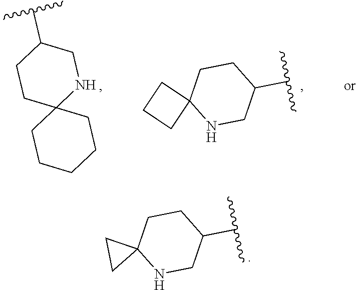 [SMILES: *C1CCC2(CCCCC2)NC1 ; *C1CCC2(CCC2)NC1 ; *C1CCC2(CC2)NC1]

7. The method of claim 1, wherein n is 0, 1, 2 or 3, and each R², if present, is, independently, fluoro, —CH₃, —CH₂CH₃, —OH, —CH(CH₃)₂, —C(O)NHCH₃, —NHC(O)CH₂CH₃, 3-methyl-1,2,4-oxadiazol-5-yl, 1,2,4-triazolo[4,3-a]pyridin-3-yl, 8-methylsulfonyl-1,2,4-triazolo[4,3-a]pyridin-3-yl, pyrrolidin-1-ylcarbonyl, 3-hydroxypyrrolidin-1-ylcarbonyl, or unsubstituted phenyl, or
two R² on different atoms are taken together with the atoms to which they are bound and any intervening ring atoms to form a ring which, taken together with the piperidine ring to which both R² are bound, is 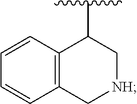 [SMILES: *C1CNCc2ccccc21]
or
two R² bound to the same ring atom are taken together to form oxo, or taken together with the atom to which they are bound to form a ring which, taken together with the piperidine ring to which both R² are bound, is:

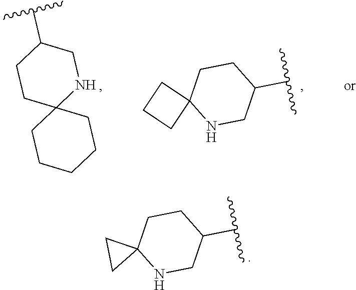 [SMILES: *C1CCC2(CCCCC2)NC1 ; *C1CCC2(CCC2)NC1 ; *C1CCC2(CC2)NC1]

8.-9. (canceled)

10. The method of claim 1, wherein R⁴ is chloro, fluoro, bromo, iodo, cyclopropyl, —CN, —CF₃, —CH₂CF₃, —CH₂CH₂F, —CH₃, —CH₂CH₃, —CH₂CH₂CH₃, —CH(CH₃)₂, —CH₂CH(CH₃)₂, —OCH₃, —CH(OH)CH₃, —CH═CH₂, —C(O)CH₃, —OCHF₂, S—CH₃, —S—CHF₂, —S—CF₃, or —C≡CH; and/or
each R⁷ is hydrogen, fluoro, chloro, bromo, —CN, —CH₃, —CH₂CH₂C(CH₃)₂₀H, —C(O)—CH₃, —C(O)OH, —C(O)—NH—CH₃, —P(═O)(OCH₂CH₃)₂, —P(═O)(OCH₂CH₃)CH₃, —S(O)₂CH₃, —P(O)—(CH₃)₂, —P(O)—(CH₂CH₃)₂, —S(O)₂N(CH₃)₂, —S(O)₂CH(CH₃)₂, —S(O)₂CH₂F, —S(O)₂CHF₂, —SCHF₂, —S(O)CHF₂, —S(O)OH, —S(O)₂₀H, —S(O)₂NHCH₃, —(CH₂)₄CH₃, —CH₂S(O)₂CH₃, —S(O)₂—CH₂CH₃, 1H-pyrazol-4-yl, 1-methylpyrazol-4-yl, 1,3-dimethyl-pyrazol-4-yl, 5-methyl-1H-pyrazol-4-yl, 1-methyl-2-oxoimidazolidin-3-yl, 4-methylimidazol-1-yl, morpholin-4-yl, pyridin-4-yl, pyridazin-4-yl, 4-hydroxycyclohexyl, 4-hydroxy-4-methylcyclohexyl, 5-methyl-1,2,4-triazol-3-yl, 5-methyl, 1,2,4-oxadiazol-3-yl, 1,3-dimethylpyridazin-4-yl, 1,5-dimethylpyridazin-4-yl, 3-methyl-1H-pyridazin-4-yl, 1-(2-methyl-2-hydroxypropyl)pyridazin-4-yl, imidazol-1-yl, 1-methyl-5-cyanopyrrol-3-yl, 5-cyano-1H-pyrrol-3-yl, and pyridazin-4-yl, 1H-pyrazol-3-yl, 1-difluoromethyl-pyrazol-3-yl, 1-difluoromethyl-pyrazol-4-yl, 1-methylpyrazol-3-yl, 3-methyl-1H-pyrazol-4-yl, 3-methyl-3-hydroxypyrrolidin-1-ylcarbonyl, 3-hydroxypyrrolidin-1-ylcarbonyl, 4-hydroxycyclohexyl, 4-hydroxycyclohex-1-enyl, 1,1-dioxothiomorpholin-4-yl, 4-cyano-TH-imidazol-1-yl, 2,3-dimethyl-1,2,4-triazol-5-yl, 1,5-dimethyl-pyrazol-4-yl, pyridin-3-yl, 1-(2-methyl-2-hydroxypropan-1-yl)pyrazol-4-yl, pyrrolidin-1-yl, pyrrolidin-1-ylcarbonyl, 1H-pyrazol-2-yl, 3-hydroxy-3-trifluoromethylpyrrolidin-1-ylcarbonyl, 3-methoxypyrrolidin-1-ylcarbonyl, 3-cyanopyrrolidin-1-ylcarbonyl, 4-hydroxy-4-methylpiperindin-1-ylcarbonyl, 3-oxopyrrolidin-1-ylcarbonyl, 3-(pyrrolidin-1-ylcarbonyl)phenyl, 3-phenoxyphenyl, thiazol-2-yl, pyrazin-2-yl, 2,4-dioxo-1H,3H-pyrimidin-5-yl, 3-methyl-3-hydroxypyrrolidin-1-ylsulfonyl, 5-flluoropyridin-3-yl, 2-hydroxpyridin-3-yl, 3,3-difluoro-4-hydroxy, 3,5-dimethyloxazol-4-yl, 3-fluorophenyl, 4-methylpyridin-3-1,2-hydroxymethylpyridin-3-yl, 6-hydroxymethylpyridin-2-yl, 5-hydroxymethylpyridin-3-yl, 1-methyl-6-oxopyridin-3-yl, 4-aminosulfonylphenyl, 3-aminosulfonylphenyl, 3-hydroxy-3-ethylpyrrolidin-1-ylcarbonyl, 3-cyano-4-hydroxyphenyl, benzo[d]thiazol-6-yl, 2H-indazol-6-yl, 1H-benzoimidazol-5-yl, 2-oxo-3-cyano-4-methylpyridin-5-yl, 2-aminobenzo[d]thiazol-2-yl, 3-aminocarbonylphenyl, 6-trifluoromethyl-1H-pyrrolo[3,2-c]pyridin-3-yl, 2-aminoquinazolin-8-yl, styryl, 1-methyl-TH-indazol-6-yl, 2,3-dihydrobenzo[b][1,4]dioxin-7-yl, 2-ethoxyphenyl, 3-(2-hydroxyethyl)phenyl, 3-(methylcarbonylaminomethyl)phenyl, 1-methyl-6-trifluoromethyl-1H-pyrrolo[3,2-c]pyridin-3-yl quinolin-4-yl, isoquinolin-5-yl, isoquinolin-7-yl, or 2-oxo-3,4-dihydroquinolin-7-yl; and/or
R^{5′} is hydrogen, C₁-C₄ alkyl, —(C₀-C₃ alkylene)-aryl, or —(C₁-C₃ alkylene)-O—(C₁-C₄ alkyl) and/or
R⁶ is hydrogen or methyl.

11.-13. (canceled)

14. The method of claim 1, wherein the compound is a compound of Formula (II): 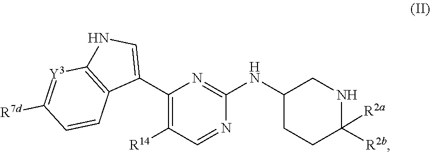
or a pharmaceutically acceptable salt thereof, wherein:
Y³ is N or C(R^{7e});
each of R^{2a} and R^{2b} is, independently, hydrogen or C₁-C₃ alkyl; or
R^{2a} and R^{2b} are taken together to form a cycloalkyl or a heterocycle spirofused to the piperidine ring, wherein said cycloalkyl or heterocycle is optionally substituted with one or more independently selected C₁-C₄ alkyl or C₁-C₄ haloalkyl;
R^{7d} is hydrogen, —C(O)—(C₁-C₄ alkyl), —CN, or heteroaryl optionally substituted with one or more independently selected C₁-C₄ alkyl or C₁-C₄ haloalkyl;
R^{7e}, if present, is hydrogen, halo, —S(O)₂—(C₁-C₄ alkyl), —P(O)(C₁-C₄ alkyl)₂, —C(O)NH—(C₁-C₄ alkyl), —C(O)N(C₁-C₄ alkyl)₂, —S(O)₂NH—(C₁-C₄ alkyl), —S(O)₂N—(C₁-C₄ alkyl)₂, or heteroaryl optionally substituted with one or more independently selected C₁-C₄ alkyl or C₁-C₄ haloalkyl; and
R¹⁴ is C₁-C₃ alkyl or C₁-C₃ haloalkyl.

15. The method of claim 14 or the pharmaceutically acceptable salt thereof, wherein
R^{2a} is hydrogen or —CH₃;
R^{2b} is hydrogen or —CH₃;
R^{7d} is hydrogen, —CN, pyrazin-2-yl, thiazol-2-yl, or 3,5-dimethylisoxazol-4-yl;
R^{7e}, if present, is hydrogen, fluoro, —C(O)NHCH₃, —P(O)(CH₃)₂, —S(O)₂CH₃, —S(O)₂N(CH₃)₂, 1,3-dimethylpyrazol-4-yl, or pyridazin-4-yl; and
R¹⁴ is —CH₂CH₃, or —CF₃.

16. Use A method of treating cancer in a selected patient, the method comprising administering to the patient a therapeutically effective amount of a compound of structural Formula (IV): 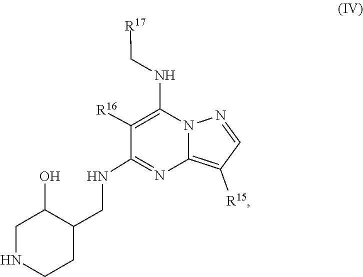
or a pharmaceutically acceptable salt thereof, wherein
R¹⁵ is hydrogen, C₁-C₆-alkyl or C₃-C₆-cycloalkyl, each optionally substituted by 1-3 halogens;
R¹⁶ is hydrogen, halogen, C₁-C₆-alkyl, or C₁-C₆-haloalkyl; and
R¹⁷ is phenyl optionally substituted with 1-3 substituents selected from the group consisting of halogen, —CN, C₁-C₆-alkyl, C₃-C₆-cycloalkyl, and C₁-C₆-haloalkyl,
wherein the patient has been determined to have a cancer in which
(a) a gene selected from RB1, RBL1, RBL2, CDKN2A, CDKN2B, CDKN2C, CDKN2D, CDKN1A, CDKN1B CDKN1C, and FBWX7 is mutated, is genetically deleted, contains an epigenetic alteration, is translocated, is transcribed at a level equal to or below a pre-determined threshold, or encodes a protein that is translated at a level equal to or below a pre-determined threshold or has decreased activity relative to a reference standard;
(b) a gene selected from E2F1, E2F2, E2F3, E2F4, E2F5, E2F6, E2F7, E2F8, CDK1, CDK2, CDK4, CDK6, CCNA1, CCNB1, CCND1, CCND2, CCND3, CCNE1, and CCNE2 is mutated, is genetically gained or amplified, contains an epigenetic alteration, is translocated, transcribed at a level equal to or above a pre-determined threshold, or encodes a protein that is translated at a level equal to or above a pre-determined threshold or has increased activity relative to a reference standard; or
(c) the gene Bcl2-like 1 is mutated, contains an epigenetic alteration, is translocated, is transcribed at a level equal to or below a pre-determined threshold, or encodes a BCL-xL protein that is translated at a level equal to or below a pre-determined threshold or has decreased activity relative to a reference standard.

17.-20. (canceled)

21. A method of treating cancer in a selected patient, the method comprising administering to the patient a therapeutically effective amount of the compound 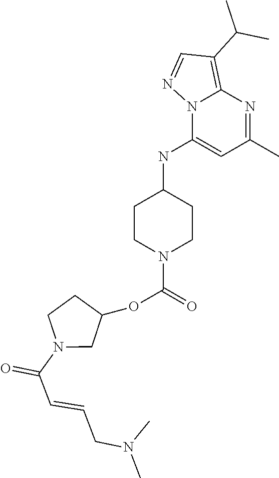 [SMILES: Cc1cc(NC2CCN(C(=O)OC3CCN(C(=O)/C=C/CN(C)C)C3)CC2)n2ncc(C(C)C)c2n1]
or a pharmaceutically acceptable salt thereof, wherein the patient has been determined to have a cancer in which
(a) a gene selected from RB1, RBL1, RBL2, CDKN2A, CDKN2B, CDKN2C, CDKN2D, CDKN1A, CDKN1B CDKN1C, and FBWX7 is mutated, is genetically deleted, contains an epigenetic alteration, is translocated, is transcribed at a level equal to or below a pre-determined threshold, or encodes a protein that is translated at a level equal to or below a pre-determined threshold or has decreased activity relative to a reference standard;
(b) a gene selected from E2F1, E2F2, E2F3, E2F4, E2F5, E2F6, E2F7, E2F8, CDK1, CDK2, CDK4, CDK6, CCNA1, CCNB1, CCND1, CCND2, CCND3, CCNE1, and CCNE2 is mutated, is genetically gained or amplified, contains an epigenetic alteration, is translocated, transcribed at a level equal to or above a pre-determined threshold, or encodes a protein that is translated at a level equal to or above a pre-determined threshold or has increased activity relative to a reference standard; or
(c) the gene Bcl2-like 1 is mutated, contains an epigenetic alteration, is translocated, is transcribed at a level equal to or below a pre-determined threshold, or encodes a BCL-xL protein that is translated at a level equal to or below a pre-determined threshold or has decreased activity relative to a reference standard.

22. A method of treating cancer in a selected patient, the method comprising administering to the patient a therapeutically effective amount of the compound 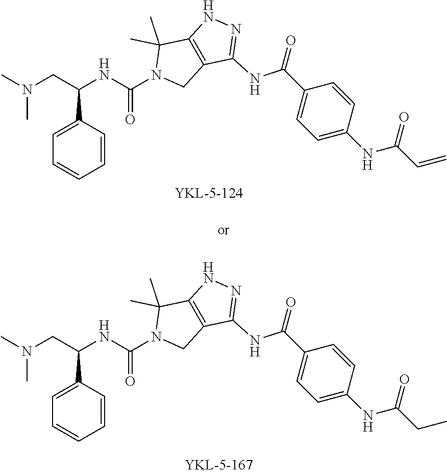
or a pharmaceutically acceptable salt of YKL-5-124 or YKL-5-167, wherein the patient has been determined to have a cancer in which
(a) a gene selected from RB1, RBL1, RBL2, CDKN2A, CDKN2B, CDKN2C, CDKN2D, CDKN1A, CDKN1B CDKN1C, and FBWX7 is mutated, is genetically deleted, contains an epigenetic alteration, is translocated, is transcribed at a level equal to or below a pre-determined threshold, or encodes a protein that is translated at a level equal to or below a pre-determined threshold or has decreased activity relative to a reference standard;
(b) a gene selected from E2F1, E2F2, E2F3, E2F4, E2F5, E2F6, E2F7, E2F8, CDK1, CDK2, CDK4, CDK6, CCNA1, CCNB1, CCND1, CCND2, CCND3, CCNE1, and CCNE2 is mutated, is genetically gained or amplified, contains an epigenetic alteration, is translocated, transcribed at a level equal to or above a pre-determined threshold, or encodes a protein that is translated at a level equal to or above a pre-determined threshold or has increased activity relative to a reference standard; or
(c) the gene Bcl2-like 1 is mutated, contains an epigenetic alteration, is translocated, is transcribed at a level equal to or below a pre-determined threshold, or encodes a BCL-xL protein that is translated at a level equal to or below a pre-determined threshold or has decreased activity relative to a reference standard.

23. The method of claim 1, wherein the cancer is a blood cancer, a breast cancer, Ewing's sarcoma, fallopian tube cancer, a GI tract cancer, a glioma, a lung cancer, melanoma, an osteosarcoma, an ovarian cancer, a pancreatic cancer, a primary peritoneal cancer, prostate cancer, retinoblastoma, or a squamous cell cancer of the head or neck.

24. (canceled)

25. The method of claim 23, wherein the patient has undergone, is presently undergoing, or is prescribed to undergo treatment with a Bcl-2 inhibitor.

26. The method of claim 25, wherein the Bcl-2 inhibitor is venetoclax and/or wherein the patient has a breast cancer; a blood cancer; an ovarian cancer; or a lung cancer.

27. (canceled)

28. The method of claim 23, wherein the patient has undergone, is presently undergoing, or is prescribed to undergo treatment with a selective estrogen receptor modulator (SERM); a selective estrogen receptor degrader (SERD); a PARP inhibitor; or a platinum-based therapeutic agent.

29. The method of claim 28, wherein the patient who has undergone, is presently undergoing, or is prescribed to undergo treatment: with a SERM or SERD has an HR+ breast cancer; with a PARP inhibitor has breast cancer, fallopian tube cancer, glioma, ovarian cancer, or primary peritoneal cancer; or with a platinum-based therapeutic agent has an ovarian cancer.

30. The method of claim 23, wherein the patient has undergone, is presently undergoing, or is prescribed to undergo treatment with a BET inhibitor with a CDK4/6 inhibitor; with a FLT3 inhibitor; or with a MEK inhibitor.

31. The method of claim 30, wherein the patient who has undergone, is presently undergoing, or is prescribed to undergo treatment, with a CDK4/6 inhibitor has a breast cancer, a pancreatic cancer, or a squamous cell cancer of the head or neck; with a FLT3 inhibitor has a blood cancer; with a BET inhibitor has a breast cancer, a blood cancer, Ewing's sarcoma, or an osteosarcoma.

32. The method of claim 1, wherein the patient has undergone, is presently undergoing, or is prescribed to undergo treatment with a second anti-cancer agent.

33. The method of claim 32, wherein the second anti-cancer agent is a Bcl-2 inhibitor; a CDK9 inhibitor; a hormone receptor degradation agent; a Flt3 (FMS-like tyrosine kinase 3) inhibitor; a PARP inhibitor; a BET inhibitor; a platinum-based therapeutic agent; a CDK4/6 inhibitor; a MEK inhibitor; a phosphoinositide 3-kinase (PI3 kinase) inhibitor; or capecitabine.

34. (canceled)
